(19) United States
(12) Patent Application Publication
(10) Pub. No.: US 2025/0327813 A1
(43) Pub. Date: Oct. 23, 2025

(54) CINNAMIC ACID DERIVATIVES AS MATRICES FOR MASS SPECTROMETRY

(71) Applicant: Vanderbilt University, Nashville, TN (US)

(72) Inventors: Jeffrey M. SPRAGGINS, Nashville, TN (US); Martin DUFRESNE, Nashville, TN (US)

(21) Appl. No.: 19/178,411

(22) Filed: Apr. 14, 2025

Related U.S. Application Data
(60) Provisional application No. 63/633,502, filed on Apr. 12, 2024.

(51) Int. Cl. G01N 33/68; G01N 1/38

(52) U.S. Cl. CPC G01N 33/6851; G01N 1/38

(57) ABSTRACT

The present invention provides matrices for use in mass spectrometry, particularly matrix-assisted laser desorption/ionization mass spectrometry techniques. Methods of use, including for imaging mass spectrometry, of the presently disclosed matrices are also provided.

### PRIORITY CLAIM

This application claims benefit of priority to U.S. Provisional Application Ser. No. 63/633,502, filed Apr. 12, 2024, the entire contents of which are hereby incorporated herein by reference.

### STATEMENT OF FEDERALLY SPONSORED RESEARCH

This invention was made with government support under grants U54DK134302 and from U54DK134302 from the National Institutes of Health. The government has certain rights in the invention.

### BACKGROUND

### Field

The present invention relates generally to the field of chemistry. More particularly, it relates to analytical methods that use a chemical matrix to enhance laser-based desorption and ionization of analyte molecules, such as matrix-assisted laser desorption/ionization mass spectrometry, as well as compounds and compositions for use with such analytical methods.

### Description of Related Art

Matrix-assisted laser desorption/ionization (MALDI) is the leading high spatial resolution (≤10 μm) imaging mass spectrometry (IMS) technology owing to its broad molecular coverage and ability to target and detect selected molecular classes through a wide variety of sample preparations. Recent advancements in instrumentation led to the acquisition of sub 10 μm MALDI IMS datasets being more common. While these have resulted in high-quality IMS images, smaller pixel sizes can lead to a decrease in sensitivity. Key factors such as the choice of matrix, the use of tissue washes, the matrix deposition method, and post-matrix application rehydration all significantly impact the ability to image analytes of interest (Zhou et al., 2021; Yang et al., 2018; Huang et al., 2020). Optimizing these processes has significant impacts on specificity, selectivity, and sensitivity, facilitating highly multiplexed molecular imaging at cellular resolution (≤10 μm pixel sizes) (Colley et al., 2024).

In a typical MALDI IMS workflow, molecular selectivity is influenced by the choice of matrix, the use of washing protocols, the method of applying said matrix, and post matrix application rehydration, with each of these having significant impact on specificity, selectivity, and sensitivity towards the molecular analytes of interests (Grove et al., 2011; Yang et al., 2011; Angel et al., 2012). The gains achieved by MALDI with its versatile sample preparation have yet to be surpassed by alternative ionization techniques, especially at high spatial resolution (≤10 μm) (Porta Siegel et al., 2018; Zavalin et al., 2012; Niehaus et al., 2019). Other techniques such as DESI, nano-DESI, LAESI, and IR-MALDESI, are making improvements in spatial resolution by minimizing the footprint of the probe on-tissue or using oversampling approaches (Yin et al., 2019; Unsihuay et al., 2021; Nazari & Muddiman, 2015). Although these techniques provide an ionization and sampling method for IMS platforms with minimal sample preparation, they often lack in sensitivity and image quality. MALDI IMS still provides the greatest spatial fidelity, allowing molecular signatures to be precisely linked to specific cell types and tissue structures in situ to support biomedical research applications (Yang et al., 2023; Ma et al., 2023; Djambazova et al., 2023).

There is a critical need to identify new dual-polarity chemical matrices for MALDI IMS to achieve high sensitivity for the targeted classes of analytes, vacuum stability, high extinction coefficient for typical MALDI lasers, functionality with both positive and negative ion modes (i.e., dual polarity), affordability, ease of use (e.g., easily applied, such as by sprayer or by sublimation), and low toxicity. Research in the development of MALDI matrices has been motivated by the desire to replace the commonly used 1,5-diaminonaphthalene (DAN), which is known to induce fragmentation and is highly toxic as a human cancerogenic and mutagenic (Zhou et al., 2021; Yang et al., 2018; Ma et al., 2023; Thomas et al., 2012; Lin et al., 2021). The first matrix to be discovered as a potential DAN replacement for dual polarity MALDI IMS of lipids was quercetin in 2013 by the Borcher group, which possessed many of the requirements but was only demonstrated to be efficient at low spatial resolution (100 μm), making it a poor candidate to replace DAN for high spatial resolution IMS applications (Wang et al., 2013). The second matrix to be found was norharmane in 2014 by the Vogel group and collaborators (Shirey et al., 2013; Scott et al., 2014). Norharmane has shown great potential in providing similar capabilities as DAN in term of sensitivity, molecular coverage, and vacuum stability but must be solubilized in a toxic solvent for use with automated robotic sprayers for application of the matrix to surfaces. Further, norharmane is expensive and has poor laser/matrix interaction requiring higher laser power and was significantly more expensive leading to its low adoption (Scott et al., 2016). A third matrix to demonstrate both positive and negative ionization capability similar to DAN was a modified version of anthranilic acid through a simple single methylation of its amino group by the Chou group (Huang et al., 2020). This was the first reported attempt at the rational design of a matrix for dual polarity lipid MALDI IMS but not the first in MALDI IMS in general (Huang et al., 2020; Yang et al., 2018). This new class of aminated benzoic acid matrices provided good molecular coverage, sensitivity, and had good laser/matrix interaction with mid-10³ extinction coefficient at 355 nm along with low toxicity while being relatively inexpensive. Unfortunately, this new matrix family is extremely volatile even for intermediate pressure MALDI source limiting their viability inside the mass spectrometer beyond 1-2 hours of data acquisition time. Thus, there remains a need for new chemical matrices with improved performance for MALDI experiments.

### SUMMARY

Briefly, the present disclosure provides compounds, compositions and methods for matrix-assisted laser desorption ionization (MALDI) mass spectrometry. In some embodiments, the matrices of the present invention may be deposited onto a tissue sample containing an analyte of interest. In some embodiments, the present methods and compounds enable analysis of such a tissue sample or portion of such a tissue sample, in either case optionally comprising an analyte of interest. Exemplary embodiments enable high spatial resolution imaging mass spectrometry. Exemplary embodiments allow highly controlled, repeatable deposition of matrix molecules onto solid substrates. In addition, exemplary embodiments have a high extinction coefficient at 355 nm. Exemplary embodiments further provide for vacuum stable MALDI matrices.

In one aspect, the present disclosure provides methods of detecting an analyte comprising:
a) obtaining a sample comprising an analyte;
b) obtaining a matrix compound of the formula:

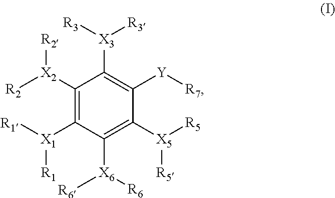

wherein:
X₁, X₂, X₃, X₅, and X₆ are each independently hydrogen, —C(O)—, or a proton-accepting atom, provided that if X₁ is hydrogen, R₁ and R_{1′} are absent; if X₃ is hydrogen, R₃ and R_{3′} are absent; and if X₅ is hydrogen, R₅ and R_{5′} are absent; and provided that at least one of X₁, X₂, X₃, X₅, and X₆ is not hydrogen;
R₁, R_{1′}, R₂, R_{2′}, R₃, R_{3′}, R₅, R_{5′}, R₆, and R_{6′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
Y is alkanediyl_{(C≤8)}, substituted alkanediyl_{(C≤8)}, alkenediyl_{(C≤8)}, substituted alkenediyl_{(C≤8)}, alkynediyl_{(C≤8)}, or substituted alkynediyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
   —C(O)R₈, wherein:
      R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
      alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof;
c) depositing the sample and the matrix compound on a surface to form an analyte-matrix co-crystal composition;
d) irradiating the analyte-matrix co-crystal composition with a laser;
e) detecting the analyte by mass spectrometry.

In some embodiments, the matrix compound is further defined as:

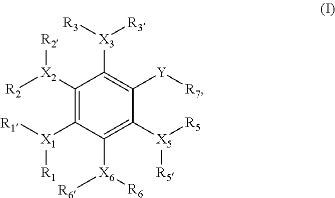

wherein:
X₁, X₂, X₃, X₅, and X₆ are each independently hydrogen, —C(O)—, or a proton-accepting atom, provided that if X₁ is hydrogen, R₁ and R_{1′} are absent; if X₃ is hydrogen, R₃ and R_{3′} are absent; and if X₅ is hydrogen, R₅ and R_{5′} are absent; and provided that at least one of X₁, X₂, X₃, X₅, and X₆ is not hydrogen;
R₁, R_{1′}, R₂, R_{2′}, R₃, R_{3′}, R₅, R_{5′}, R₆, and R_{6′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
Y is alkanediyl_{(C≤8)}, polar-substituted alkanediyl_{(C≤8)}, alkenediyl_{(C≤8)}, polar-substituted alkenediyl_{(C≤8)}, alkynediyl_{(C≤8)}, or polar-substituted alkynediyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
   —C(O)R₈, wherein:
      R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
         alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof;

In some embodiments, the matrix compound is further defined as:

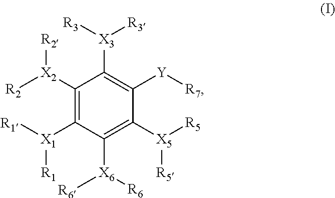

wherein:
X₁, X₂, X₃, X₅, and X₆ are each independently hydrogen, nitrogen, oxygen, sulfur, —C(O)—, or phosphorus provided that if X₁ is hydrogen, R₁ and R_{1′} are absent; if X₃ is hydrogen, R₃ and R_{3′} are absent; and if X₅ is hydrogen, R₅ and R_{5′} are absent; and provided that at least one of X₁, X₂, X₃, X₅, and X₆ is not hydrogen;
R₁, R_{1′}, R₂, R_{2′}, R₃, R_{3′}, R₅, R_{5′}, R₆, and R_{6′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
Y is alkanediyl_{(C≤8)}, polar-substituted alkanediyl_{(C≤8)}, alkenediyl_{(C≤8)}, polar-substituted alkenediyl_{(C≤8)}, alkynediyl_{(C≤8)}, or polar-substituted alkynediyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
   —C(O)R₈, wherein:
      R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
         alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof.

In some embodiments, the matrix compound is further defined as:

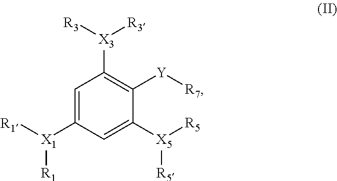

wherein:
X₁, X₃, and X₅ are each independently hydrogen, —C(O)—, or a proton-accepting atom, provided that if X₁ is hydrogen, R₁ and R_{1′} are absent; if X₃ is hydrogen, R₃ and R_{3′} are absent; and if X₅ is hydrogen, R₅ and R_{5′} are absent; and provided that at least one of X₁, X₃, and X₅ is not hydrogen;
R₁, R_{1′}, R₃, R_{3′}, R₅, and R_{5′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
Y is alkanediyl_{(C≤8)}, substituted alkanediyl_{(C≤8)}, alkenediyl_{(C≤8)}, substituted alkenediyl_{(C≤8)}, alkynediyl_{(C≤8)}, or substituted alkynediyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
   —C(O)R₈, wherein:
      R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
         alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof.

In some embodiments, the matrix compound is further defined as:

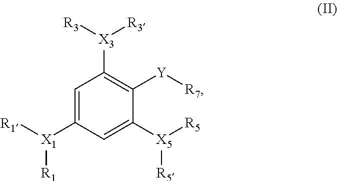

wherein:
X₁, X₃, and X₅ are each independently hydrogen, nitrogen, oxygen, sulfur, —C(O)—, or phosphorus provided that if X₁ is hydrogen, R₁ and R_{1′} are absent; if X₃ is hydrogen, R₃ and R_{3′} are absent; and if X₅ is hydrogen, R₅ and R_{5′} are absent; and provided that at least one of X₁, X₃, and X₅ is not hydrogen;
R₁, R_{1′}, R₃, R_{3′}, R₅, and R_{5′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
Y is alkanediyl_{(C≤8)}, polar-substituted alkanediyl_{(C≤8)}, alkenediyl_{(C≤8)}, polar-substituted alkenediyl_{(C≤8)}, alkynediyl_{(C≤8)}, or polar-substituted alkynediyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
   —C(O)R₈, wherein:
      R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
         alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof.

In some embodiments, the matrix compound is further defined as:

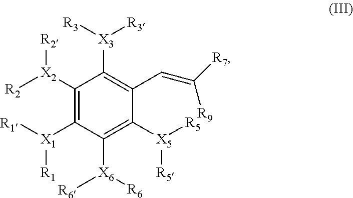

wherein:
X₁, X₂, X₃, X₅, and X₆ are each independently hydrogen, nitrogen, oxygen, sulfur, —C(O)—, or phosphorus provided that if X₁ is hydrogen, R₁ and R_{1′} are absent; if X₃ is hydrogen, R₃ and R_{3′} are absent; and if X₅ is hydrogen, R₅ and R_{5′} are absent; and provided that at least one of X₁, X₂, X₃, X₅, and X₆ is not hydrogen;
R₁, R_{1′}, R₂, R_{2′}, R₃, R_{3′}, R₅, R_{5′}, R₆, and R_{6′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
   —C(O)R₈, wherein:
      R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
         alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups; and
R₉ is hydrogen or an electron-withdrawing group.
or a salt thereof.

In some embodiments, the matrix compound is further defined as:

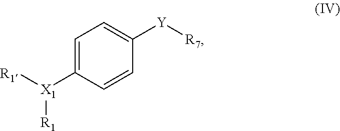

wherein:
   X₁ is nitrogen, oxygen, phosphorus, —C(O)—, or sulfur;
   R₁ and R_{1′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
   Y is alkanediyl_{(C≤8)}, substituted alkanediyl_{(C≤8)}, alkenediyl_{(C≤8)}, substituted alkenediyl_{(C≤8)}, alkynediyl_{(C≤8)}, or substituted alkynediyl_{(C≤8)};
   R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
      —C(O)R₈, wherein:
         R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
         alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof.

In some embodiments, the matrix compound is further defined as:

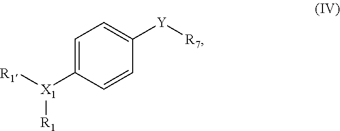

wherein:
   X₁ is nitrogen, oxygen, phosphorus, —C(O)—, or sulfur;
   R₁ and R_{1′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
   Y is alkanediyl_{(C≤8)}, polar-substituted alkanediyl_{(C≤8)}, alkenediyl_{(C≤8)}, polar-substituted alkenediyl_{(C≤8)}, alkynediyl_{(C≤8)}, or polar-substituted alkynediyl_{(C≤8)};
   R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
      —C(O)R₈, wherein:
         R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
         alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof.

In some embodiments, the matrix compound is further defined as:

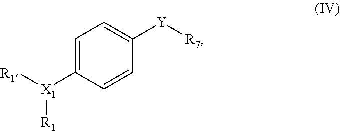

wherein:
   X₁ is nitrogen or —C(O)—;
   R₁ and R_{1′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
   Y is alkanediyl_{(C≤8)}, substituted alkanediyl_{(C≤8)}, alkenediyl_{(C≤8)}, substituted alkenediyl_{(C≤8)}, alkynediyl_{(C≤8)}, or substituted alkynediyl_{(C≤8)};
   R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
      —C(O)R₈, wherein:
         R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
         alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof.

In some embodiments, the matrix compound is further defined as:

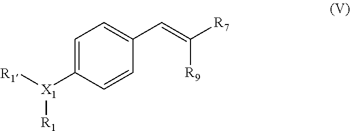

wherein:
   X₁ is nitrogen or —C(O)—;
   R₁ and R_{1′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
   R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
      —C(O)R₈, wherein:
         R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
         alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups; and
   R₉ is hydrogen or a polar group;
or a salt thereof.

In some embodiments, the matrix compound is further defined as:

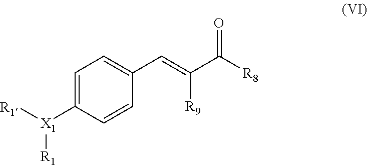

wherein:
   X₁ is nitrogen or —C(O)—;
   R₁ and R_{1′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
   R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
      alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups; and
   R₉ is hydrogen or a polar group;
or a salt thereof.

In some embodiments, X₁ is hydrogen. In other embodiments, X₁ is —C(O)—. In still other embodiments, X₁ is nitrogen, oxygen, sulfur, or phosphorus. In certain embodiments, X₁ is nitrogen. In some embodiments, X₂ is hydrogen. In other embodiments, X₂ is —C(O)—. In still other embodiments, X₂ is nitrogen, oxygen, sulfur, or phosphorus. In certain embodiments, X₂ is nitrogen. In some embodiments, X₃ is hydrogen. In other embodiments, X₃ is nitrogen, oxygen, sulfur, or phosphorus. In certain embodiments, X₃ is nitrogen. In some embodiments, X₅ is hydrogen. In other embodiments, X₅ is nitrogen, oxygen, sulfur, or phosphorus. In certain embodiments, X₅ is nitrogen. In some embodiments, X₆ is hydrogen. In other embodiments, X₆ is —C(O)—. In still other embodiments, X₆ is nitrogen, oxygen, sulfur, or phosphorus. In certain embodiments, X₆ is nitrogen. In some embodiments, X₁ is nitrogen, X₂ is hydrogen, X₃ is hydrogen, X₅ is hydrogen and X₆ is hydrogen.

In some embodiments, R₁ or R_{1′} are absent. In other embodiments, R₁ or R_{1′} is hydrogen. In some embodiments, R₁ and R_{1′} are both hydrogen. In other embodiments, R₁ or R_{1′} is alkyl_{(C≤8)} or substituted alkyl_{(C≤8)}. In certain embodiments, R₁ or R_{1′} is alkyl_{(C≤8)}, such as methyl. In some embodiments, R₁ and R_{1′} are both methyl. In other embodiments, R₁ or R_{1′} is substituted alkyl_{(C≤8)}. In certain embodiments, R₁ or R_{1′} is substituted propyl, such as 3-propanamine. In other embodiments, R₁ or R_{1′} is alkoxy_{(C≤8)}. In some embodiments, R₁ is alkoxy_{(C≤8)} and R_{1′} is absent. In other embodiments, R₁ is alkyl_{(C≤8)} and R_{1′} is hydrogen. In certain embodiments, R₁ is methyl and R_{1′} is hydrogen.

In some embodiments, R₂ or R_{2′} are absent. In some embodiments, R₂ or R_{2′} is hydrogen. In some embodiments, R₂ and R_{2′} are both hydrogen. In other embodiments, R₂ or R_{2′} is alkyl_{(C≤8)} or substituted alkyl_{(C≤8)}. In some embodiments, R₂ or R_{2′} is alkyl_{(C≤8)}, such as methyl. In some embodiments, R₂ and R_{2′} are both methyl. In other embodiments, R₂ or R_{2′} is substituted alkyl_{(C≤8)}. In some embodiments, R₂ or R_{2′} is substituted propyl, such as 3-propanamine. In other embodiments, R₂ or R_{2′} is alkoxy_{(C≤8)}. In some embodiments, R₂ is alkoxy_{(C≤8)} and R_{2′} is absent. In some embodiments, R₂ is alkyl_{(C≤8)} and R_{2′} is hydrogen. In some embodiments, R₂ is methyl and R_{2′} is hydrogen.

In some embodiments, at least one of R₂, R_{2′}, R₃, R_{3′}, R₅, R_{5′}, R₆, or R_{6′} are absent. In other embodiments, at least one of R₂, R_{2′}, R₃, R_{3′}, R₅, R_{5′}, R₆, or R_{6′} are hydrogen. In still other embodiments, at least one of R₂, R_{2′}, R₃, R_{3′}, R₅, R_{5′}, R₆, or R_{6′} are alkyl_{(C≤8)} or substituted alkyl_{(C≤8)}. In yet other embodiments, at least one of R₂, R_{2′}, R₃, R_{3′}, R₅, R_{5′}, R₆, or R_{6′} are alkoxy_{(C≤8)} or substituted alkoxy_{(C≤8)}. In some embodiments, at least one of R₂, R_{2′}, R₃, R_{3′}, R₅, R_{5′}, R₆, or R_{6′} are cycloalkyl_{(C≤8)} or substituted cycloalkyl_{(C≤8)}. In other embodiments, at least one of R₂, R_{2′}, R₃, R_{3′}, R₅, R_{5′}, R₆, or R_{6′} are alkenyl_{(C≤8)} or substituted alkenyl_{(C≤8)}. In some embodiments, at least one of R₂, R_{2′}, R₃, R_{3′}, R₅, R_{5′}, R₆, or R_{6′} are alkynyl_{(C≤8)} or substituted alkynyl_{(C≤8)}.

In some embodiments, R₃ and R_{3′} are absent. In other embodiments, R₃ or R_{3′} is hydrogen. In some embodiments, R₃ and R_{3′} are both hydrogen. In other embodiments, R₃ or R_{3′} is alkyl_{(C≤8)} or substituted alkyl_{(C≤8)}. In certain embodiments, R₃ or R_{3′} is alkyl_{(C≤8)}, such as methyl. In some embodiments, R₃ and R_{3′} are both methyl. In some embodiments, R₃ is alkyl_{(C≤8)} and R_{3′} is hydrogen. In some embodiments, R₃ is methyl and R_{3′} is hydrogen. In some embodiments, R₅ and R_{5′} are absent. In some embodiments, R₅ or R_{5′} is hydrogen. In some embodiments, R₅ and R_{5′} are both hydrogen. In other embodiments, R₅ or R_{5′} is alkyl_{(C≤8)} or substituted alkyl_{(C≤8)}. In certain embodiments, R₅ or R_{5′} is alkyl_{(C≤8)}, such as methyl. In some embodiments, R₅ and R_{5′} are both methyl. In some embodiments, R₅ is alkyl_{(C≤8)} and R_{5′} is hydrogen. In some embodiments, R₅ is methyl and R_{5′} is hydrogen.

In some embodiments, Y is alkanediyl_{(C≤8)} or substituted alkanediyl_{(C≤8)}. In some embodiments, Y is alkenediyl_{(C≤8)} or substituted alkenediyl_{(C≤8)}. In certain embodiments, Y is alkenediyl_{(C≤8)}, such as ethenediyl. In some embodiments, Y is alkynediyl_{(C≤8)} or substituted alkynediyl_{(C≤8)}. In some embodiments, R₇ is cyano, alkylsulfonyl_{(C≤8)}, substituted alkylsulfonyl_{(C≤8)}, cycloalkylsulfonyl_{(C≤8)}, or substituted cycloalkylsulfonyl_{(C≤8)}. In other embodiments, R₇ is aryl_{(C≤12)} or substituted aryl_{(C≤12)}. In some embodiments, R₇ is substituted aryl_{(C≤12)}. In other embodiments, R₇ is —C(O)R₈, wherein:
R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups.

In some embodiments, R₇ is —C(O)R₈, wherein R₈ is hydrogen. In other embodiments, R₇ is —C(O)R₈, wherein R₈ is hydroxy. In still other embodiments, R₇ is —C(O)R₈, wherein R₈ is alkoxy_{(C≤8)} or substituted alkoxy_{(C≤8)}. In certain embodiments, R₇ is —C(O)R₈, wherein R₈ is alkoxy_{(C≤8)}, such as methoxy.

In some embodiments, the sample is a tissue sample, such as a human tissue sample. In some embodiments, the depositing of the sample and the matrix compound on a surface comprises:
(i) placing the sample on a surface;
(ii) solubilizing the matrix compound in a solvent to form a chemical matrix solution; and
(iii) spraying the chemical matrix solution on the sample.

In some embodiments, the solvent is an organic solvent, such as acetone, acetonitrile, dimethylformamide, tetrahydrofuran, methanol, ethanol, or a combination thereof. In some embodiments, the solvent comprises water. In some embodiments, the depositing of the sample and the matrix compound comprises sublimation at a first temperature and a first pressure for a first time period. In some embodiments, the first temperature is between about 100° C. and about 250° C. In certain embodiments, the first temperature is between about 150° C. and about 200° C. In certain embodiments, the first temperature is about 180° C. In some embodiments, the first pressure is between about 25 mbar and about 500 mbar. In certain embodiments, the first pressure is between about 100 mbar and about 200 mbar. In certain embodiments, the first pressure is about 110 mbar.

In some embodiments, the depositing of the sample and the matrix compound on a surface comprises placing the sample on a surface and spotting the matrix compound on the sample. In some embodiments, the method further comprises heating the analyte-matrix co-crystal composition to a second temperature for a second period of time before irradiation of the analyte-matrix co-crystal composition. In certain embodiments, the second temperature is between about 50° C. and about 150° C. In certain embodiments, the second temperature is between 75° C. and 125° C. In certain embodiments, the second temperature is about 100° C. In some embodiments, the second period of time is between about 5 seconds and about 60 seconds. In certain embodiments, the second period of time is between about 10 seconds and about 45 seconds. In certain embodiments, the second period of time is about 15 seconds. In other embodiments, the second period of time is about 30 seconds.

In some embodiments, the surface density of the analyte-matrix co-crystal is from about 0.1 μg/mm² to about 1 μg/mm². In certain embodiments, the surface density of the analyte-matrix co-crystal is from about 0.2 μg/mm² to about 0.75 μg/mm². In certain embodiments, the surface density of the analyte-matrix co-crystal is about 0.25 μg/mm². In some embodiments, the average crystal size of the analyte-matrix co-crystal is less than 5000 nm. In certain embodiments, the average crystal size of the analyte-matrix co-crystal is between about 100 nm and about 600 nm. In certain embodiments, the average crystal size of the analyte-matrix co-crystal is about 250 nm. In some embodiments, the wavelength of the laser is 355 nm. In some embodiments, the mass spectrometry is MALDI. In some embodiments, the analyte is a protein or a peptide. In some embodiments, the analyte is a molecular probe or mass-tag. In some embodiments, the analyte is a metabolite. In some embodiments, the analyte is a lipid. In some embodiments, the analyte is a phospholipid. In some embodiments, the analyte is a ganglioside.

In some aspects, the matrix compound is further defined as:

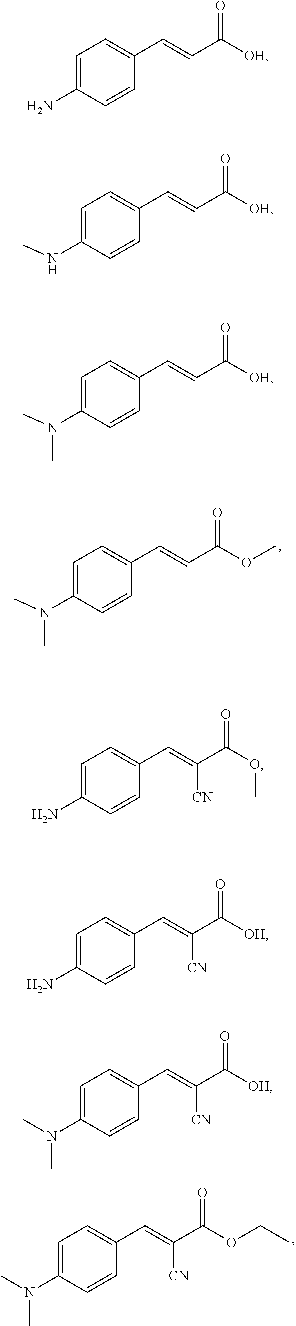

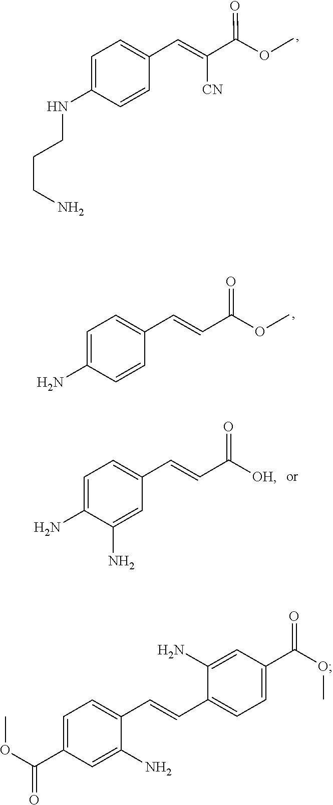 [SMILES: COC(=O)/C(C#N)=C/c1ccc(NCCCN)cc1 ; COC(=O)/C=C/c1ccc(N)cc1 ; Nc1ccc(/C=C/C(=O)O)cc1N ; COC(=O)c1ccc(/C=C/c2ccc(C(=O)OC)cc2N)c(N)c1]

or a salt thereof.

In one aspect, the present disclosure provides a matrix for use in matrix-assisted laser desorption/ionization, comprising a matrix compound having the formula:

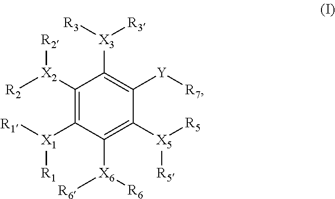

wherein:
X₁, X₂, X₃, X₅, and X₆ are each independently hydrogen, —C(O)—, or a proton-accepting atom, provided that if X₁ is hydrogen, R₁ and R_{1′} are absent; if X₃ is hydrogen, R₃ and R_{3′} are absent; and if X₅ is hydrogen, R₅ and R_{5′} are absent; and provided that at least one of X₁, X₂, X₃, X₅, and X₆ is not hydrogen;
R₁, R_{1′}, R₂, R_{2′}, R₃, R_{3′}, R₅, R_{5′}, R₆, and R_{6′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
Y is alkanediyl_{(C≤8)}, substituted alkanediyl_{(C≤8)}, alkenediyl_{(C≤8)}, substituted alkenediyl_{(C≤8)}, alkynediyl_{(C≤8)}, or substituted alkynediyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
   —C(O)R₈, wherein:
      R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
         alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof.

In some embodiments, the matrix compound is further defined as:

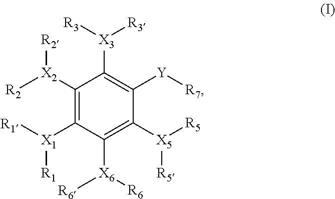

wherein:
X₁, X₂, X₃, X₅, and X₆ are each independently hydrogen, —C(O)—, or a proton-accepting atom, provided that if X₁ is hydrogen, R₁ and R_{1′} are absent; if X₃ is hydrogen, R₃ and R_{3′} are absent; and if X₅ is hydrogen, R₅ and R_{5′} are absent; and provided that at least one of X₁, X₂, X₃, X₅, and X₆ is not hydrogen;
R₁, R_{1′}, R₂, R_{2′}, R₃, R_{3′}, R₅, R_{5′}, R₆, and R_{6′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
Y is alkanediyl_{(C≤8)}, polar-substituted alkanediyl_{(C≤8)}, alkenediyl_{(C≤8)}, polar-substituted alkenediyl_{(C≤8)}, alkynediyl_{(C≤8)}, or polar-substituted alkynediyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
   —C(O)R₈, wherein:
      R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
         alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof;
In some embodiments, the matrix compound is further defined as:

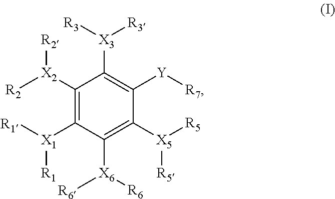

wherein:
X₁, X₂, X₃, X₅, and X₆ are each independently hydrogen, nitrogen, oxygen, sulfur, —C(O)—, or phosphorus provided that if X₁ is hydrogen, R₁ and R_{1′} are absent; if X₃ is hydrogen, R₃ and R_{3′} are absent; and if X₅ is hydrogen, R₅ and R_{5′} are absent; and provided that at least one of X₁, X₂, X₃, X₅, and X₆ is not hydrogen;
R₁, R_{1′}, R₂, R_{2′}, R₃, R_{3′}, R₅, R_{5′}, R₆, and R_{6′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
Y is alkanediyl_{(C≤8)}, polar-substituted alkanediyl_{(C≤8)}, alkenediyl_{(C≤8)}, polar-substituted alkenediyl_{(C≤8)}, alkynediyl_{(C≤8)}, or polar-substituted alkynediyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
   —C(O)R₈, wherein:
      R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
         alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof.

In some embodiments, the matrix compound is further defined as:

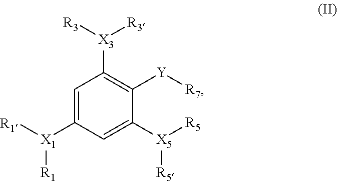

wherein:
X₁, X₃, and X₅ are each independently hydrogen, —C(O)—, or a proton-accepting atom, provided that if X₁ is hydrogen, R₁ and R_{1′} are absent; if X₃ is hydrogen, R₃ and R_{3′} are absent; and if X₅ is hydrogen, R₅ and R_{5′} are absent; and provided that at least one of X₁, X₃, and X₅ is not hydrogen;
R₁, R_{1′}, R₃, R_{3′}, R₅, and R_{5′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
Y is alkanediyl_{(C≤8)}, substituted alkanediyl_{(C≤8)}, alkenediyl_{(C≤8)}, substituted alkenediyl_{(C≤8)}, alkynediyl_{(C≤8)}, or substituted alkynediyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
   —C(O)R₈, wherein:
      R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
         alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof.

In some embodiments, the matrix compound is further defined as:

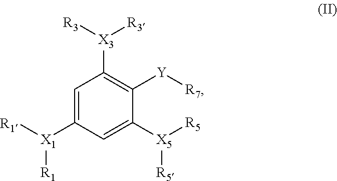

wherein:
X₁, X₃, and X₅ are each independently hydrogen, nitrogen, oxygen, sulfur, —C(O)—, or phosphorus provided that if X₁ is hydrogen, R₁ and R_{1′} are absent; if X₃ is hydrogen, R₃ and R_{3′} are absent; and if X₅ is hydrogen, R₅ and R_{5′} are absent; and provided that at least one of X₁, X₃, and X₅ is not hydrogen;
R₁, R_{1′}, R₃, R_{3′}, R₅, and R_{5′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
Y is alkanediyl_{(C≤8)}, polar-substituted alkanediyl_{(C≤8)}, alkenediyl_{(C≤8)}, polar-substituted alkenediyl_{(C≤8)}, alkynediyl_{(C≤8)}, or polar-substituted alkynediyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
   —C(O)R₈, wherein:
      R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
         alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof.

In some embodiments, the matrix compound is further defined as:

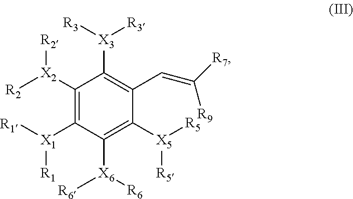

wherein:
X₁, X₂, X₃, X₅, and X₆ are each independently hydrogen, nitrogen, oxygen, sulfur, —C(O)—, or phosphorus provided that if X₁ is hydrogen, R₁ and R_{1′} are absent; if X₃ is hydrogen, R₃ and R_{3′} are absent; and if X₅ is hydrogen, R₅ and R_{5′} are absent; and provided that at least one of X₁, X₂, X₃, X₅, and X₆ is not hydrogen;
R₁, R_{1′}, R₂, R_{2′}, R₃, R_{3′}, R₅, R_{5′}, R₆, and R_{6′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
   —C(O)R₈, wherein:
      R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
         alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups; and
R₉ is hydrogen or an electron-withdrawing group.
or a salt thereof.

In some embodiments, the matrix compound is further defined as:

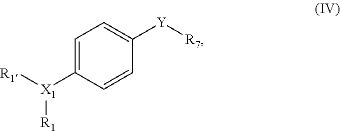

wherein:
   X₁ is nitrogen, oxygen, phosphorus, —C(O)—, or sulfur;
   R₁ and R_{1′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
   Y is alkanediyl_{(C≤8)}, substituted alkanediyl_{(C≤8)}, alkenediyl_{(C≤8)}, psubstituted alkenediyl_{(C≤8)}, alkynediyl_{(C≤8)}, or substituted alkynediyl_{(C≤8)};
   R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
      —C(O)R₈, wherein:
         R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
         alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof.

In some embodiments, the matrix compound is further defined as:

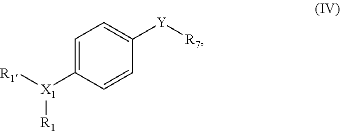

wherein:
   X₁ is nitrogen, oxygen, phosphorus, —C(O)—, or sulfur;
   R₁ and R_{1′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
   Y is alkanediyl_{(C≤8)}, polar-substituted alkanediyl_{(C≤8)}, alkenediyl_{(C≤8)}, polar-substituted alkenediyl_{(C≤8)}, alkynediyl_{(C≤8)}, or polar-substituted alkynediyl_{(C≤8)};
   R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
      —C(O)R₈, wherein:
         R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
         alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof.

In some embodiments, the matrix compound is further defined as:

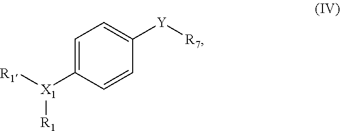

wherein:
   X₁ is nitrogen or —C(O)—;
   R₁ and R_{1′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
   Y is alkanediyl_{(C≤8)}, substituted alkanediyl_{(C≤8)}, alkenediyl_{(C≤8)}, substituted alkenediyl_{(C≤8)}, alkynediyl_{(C≤8)}, or substituted alkynediyl_{(C≤8)};
   R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
      —C(O)R₈, wherein:
         R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
         alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof.

In some embodiments, the matrix compound is further defined as:

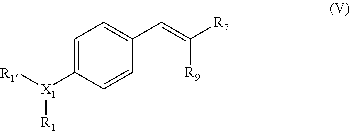

wherein:
   X₁ is nitrogen or —C(O)—;
   R₁ and R_{1′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
   R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
      —C(O)R₈, wherein:
         R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
         alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups; and
   R₉ is hydrogen or a polar group;
or a salt thereof.

In some embodiments, the matrix compound is further defined as:

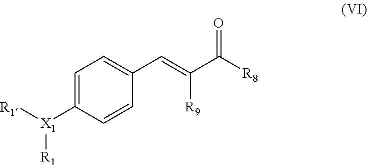

wherein:
   X₁ is nitrogen or —C(O)—;
   R₁ and R_{1′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
   R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
      alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups; and
   R₉ is hydrogen or a polar group;
or a salt thereof.

In some embodiments, X₁ is hydrogen. In other embodiments, X₁ is —C(O)—. In still other embodiments, X₁ is nitrogen, oxygen, sulfur, or phosphorus. In certain embodiments, X₁ is nitrogen. In some embodiments, X₂ is hydrogen. In other embodiments, X₂ is —C(O)—. In still other embodiments, X₂ is nitrogen, oxygen, sulfur, or phosphorus. In certain embodiments, X₂ is nitrogen. In some embodiments, X₃ is hydrogen. In other embodiments, X₃ is nitrogen, oxygen, sulfur, or phosphorus. In certain embodiments, X₃ is nitrogen. In some embodiments, X₅ is hydrogen. In other embodiments, X₅ is nitrogen, oxygen, sulfur, or phosphorus. In certain embodiments, X₅ is nitrogen. In some embodiments, X₆ is hydrogen. In other embodiments, X₆ is —C(O)—. In still other embodiments, X₆ is nitrogen, oxygen, sulfur, or phosphorus. In certain embodiments, X₆ is nitrogen. In some embodiments, X₁ is nitrogen, X₂ is hydrogen, X₃ is hydrogen, X₅ is hydrogen and X₆ is hydrogen.

In some embodiments, R₁ or R_{1′} are absent. In other embodiments, R₁ or R_{1′} is hydrogen. In some embodiments, R₁ and R_{1′} are both hydrogen. In other embodiments, R₁ or R_{1′} is alkyl_{(C≤8)} or substituted alkyl_{(C≤8)}. In certain embodiments, R₁ or R_{1′} is alkyl_{(C≤8)}, such as methyl. In some embodiments, R₁ and R_{1′} are both methyl. In other embodiments, R₁ or R_{1′} is substituted alkyl_{(C≤8)}. In certain embodiments, R₁ or R_{1′} is substituted propyl, such as 3-propanamine. In other embodiments, R₁ or R_{1′} is alkoxy_{(C≤8)}. In some embodiments, R₁ is alkoxy_{(C≤8)} and R_{1′} is absent. In other embodiments, R₁ is alkyl_{(C≤8)} and R_{1′} is hydrogen. In certain embodiments, R₁ is methyl and R_{1′} is hydrogen.

In some embodiments, R₂ or R_{2′} are absent. In some embodiments, R₂ or R_{2′} is hydrogen. In some embodiments, R₂ and R_{2′} are both hydrogen. In other embodiments, R₂ or R_{2′} is alkyl_{(C≤8)} or substituted alkyl_{(C≤8)}. In some embodiments, R₂ or R_{2′} is alkyl_{(C≤8)}, such as methyl. In some embodiments, R₂ and R_{2′} are both methyl. In other embodiments, R₂ or R_{2′} is substituted alkyl_{(C≤8)}. In some embodiments, R₂ or R_{2′} is substituted propyl, such as 3-propanamine. In other embodiments, R₂ or R_{2′} is alkoxy_{(C≤8)}. In some embodiments, R₂ is alkoxy_{(C≤8)} and R_{2′} is absent. In some embodiments, R₂ is alkyl_{(C≤8)} and R_{2′} is hydrogen. In some embodiments, R₂ is methyl and R_{2′} is hydrogen.

In some embodiments, at least one of R₂, R_{2′}, R₃, R_{3′}, R₅, R_{5′}, R₆, or R_{6′} are absent. In other embodiments, at least one of R₂, R_{2′}, R₃, R_{3′}, R₅, R_{5′}, R₆, or R_{6′} are hydrogen. In still other embodiments, at least one of R₂, R_{2′}, R₃, R_{3′}, R₅, R_{5′}, R₆, or R_{6′} are alkyl_{(C≤8)} or substituted alkyl_{(C≤8)}. In yet other embodiments, at least one of R₂, R_{2′}, R₃, R_{3′}, R₅, R_{5′}, R₆, or R_{6′} are alkoxy_{(C≤8)} or substituted alkoxy_{(C≤8)}. In some embodiments, at least one of R₂, R_{2′}, R₃, R_{3′}, R₅, R_{5′}, R₆, or R_{6′} are cycloalkyl_{(C≤8)} or substituted cycloalkyl_{(C≤8)}. In other embodiments, at least one of R₂, R_{2′}, R₃, R_{3′}, R₅, R_{5′}, R₆, or R_{6′} are alkenyl_{(C≤8)} or substituted alkenyl_{(C≤8)}. In some embodiments, at least one of R₂, R_{2′}, R₃, R_{3′}, R₅, R_{5′}, R₆, or R_{6′} are alkynyl_{(C≤8)} or substituted alkynyl_{(C≤8)}.

In some embodiments, R₃ and R_{3′} are absent. In other embodiments, R₃ or R_{3′} is hydrogen. In some embodiments, R₃ and R_{3′} are both hydrogen. In other embodiments, R₃ or R_{3′} is alkyl_{(C≤8)} or substituted alkyl_{(C≤8)}. In certain embodiments, R₃ or R_{3′} is alkyl_{(C≤8)}, such as methyl. In some embodiments, R₃ and R_{3′} are both methyl. In some embodiments, R₃ is alkyl_{(C≤8)} and R_{3′} is hydrogen. In some embodiments, R₃ is methyl and R_{3′} is hydrogen. In some embodiments, R₅ and R_{5′} are absent. In some embodiments, R₅ or R_{5′} is hydrogen. In some embodiments, R₅ and R_{5′} are both hydrogen. In other embodiments, R₅ or R_{5′} is alkyl_{(C≤8)} or substituted alkyl_{(C≤8)}. In certain embodiments, R₅ or R_{5′} is alkyl_{(C≤8)}, such as methyl. In some embodiments, R₅ and R_{5′} are both methyl. In some embodiments, R₅ is alkyl_{(C≤8)} and R_{5′} is hydrogen. In some embodiments, R₅ is methyl and R_{5′} is hydrogen.

In some embodiments, Y is alkanediyl_{(C≤8)} or substituted alkanediyl_{(C≤8)}. In some embodiments, Y is alkenediyl_{(C≤8)} or substituted alkenediyl_{(C≤8)}. In certain embodiments, Y is alkenediyl_{(C≤8)}, such as ethenediyl. In some embodiments, Y is alkynediyl_{(C≤8)} or substituted alkynediyl_{(C≤8)}. In some embodiments, R₇ is cyano, alkylsulfonyl_{(C≤8)}, substituted alkylsulfonyl_{(C≤8)}, cycloalkylsulfonyl_{(C≤8)}, or substituted cycloalkylsulfonyl_{(C≤8)}. In other embodiments, R₇ is aryl_{(C≤12)} or substituted aryl_{(C≤12)}. In some embodiments, R₇ is substituted aryl_{(C≤12)}. In other embodiments, R₇ is —C(O)R₈, wherein:
R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups.

In some embodiments, R₇ is —C(O)R₈, wherein R₈ is hydrogen. In other embodiments, R₇ is —C(O)R₈, wherein R₇ is hydroxy. In still other embodiments, R₇ is —C(O)R₈, wherein R₈ is alkoxy_{(C≤8)} or substituted alkoxy_{(C≤8)}. In certain embodiments, R₇ is —C(O)R₈, wherein R₈ is alkoxy_{(C≤8)}, such as methoxy.

In some embodiments, the matrix has been deposited on a sample. In some embodiments, the matrix has been deposited on the sample by spraying. In some embodiments, the matrix has been deposited on the sample by sublimation. In some embodiments, the matrix has been deposited on the sample by spotting. In some embodiments, the matrix compound is further defined as:

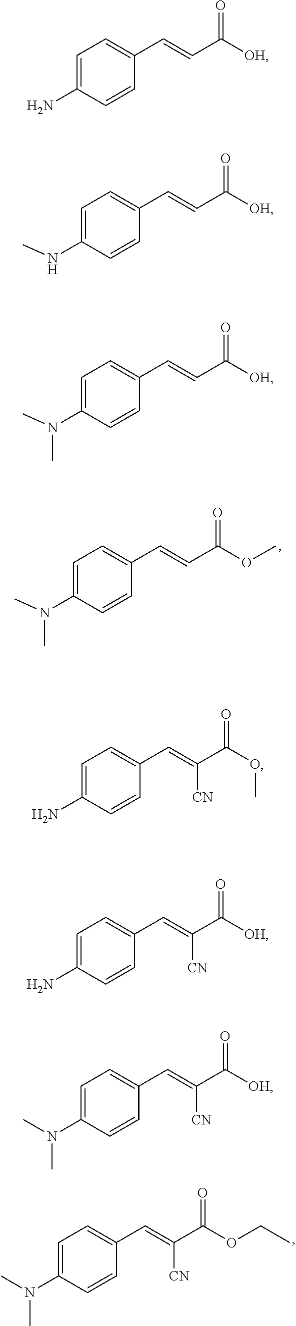

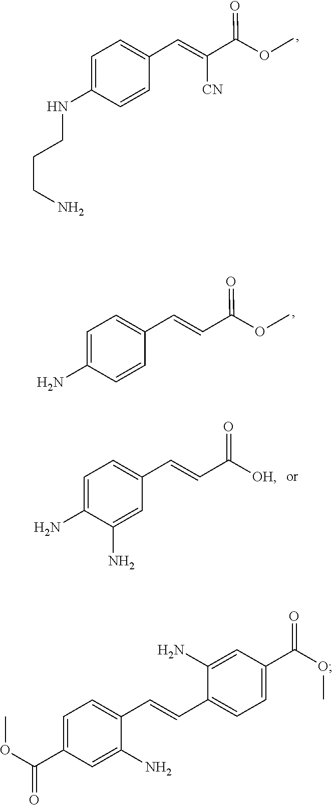 [SMILES: COC(=O)/C(C#N)=C/c1ccc(NCCCN)cc1 ; COC(=O)/C=C/c1ccc(N)cc1 ; Nc1ccc(/C=C/C(=O)O)cc1N ; COC(=O)c1ccc(/C=C/c2ccc(C(=O)OC)cc2N)c(N)c1]

or a salt thereof.

In some aspects, the present disclosure provides a compound of the formula:

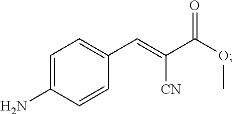

or a salt thereof.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIGS. 1A-1B shows scanning electron microscope (SEM) images of a thin tissue sample. FIG. 1A shows an SEM image of a thin tissue sample with corresponding software ablation crater measurement (table at right). The table provides ablation craters measurement for three separate SEM acquisitions, both in vertical (odd numbers) and horizontal (even numbers). Each set of spots on the image are spaced by 10 μm from center to center. FIG. 1B shows on-tissue SEM images of 4 different ablation craters created using a Bruker MALDI timsTOF flex with a total laser power of 12.5% and 25 shots. The average ablation crater size was determined to be 4.2±0.2 μm by 2.5±0.2 μm. SEM images were acquired with a magnification of 25,000×. Distance measurements were performed using the instrument software.

FIG. 2 shows the MALDI timsTOF flex instrumental parameters for positive (top) and negative (bottom) ion mode measurements.

FIG. 3 shows the structures and associated physical properties of ACA, DMACA, MACA, and TMACA. The rapifleX vacuum referenced in this figure exposes the compounds to a pressure of approximately 5E-7 mbar.

FIG. 4 shows the structures and associated physical properties of conventional dual polarity matrices for high spatial resolution MALDI IMS of lipids. The rapifleX vacuum referenced in this figure exposes the compounds to a pressure of approximately 5E-7 mbar.

FIG. 5 shows MALDI IMS ion images of lipids in negative ion mode with a pixel size of 10 μm. DMACA showed the highest ion intensity compared to ACA and MACA for phospholipids.

FIG. 6 shows the average mass spectra using ACA, MACA, and DMACA from approximately 38,000 pixels collected from sagittal mouse brain tissue sections (hippocampus region). All three mass spectra share the same intensity scale.

FIG. 7 shows the MALDI ion images of a known in-source fragment of a sphingolipid. Left image: ACA matrix candidate; center image: MACA matrix candidate; right image: DMACA matrix candidate.

FIG. 8 shows the average mass spectra from the hippocampus region of a mouse brain highlighting the ganglioside mass range. Gangliosides are known to easily fragment during the MALDI process, observed as an increase in GM1 signal (red arrow) compared to GD1 (blue arrow). GD1 is known to be more abundant than GM1 when analyzed by LC-MS in murine brain and ratio closer to 1 are more accurate to actual total ganglioside content when performing MALDI IMS.

FIG. 9 shows MALDI IMS of lipids from 10 μm thick serial sections of a sagittal mouse brain section in negative ion mode, using sublimated DMACA, ACA, DAN, and DHA at 10 μm pixel sizes using a Bruker timsTOF fleX. Corresponding average mass spectra can be found in FIG. 10. In all signals but one (PS 40:6 observed at m/z 834.529), DMACA and ACA performances were similar or superior to both DAN and DHA for phospholipid analysis at high spatial resolution. A post-acquisition H&E-stained image from the DMACA experiment is displayed in the middle for histological reference.

FIG. 10 shows a portion of the average MALDI mass spectrum from data shown in FIG. 9, displaying the higher signal intensities generated using DMACA for phospholipids when compared to other matrices. This increase in signal at 10 μm pixel size amounts to about 2-to-3-fold for most phospholipids except for high-intensity lipids such as 885.549, which only showed a marginal increase using DMACA. All average spectra are on the same intensity scale for comparison purposes.

FIG. 11 depicts human kidney tissue sprayed with DAN and DMACA at their respective optimal laser powers as labeled in the image. At a total laser power of 20%, optimal for DAN, DMACA produces a higher quality MALDI IMS measurement, which results both in improved signal intensity and a lower amount of visible delocalization. At 12.5% total laser power, DAN can no longer achieve sufficient desorption/ionization for detecting lipids, while DMACA still offers high-quality IMS with ion intensity similar to that of DAN at 20% total laser power.

FIG. 12 shows MALDI IMS ion images of the serial section of rat brain hippocampus region collected with a 20 μm pixel size, particularly highlighting the unique distribution of ganglioside GM1 at m/z 1544.86 in red, GD1 at m/z 1835.96 in blue, and GT1 at m/z 2127.06 in green with all four new matrices. ACA presented the lowest amount of in-source fragmentation, leading to a more blueish image dominated by GD1 compared to the other matrices where GM1 dominated with the red color.

FIG. 13 shows the MALDI IMS ion images of a rat brain hippocampus collected with a 20 μm pixel size with either DHA (top) or ACA (bottom) after ammonium sulfate spray. ACA showed a ratio of ganglioside between GM1 and GD1 closer to what is observed using total brain extract and LC-MS. This may in part be due, without being bound by theory, to the ability of ACA to minimize in-source fragmentation, which typically is observed to lead to GD species fragmenting into GM like fragments.

FIGS. 14-15 show the MALDI MS spectra of peptide and protein standards using CHCA (red) or DMACA (blue), highlighting the ability of DMACA to desorb and ionize peptides and proteins in a similar fashion to CHCA a standard MALDI matrix for peptide analysis.

FIG. 16 depicts ACA without (left) and with (right) trifluoroacetic acid (TFA) added to the matrix solution. Slight change in coloration was observed as early as 1 min after addition of TFA, with full orange coloration being visible after a few hours. The image of FIG. 12 depicts the color of the ACA matrix solution 24 hours after TFA was added.

FIG. 17 depicts MALDI IMS of gangliosides in the hippocampus region of a sagittal rat brain section in negative ion mode at 20 μm pixel size, using either ACA (top) or DHA (bottom) at 15.5% total laser power. The average spectra highlight the signal boost for gangliosides using ACA over DHA and suggest, without being bound, a better detected ratio of GM1 to GD1. Ion images for four typical ganglioside species show the higher quality measurements obtained through ACA and its ability to better resolve quadra-sialic acid ganglioside. The mass spectra are at the same intensity scale.

FIG. 18 provides MALDI IMS of gangliosides from a sagittal mouse brain tissue section, collected with a 20 μm pixel size in negative ion mode. Among the 10 examined gangliosides, all show high intensity and image quality with ACA over DHA using the same decoupled salt and matrix spray method.

FIG. 19 shows the SEM image of a sprayed layer of ACA over a human kidney tissue section, illustrating the highly homogenous coating and the sub-micron crystal size typical of all ACAA matrix candidate.

FIG. 20 shows the average MALDI spectrum for DMACA and DAN in positive ion mode, using human kidney tissue as the sample (˜200,000 spectra). The laser power was set to generate an ˜6 μm diameter ablation crater to simulate 10 μm pixel size imaging conditions (total laser power: 20%). For most phospholipid signals, DAN has 2-3 times less absolute signal intensity than DMACA.

FIG. 21 shows the Average MALDI spectrum for both DMACA and DAN in positive ion mode, using human kidney tissue as the sample (˜200,000 spectra). The laser power was set to generate ˜4 μm diameter ablation crater to simulate 5 μm pixel size imaging conditions (total laser power: 12.5%). DAN has at least two orders of magnitude less signal intensity than DMACA for all phospholipid signals. This type of low laser power experiment is not only beneficial for 5 μm pixel size IMS, but is also necessary to minimize tissue damage for the integration of MALDI IMS with other modalities' measurement processes in multimodal workflows.

FIG. 22 shows the average MALDI spectrum for both DMACA and DAN in negative ion mode, using human kidney tissue as the sample, as seen in FIG. 11B. The laser power was set to generate an ˜6 μm diameter ablation crater to simulate 10 μm pixel size imaging conditions (total laser power: 20%). DAN can be seen as having equal or lesser absolute signal intensity than DMACA, especially in the m/z 700 range.

FIGS. 23A-23B shows MALDI IMS of a human kidney tissue section at 10 μm pixel size. FIG. 23A depicts human kidney tissue sprayed with DAN at left and sprayed with DMACA at right in negative ion mode using a total laser power of 20%. (250,000 pixels with base peak with an intensity of 8.5E4.) FIG. 23B depicts the high spatial resolution (10 μm raster) MALDI IMS ion images of a human kidney using sprayed DAN (left) and DMACA (right) in negative ion mode at a total laser power of 12.5%. (50,000 pixels with B.P. at 4E4).

FIG. 24 shows the average MALDI spectrum for both DMACA and DAN in negative ion mode, using human kidney tissue as the sample, as seen in FIG. 11B. The laser power was set to generate ˜4 μm diameter ablation crater to simulate 5 μm pixel size imaging conditions (TLP 12.5%). DAN can be seen as having at least two orders of magnitude less absolute signal intensity than DMACA for all phospholipid signals. This type of low laser power experiment is not only beneficial for 5 μm pixel size IMS, but is also necessary to minimize tissue damage for the integration of MALDI IMS with the measurement processes and methods for other modalities in multimodal workflows.

FIG. 25 shows the high spatial resolution (10 μm pixel size) MALDI IMS of a mouse brain section, using sublimated DHA and DMACA in negative ion mode with a total laser power of 20% (left, ˜9 μm spot size) and 12.5% (right, ˜4.5 μm spot size). Many ions show significantly higher intensity using DMACA, particularly sulfatides (e.g., SHexCer(42:1;O3) in green). (bottom) On-tissue average mass spectra from the above mouse brain MALDI IMS experiment using DHA optimal laser power of 20% total laser power (left) and DMACA optimal laser power of 12.5% total laser power (right). All spectra are to the same absolute scale. DMACA allows for a lower laser power to be used without necessarily compromising ion signal yield, effectively allowing for higher spatial resolution acquisitions to be achieved while maintaining sensitivity.

FIGS. 26A-26B shows the high spatial resolution (10 μm raster) MALDI IMS of a human kidney using sublimated DMACA. FIG. 26A: Positive ion mode. 800,000 pixels with B.P. at 8.0E4. FIG. 26B: negative ion mode. (800,000 pixels with B.P. at 3.2E4).

FIG. 27 shows the MALDI IMS of a human retina with a pixel size of 5 μm in negative ion mode. The red, teal, and blue line correspond to sub cellular lipid localization within the photoreceptor layer sitting right above the RPE in yellow.

FIG. 28 shows the SEM image of a sublimated layer of DMACA over a human kidney tissue section, illustrating the highly homogenous coating and the 250 nm average crystal size.

FIG. 29 provides on-tissue SEM images of a sublimated layer of DMACA (0.22 μg/mm²) before (left) and after annealing. Annealing at 100° C. for 30 s allows DMACA to form 200-500 nm crystals, concurrently increasing the signal intensity of phospholipids in both polarities by two to three-fold.

FIG. 30 is a photograph of an ITO slide after deposition of 0.22 μg/mm² of DMACA and annealing for 30 seconds. The two black lines delimit the region onto which the matrix was sublimated. The red circle highlights the region of the slide that received thermal treatment and the white circle the region that was not exposed to heat.

FIG. 31 shows average MALDI mass spectra from rat kidney tissue after (top) matrix spraying, (middle) sublimation, and (bottom) sublimation followed by annealing, in positive ion mode. Sublimation followed by annealing generated the highest signal intensity for polar lipids. All spectra use the same absolute scale.

FIG. 32 shows average MALDI mass spectra from rat kidney after (top) matrix spraying, (middle) sublimation, and (bottom) sublimation followed by annealing, in negative ion mode. Sublimation followed by annealing generated the highest signal intensity for polar lipids. All spectra use the same absolute scale.

FIG. 33 provides MALDI IMS measurements of 6 μm thick serial sections of a human kidney sample at 5 μm pixel size, using sublimated DMACA as the matrix in both positive (left) and negative (right) ion mode. Both positive and negative modes highlight the complex structure of the human kidney, with the cortex region at the top, where glomeruli can be visualized in red, and the medulla region at the bottom. A post-acquisition PAS stain of the negative mode experiment is presented in the middle for histological reference. Total ion current normalized.

FIG. 34 shows Periodic Acid-Schiff (PAS) staining of a human kidney section after MALDI IMS measurement (see FIG. 33) in positive ion mode. The top part of the section is the cortex region with many glomeruli (red arrows). The bottom part is the medulla, which is composed of loops of Henley and vasculature.

FIG. 35 shows 5× magnification of a set of glomeruli from FIG. 4, along with the same section's post-acquisition PAS stain showing molecular and anatomical heterogeneity in the glomeruli along with the absence of burn marks from laser irradiation.

FIG. 36 MALDI IMS average spectra from mouse brain in negative ion mode comparing two ACAA matrices on serial sections using identical laser power. A 3-4 fold increase in ion signal can be observed when using MAPCA as opposed to DMACA. This is attributed to both a higher extinction coefficient at 355 nm and a more closely located absorption maxima at 359 nm for MAPCA as opposed to 330 nm for DMACA.

FIG. 37 MALDI IMS of mouse brain serial sections using different ACAA matrices at 10 μm spatial resolution in negative ion mode. When using identical laser settings MAPCA there is an overall increase in ion intensity over DMACA, particularly in the white matter as shown in green.

FIG. 38 A mosaic of 200 lipid ion images generated using MALDI IMS collected with a pixel size of 10 μm in negative ion mode.

FIG. 39 MALDI IMS of a rat brain cerebellum collected with a pixel size of 5 μm in negative ion mode using MAPCA. The increased sensitivity provided by MAPCA allows the visualization on never fiber tract in yellow extending toward Purkinje cells in orange as seen in the 5× zoomed image.

FIG. 40 Scanning electron microscopy image after MALDI IMS at different total laser power at 10 μm spatial resolution. MAPCA allows a steep reduction in total laser power from a typical 4.5% for imaging with a pixel size of 5 μm to 2.4%.

FIG. 41 MALDI IMS of a rat cerebellum collected with a pixel size of 3 μm using MAPCA in negative ion mode. Purkinje cell's nucleus can be seen as an absence of signal from PI(38:5) in red forming circular holes between the granular layer and cortex region of the rat cerebellum (white arrows).

FIG. 42 MALDI IMS of a rat cerebellum collected with a pixel size of 5 μm using MAPCA in negative ion mode of the same tissue section as FIG. 41. Purkinje cell's nucleus can also be seen as an absence of signal from PI(38:5) in red forming circular holes between the granular layer and cortex region of the rat cerebellum with less definition than its 3 μm counterpart.

FIG. 43 MALDI IMS of a human optic nerve at 3 μm spatial resolution using MAPCA in negative ion mode. The transition between from purple closer to the brain indicating high amount of m/z 902 over m/z 904 which transition to pink as we get closer to the retina expected of a ratio closer to 1:1. This than changes to red in the region known to be a demyelinated region of the optic never next to the retina.

FIG. 44 shows MALDI IMS of a rat kidney collected with a pixel size of 3 μm in negative mode (top) and positive mode (middle). Same section H&E after MALDI IMS in positive mode (bottom). 3 μm spatial resolution enables the better visualization of glomeruli heterogeneity along with obtaining more defined tubular boundaries. ˜1,000,000 pixels or 20 h of acquisition. The base peak had an intensity of 7.0E3 arbitrary units negative and 2.2E4 arbitrary units positive

FIG. 45 shows a high spatial resolution (3 μm pixel size) MALDI IMS of a human kidney (VAN30) using sublimated/annealed MAPCA in positive ion mode. MAPCA allows for 3 μm spatial resolution detection of phosphatidylcholine, sphingomyelin, and carnitine in positive mode without major loss in signal. Proximal tubule in green. Descending limb in yellow. Ascending limb in purple and pink. Glomeruli in red/blue. ˜1,100,000 pixels or 22 h of acquisition. The base peak had an intensity of 2.5E4 arbitrary units.

FIG. 46 provides a MALDI IMS mosaic of 16 individual ion images out of ˜50 obtained with a pixel size of 3 μm using a human kidney tissue section from FIG. 5 dataset (ion from FIG. 45 not included). Ion images show the heterogenous distribution of lipids found in human kidney and show case the image quality obtainable at 3 μm spatial resolution from a timsTOFflex without any instrumental hardware modifications.

### DETAILED DESCRIPTION

In some aspects of the present disclosure, there are provided compounds and compositions for use in the analysis of a sample by matrix-assisted laser desorption ionization (MALDI) mass spectrometry. Methods of use for the compounds and compositions disclosed herein are also provided.

The present disclosure provides, in some aspects, compositions and methods related to aminated cinnamic acid derivatives for high spatial resolution MALDI imaging mass spectrometry (IMS) of metabolites and lipids along with capability to ionize peptides and proteins. In certain aspects of the disclosure, there are provided methods of use for sublimated aminated cinnamic acid analogues (ACAA) followed by matrix annealing for 5 μm spatial resolution dual polarity MALDI IMS of lipids with high sensitivity and low toxicity compared to previous matrices. In some embodiments, use of the presently disclosed methods facilitates lower than 5 μm spatial resolution dual polarity MALDI IMS, such as 3 μm spatial resolution. The present disclosure provides, in some aspects, methyl 4-aminophenyl-2-cyanoacrylate (MAPCA), 3-(4-amino-phenyl)-2-cyanoacrylic acid (APCA), and 2,2′-diamino-4,4′-stilbenedicarboxilic methyl ester (ASCM) as candidates for dual polarity matrix assisted laser desorption/ionization (MALDI) of biomolecules from thin tissue section for high spatial resolution imaging mass spectrometry (IMS). The physical properties of these molecules, especially their vacuum stability and the optical properties, make them improved MALDI matrices.

The MALDI matrices of the present disclosure may have improved or favorable sensitivity towards target analytes; may have improved or favorable vacuum stability; may have favorable extinction coefficients (e.g., 355 nm); may be sprayed or otherwise deposited using non-carcinogenic or otherwise favorable solvents; may be able to be sublimated; may be economically attractive, such as inexpensive or comprise inexpensive components; may have low reported toxicity in humans; or any combination of the above features. In some embodiments, the matrices disclosed herein are less toxic and have better vacuum stability over MALDI matrices presently in common use. In some embodiments, the matrices of the present disclosure require less laser energy to ionize analyte molecules, and therefore have improved sensitivity to analyte molecules. The compositions disclosed herein have been shown, such as described in the sections above and below, to be effective for analytes that are metabolites, lipids, peptides, and proteins. In some embodiments, use of the matrices disclosed herein is associated with MALDI methods that require fewer laser shots, which may prolong the life of the laser used for the MALDI experiment. In some embodiments, use of the matrices disclosed herein is associated with MALDI methods that have a reduced laser beam diameter at the sample surface, which may improve the spatial resolution imaging of the method.

In some aspects, provided herein are methods for the use of sublimated aminated cinnamic acid analogues (ACAA). ACAA compounds may be candidates to replace common MALDI matrices for dual polarity MALDI IMS of lipids due to their reduced laser power requirement, their low toxicity, high sensitivity, and their near-perfect vacuum stability. This gives ACAA compounds an edge for both high spatial resolution MALDI IMS and high specificity imaging experiments (e.g., ion mobility or FTMS) that require longer acquisition times, often at elevated source temperatures (150° C.) that can lead to loss of signal for commonly used matrices that degrade or sublime from the tissue surface over time. In some aspects, described herein is a new sublimation protocol for high spatial resolution MALDI IMS which was developed by combining an ultra-thin amorphous matrix layer followed by an annealing step to induce crystallization increasing signal by 3-4 fold without inducing detectable analyte delocalization. The use of 4-aminocinnamic acid for 5 μm spatial resolution MALDI IMS from Alzheimer's afflicted human brain tissue showing the intricate molecular distribution of gangliosides around 0-amyloid deposits has been demonstrated. The use of 4-(dimethylamino)cinnamic acid for dual polarity 5 μm spatial resolution phospholipid MALDI IMS of human kidney and eye has also been demonstrated. In the case of the human kidney, researchers were able to annotate through exact mass ˜120 lipids in positive mode and ˜175 lipids in negative mode. In some aspects, provided herein are methods for the use of a 2nd generation of ACAA compounds to expand both the molecular coverage and sensitivity by tailoring their absorption band to the wavelength of our MALDI laser (355 nm). This fine-tuning of the absorption band through the addition of selected functional groups allows an additional reduction in laser power leading to a further reduction of the spot size while increasing affinity for peptides, glycans, and proteins. In some embodiments, this 2nd and 3rd generation of ACAA compounds may be applied to a human tissue bank comprised of kidney, pancreas, brain, and eye.

Details of these aspects and more are provided below.

### I. MATRIX-ASSISTED LASER DESORPTION IONIZATION IMAGING MASS SPECTROMETRY

In one aspect, the present disclosure provides new compounds and methods for the analysis of samples by matrix-assisted laser desorption ionization imaging mass spectrometry (MALDI IMS).

In some embodiments, the present methods comprise obtaining a sample comprising an analyte. In some embodiments, the analyte may degrade or decompose in the presence of elevated temperature. The analyte may, in some embodiments, be a peptide or a protein. In some embodiments, the analyte is a lipid, such as a phospholipid or a ganglioside. In some embodiments, the analyte is an organic molecule. In other embodiments, the analyte is an inorganic molecule. In some embodiments, the analyte may be a toxin or a toxic chemical. In some embodiments, the analyte is a metabolite. In some embodiments, the analyte is a peptide. In some embodiments, the analyte is a protein. In some embodiments, the analyte is a glycan. In some embodiments, the analyte is a molecular probe. In some embodiments, the analyte is an antibody. In some embodiments, the analyte comprises a photocleavable tag with a known mass. In such embodiments, the detection of the analyte is equivalent to the detection of the mass of the photocleavable tag. The presently disclosed methods may be useful in immunohistochemistry assays, experiments, or studies.

In some embodiments, the identity of the analyte may be known prior to the execution of the method. In such embodiments, the methods may be used to confirm the presence of the known analyte. In other embodiments, the identity of the analyte may not be known prior to the execution of the method. In such embodiments, the methods may be used to identify the identity of the analyte.

In some embodiments, the analyte may be a macromolecule. In some embodiments the analyte may be a polymer, such as a biopolymer. In some embodiments, the analyte may be a molecular probe used as a mass-tag for MALDI-immunohistochemistry. In some embodiments, the analyte may be a microbe or relevant to microbiology. In some embodiments, the analyte may be a disease marker, or relevant to the diagnosis of a disease. The analyte molecule or molecules may be found in a solution, in solid form, or as a component of another material, such as a tissue. Any off the neat analyte, the analyte in solution, or the analyte as a component of another may be referred to herein as the “analyte sample.”

To facilitate deposition of the compounds (e.g., matrices) of the present disclosure, in some embodiments the compounds of the present disclosure may first be dissolved in a solvent. In some embodiments, the present methods use solvents wherein the matrix of use in the methods is sufficiently or acceptably soluble in the solvent, as determined by the practitioner. In some embodiments, the solvent is an organic solvent. In other embodiments, the solvent is an aqueous solvent. Non-limiting examples of organic solvents include acetonitrile, methanol, acetone, chloroform, dimethylformamide, tetrahydrofuran or other ethers, piperidine or other heterocycloalkyl compounds, ethanol or other alcohols such as propanol, or ethyl acetate or other esters. In some embodiments, the solvent is acetone. In some embodiments, the solvent is substantially tetrahydrofuran. In some embodiments, the solvent is substantially methanol. In some embodiments, the solvent is substantially acetonitrile. In some embodiments, the solvent comprises a mixture of solvents. In some embodiments, the solvent is a mixture of acetonitrile and tetrahydrofuran. In some embodiments, the solvent may comprise dimethylformamide. In some embodiments, the solvent comprises a mixture of at least one organic solvent and water, such as acetonitrile and water or methanol and water. In some embodiments, the solvents may comprise an acid. In some embodiments, the solvents may comprise a base.

In some embodiments, the presently disclosed methods involve spraying or sublimation, which is the gold standard of high spatial resolution MALDI IMS, of the cinnamic acid derivatives disclosed herein. Either spraying or subliming methods may involve the use of techniques, apparatus, or experimental set-ups that have been shown to demonstrate preferable results in other contexts, such as with other known MALDI or MALDI IMS matrices. Spraying of the matrices may, for example, be accomplished with a commercially available sprayer (see Examples for more details). Variable settings of a commercially available sprayer used in depositing matrices or compounds of the present (e.g., nozzle temperature, temperature of heated stage, puck temperature, number of passes, flow rate, velocity or spray speed, spacing or track spacing, spray pattern, pressure, including N₂ pressure), and/or nozzle distance) may be selected by the skilled artisan to achieve successful deposition.

In some embodiments, the analyte sample is on a surface when the spraying occurs. Therefore, in such embodiments, the spraying described above and elsewhere in the present application provides a surface with a sprayed matrix-analyte sample composition. In some embodiments, the surface may be heated. In some embodiments, the surface is about 50° C., about 60° C., about 70° C., about 80° C., about 90° C., about 100° C., about 110° C., about 120° C., about 130° C., about 140° C., about 150° C., or any range derivable therein. In some embodiments, the tray is heated to between about 50° C. and about 120° C., such as about 60° C. In some embodiments contemplated for the instantly claimed methods, the surface is heated to 120° C.

In some embodiments, the spraying involves the use of a nozzle. In some embodiments, the nozzle is heated. In some embodiments, the nozzle is about 50° C., about 60° C., about 70° C., about 80° C., about 90° C., about 100° C., about 110° C., about 120° C., about 130° C., about 140° C., about 150° C., or any range derivable therein. In some embodiments, the nozzle temperature is 50° C. In some embodiments, the nozzle is 60° C. or 70° C. In some embodiments, the nozzle is 120° C.

In some embodiments, the methods comprise sublimating a compound of the present disclosure on the analyte sample. The analyte sample may be located on a surface when the sublimation occurs. Therefore, in such embodiments, the sublimation described above and elsewhere in the application provides a surface with a sublimated matrix-analyte sample composition. The amount of compound sublimated on the analyte sample may vary. About 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, or any range derivable therein may be deposited on the analyte sample. The sublimation may occur at an elevated temperature. In some embodiments, the sublimation occurs at about 80° C., about 90° C., about 100° C., about 110° C., about 120° C., about 130° C., about 140° C., about 150° C., about 160° C., about 170° C., about 180° C., about 190° C., about 200° C., about 210° C., about 220° C., about 230° C., about 240° C., about 250° C., about 260° C., about 270° C., about 280° C., or any range derivable therein. In some embodiments, the sublimation occurs at between about 150° C. to about 210° C., such as about 180° C. or about 160° C. In some embodiments, the sublimation occurs at between about 100° C. to about 140° C., such as at about 110° C. or about 130° C. In some embodiments, the sublimation occurs at reduced pressure, such as about 10 mbar, about 20 mbar, about 30 mbar, about 40 mbar, about 50 mbar, about 60 mbar, about 70 mbar, about 80 mbar, about 90 mbar, about 100 mbar, about 110 mbar, about 120 mbar, about 130 mbar, about 140 mbar, about 150 mbar, about 160 mbar, about 170 mbar, about 180 mbar, about 190 mbar, about 200 mbar, about 250 mbar, about 300 mbar, about 350 mbar, about 400 mbar, about 450 mbar, about 500 mbar, or any range derivable therein. In some embodiments, the sublimation occurs at a pressure of from about 30 mbar to about 60 mbar, such as about 40 mbar or about 50 mbar. In some embodiments, the sublimation occurs at between about 25 mbar and about 500 mbar. In some embodiments, the sublimation occurs at between about 100 mbar and about 200 mbar, such as at about 110 mbar. The sublimation of the compounds disclosed herein on the analyte sample is performed for a particular time period, known as the “sublimation time period.” The sublimation time period is any amount of time that allows an instantly claimed method to completely sublime and, optionally, deposit on a surface. The sublimation time period may be about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, or any range derivable therein. The sublimation period may be from about 5 minutes to about 25 minutes, such as about 10 minutes or about 20 minutes. The sublimation may be carried out through means known to someone of skill in the art. An example of an apparatus that may be used to carry out the sublimation may be found in Hankin et al., 2007.

In some embodiments, the matrix compounds and analyte (also referenced herein as analyte-matrix co-crystal composition) may be heated (“annealed”) following deposition according to any of the methods described above. In some embodiments, an analyte-matrix co-crystal composition formed by sublimation is heated prior to irradiation with a laser. The temperature that the analyte-matrix co-crystal composition is heated to is referenced herein as the annealing temperature. In some embodiments, the annealing temperature is about 50° C., about 60° C., about 70° C., about 80° C., about 90° C., about 100° C., about 110° C., about 120° C., about 130° C., about 140° C., about 150° C., about 160° C., about 170° C., about 180° C., about 190° C., about 200° C., about 210° C., about 220° C., about 230° C., about 240° C., about 250° C., about 260° C., about 270° C., about 280° C., about 290° C., about 300° C., or any range derivable therein. In some embodiments, the annealing temperature is about 70° C., about 80° C., about 90° C., about 100° C., about 110° C., about 120° C., or about 130° C. In some embodiments, the annealing temperature is about 100° C. The length of time that the analyte-matrix co-crystal composition is heated for is referenced herein as the annealing time. In some embodiments, the annealing time is about 10 seconds, about 15 seconds, about 20 seconds, about 25 seconds, about 30 seconds, about 35 seconds, about 40 seconds, about 45 seconds, about 50 seconds, about 10 seconds, about 60 seconds, about 70 seconds, about 80 seconds, about 90 seconds, about 100 seconds, about 120 seconds, or any range derivable therein. In some embodiments, the annealing time is about 1 minute, about 2 minutes, about 3 minutes, about 4 minutes, about 5 minutes, about 6 minutes, about 7 minutes, about 8 minutes, about 9 minutes, about 10 minutes, about 15 minutes, about 20 minutes, about 25 minutes, about 30 minutes, or any range derivable therein. In some embodiments, the annealing time is between about 15 seconds and about 45 seconds. In some embodiments, the annealing time is about 30 seconds. In some embodiments, the annealing time is between about 10 seconds and about 20 seconds. In some embodiments, the annealing time is about 15 seconds.

In some embodiments, the depositing of the matrix compounds may provide for the formation of advantageously small matrix crystals or analyte-matrix co-crystals, such as sub-micron matrix crystals or co-crystals. In some embodiments, the presently disclosed cinnamic acid derivatives form highly homogeneous crystals, co-crystals, crystal layers, or co-crystal layers. In some embodiments, the crystals or co-crystals are smaller than the wavelength of the MALDI laser. In some embodiments, the crystals or co-crystals are less than 5000 nm. In some embodiments, the matrix crystals or co-crystals described herein are about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1000 nm, about 1500 nm, about 2000 nm, about 2500 nm, about 3000 nm, about 3500 nm, about 4000 nm, about 4500 nm, about 5000 nm, or any range derivable therein. In some embodiments, the matrix crystals or co-crystals of the present disclosure are about 250 nm or about 200 nm, which are typically hard to obtain using standard sublimation process where micron scale (1-2 μm) crystals are the norm. In some embodiments, the matrix crystals or co-crystals are about 500 nm. In some embodiments, the characteristics of the crystals or co-crystals of the presently disclosed matrices, such as small size or ability to generate finer particles, allow for more ion to be produced.

The present methods provide analyte-matrix co-crystals. The present methods comprise, in some embodiments, spraying, subliming, or spotting the matrix compounds disclosed herein, on the analyte sample on a surface, as described above, to form an analyte-matrix crystal. The present methods provide analyte-matrix co-crystals that in some embodiments are about 0.05 μg/mm², about 0.10 μg/mm², about 0.12 μg/mm², about 0.14 μg/mm², about 0.16 μg/mm², about 0.18 μg/mm², about 0.20 μg/mm², about 0.21 μg/mm², about 0.22 μg/mm², about 0.23 μg/mm², about 0.24 μg/mm², about 0.25 μg/mm², about 0.26 μg/mm², about 0.27 μg/mm², about 0.28 μg/mm², about 0.29 μg/mm², about 0.3 μg/mm², about 0.32 μg/mm², about 0.34 μg/mm², about 0.36 μg/mm², about 0.38 μg/mm², about 0.40 μg/mm², about 0.42 μg/mm², about 0.48 μg/mm², about 0.50 μg/mm², about 0.55 μg/mm², about 0.60 μg/mm², about 0.65 μg/mm², about 0.70 μg/mm², about 0.75 μg/mm², about 0.80 μg/mm², about 0.85 μg/mm², about 0.90 μg/mm², about 0.95 μg/mm², about 1.0 μg/mm², or any range derivable therein. In some embodiments, the analyte-matrix co-crystals are from about 0.20 μg/mm² to about 0.30 μg/mm², such as about 0.25 μg/mm².

In some embodiments, the present methods provide for high spatial resolution MALDI or MALDI IMS. In some embodiments, use of the cinnamic acid derivatives as presently disclosed results in a reduction in in-source fragmentation or a reduction in laser power necessary to carry out the method in comparison to known methods. In some embodiments, the laser spot size on the target is reduced. The present methods may in some embodiments reduce false lipid identification. In some embodiments, the cinnamic acid derivatives of the present invention offer a greater sensitivity for phospholipids at laser power used in known methods and significantly reduce laser power, which enables higher spatial resolution experiments with minimal loss in ion signal.

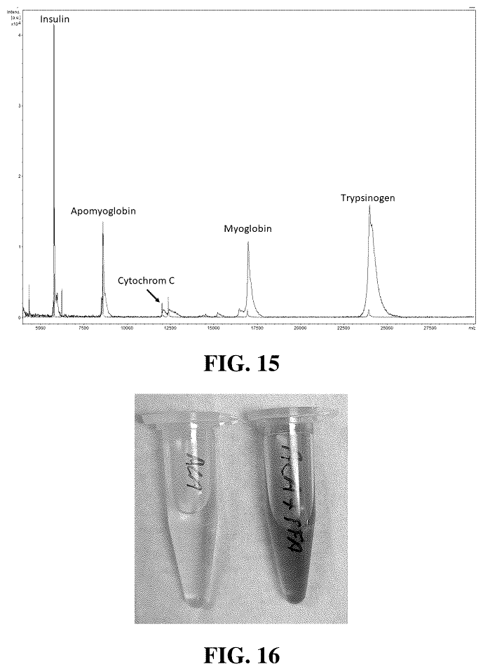

In some embodiments, the sublimation of the presently disclosed cinnamic acid derivative matrices (e.g., DMACA) generate an on-tissue thin film over a human tissue (e.g., a human kidney biopsy or a human eye). In some embodiments, the present methods allow one to achieve a 10 μm spatial resolution or 5 μm spatial resolution, such as shown in FIG. 15 and FIG. 16. In some embodiments, the present methods provide for 5 μm spatial resolution of the inter tubule space in a human kidney sample. The present methods also allow for the observation of intra-glomeruli heterogeneity between m/z 750.545 in purple, tentatively identified as PE(O-38:5), and m/z 788.545 in teal, tentatively identified as PS(18:0_18:1), as shown in FIG. 15. In some embodiments, the present methods allow for visualization of the localization of lipids in a tissue, such as the human eye (see FIG. 16). In some such embodiments, the methods can localize lipids at a subcellular level, for example, inside of the photoreceptor layer of the retina (PL). In some embodiments, the methods and matrices will permit the identification of lipids associated with specific subcellular compartments, for example, of a photoreceptor. In some embodiments the methods and matrices may be used to identify which lipids are associated with specific cell types or multicellular tissue structures, such as rod or cone cells.

### II. CINNAMIC ACID DERIVATIVES

The present disclosure provides cinnamic acid derivatives for use as matrices. These cinnamic acid derivatives may also be referred to herein as “matrices of the present invention”, “matrices of the present disclosure”, “compounds of the present invention” or “compounds of the present disclosure” and are shown, for example, above, in the summary of the invention section, and in the claims below. The cinnamic acid derivatives may be made using the synthetic methods according to principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

Compounds of the present invention may contain one or more asymmetrically-substituted carbon, nitrogen, sulfur, or phosphorus atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S or the R configuration. In some embodiments, the present compounds may contain two or more atoms which have a defined stereochemical orientation.

Chemical formulas used to represent compounds of the present invention will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

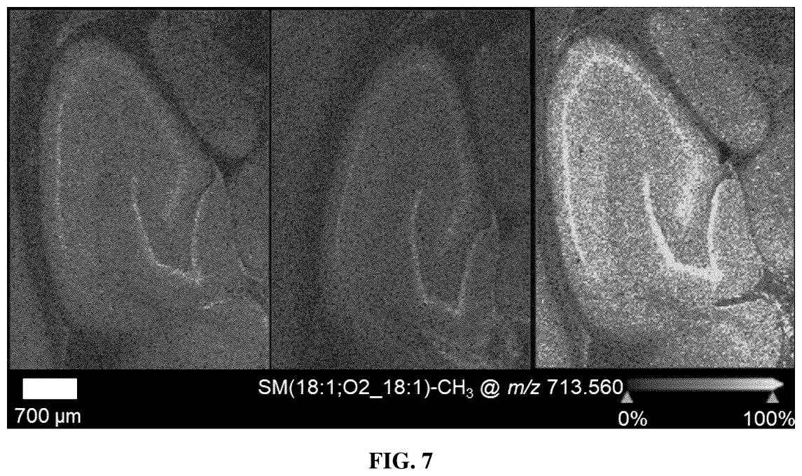

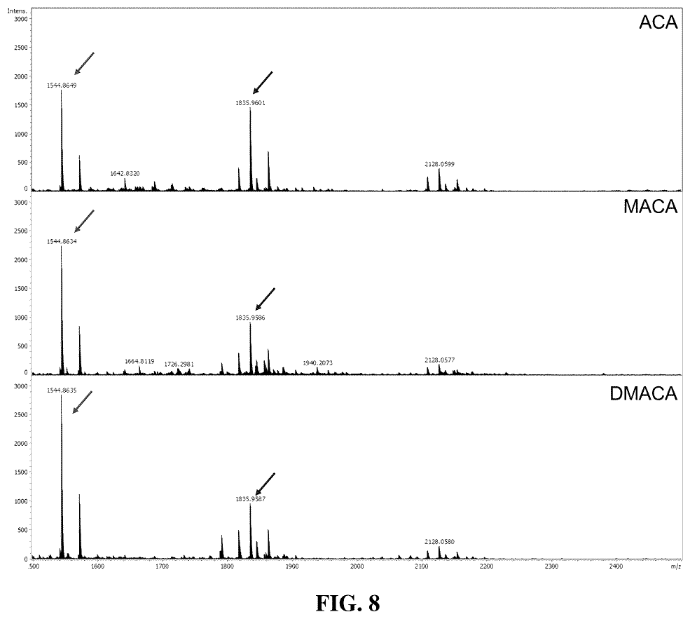

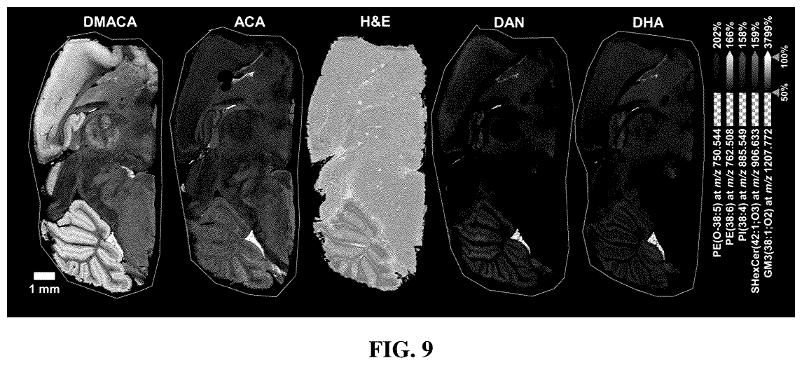

The matrices of the present disclosure have in some embodiments a preferably high extinction coefficient at 355 nm, which, without being bound by theory, allows for more efficient desorption/ionization of the analyte. In some embodiments, matrices of the present disclosure are useful for generating an improved molecular profile for thermally labile molecules. In some embodiments, methods involving the presently disclosed cinnamic acid derivatives provide improved signal or improved signal-to-noise ratio, as shown for example in FIG. 7. In some embodiments, the present methods generate a more accurate ganglioside lipids profile and a reduction in known lipids in-source fragments when compared to the state of the art (see FIG. 8 and FIG. 9). In some embodiments, the presently disclosed cinnamic acid derivative matrices may be classified as soft ionization matrices. The cinnamic acid derivative matrices disclosed herein in some embodiments exhibit high sensitivity to analytes, such as thermally labile molecules, non-limiting examples of which include gangliosides or metabolites. In some embodiments, the present methods provide an improved sensitivity over known methods, such as methods of lipid analysis. In some embodiments, the methods disclosed herein may be particularly useful in the analysis of phospholipids by MALDI, particularly with a laser at 355 nm.

In some embodiments, methyl 4-aminophenyl-2-cyanoacrylate (MAPCA) may be used as a lead matrix due to the proximity of its absorption maxima in solid state giving it the ability to significantly reduce incident laser power leading to lower spot size and lower amount of tissue damage. In certain embodiments, MAPCA may be used as a matrix for multimodal studies using the same tissue section. In certain embodiments, 3-(4-amino-phenyl)-2-cyanoacrylic acid (APCA) and 2,2′-diamino-4,4′-stilbenedicarboxilic methyl ester (ASCM) may be applied to specialized applications where higher laser energy is needed such as forming Metal-Molecule complexes to access alternative ionization pathways.

### III. DEFINITIONS

The When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “thiocarbonyl” means —C(═S)—; “sulfonyl” means —S(O)₂—; and “sulfinyl” means —S(O)—.

In the context of chemical formulas, the symbol “ [symbol] ” means a single bond, “ [symbol] ” means a double bond, and “ [symbol] ” means triple bond. The symbol “ [symbol] ” represents an optional bond, which if present is either single or double. The symbol “ [symbol] ” represents a single bond or a double bond. Thus, the formula

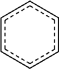

covers, for example,

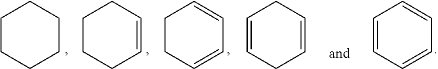

And it is understood that no one such ring atom forms art of more than one double bond. Furthermore, it is noted that the covalent bond symbol “ [symbol] ” when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

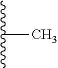

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

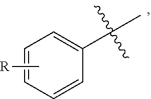

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

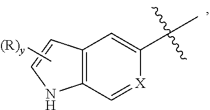

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “alkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C₁₋₄-alkyl”, “C₁₋₄-alkyl”, “alkyl_{(C1-4)}”, and “alkyl_{(C≤4)}” are all synonymous. Except as noted below, every carbon atom is counted to determine whether the group or compound falls with the specified number of carbon atoms. For example, the group dihexylamino is an example of a dialkylamino_{(C≤12)} group; however, it is not an example of a dialkylamino_{(C6)} group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl(ci-6). Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic π system. An aromatic compound or chemical group may be depicted as a single resonance structure; however, depiction of one resonance structure is taken to also refer to any other resonance structure. For example:

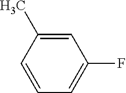

is also taken to refer to

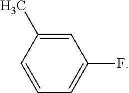

Aromatic compounds may also be depicted using a circle to represent the delocalized nature of the electrons in the fully conjugated cyclic 71 system, two non-limiting examples of which are shown below:

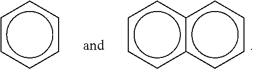

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. If more than one ring is present, the rings may be fused, bridged, or spirocyclic. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

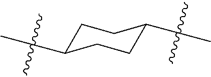 [SMILES: *C1CCC(*)CC1]

is a non-limiting example of cycloalkanediyl group. A “cycloalkane” refers to the class of compounds having the formula H—R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. The configuration of the double bond in an alkenediyl group may be either E or Z. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “alkynyl” refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

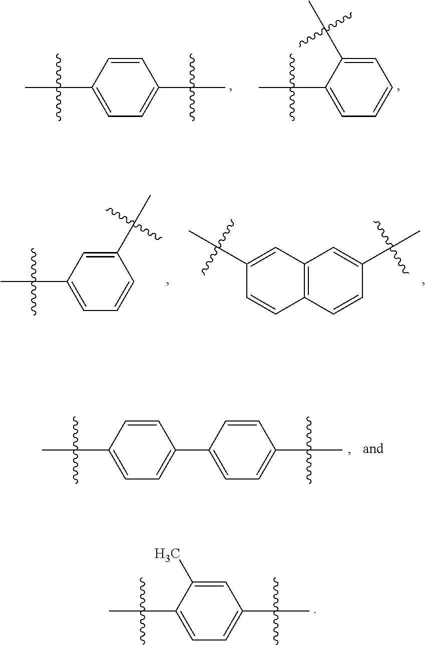

An “arene” refers to the class of compounds having the formula H—R, wherein R is aryl as that term is defined above. Benzene and toluene are non-limiting examples of arenes.

The term “aralkyl” refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl, isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings may be fused, bridged, or spirocyclic. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, tetrahydropyridinyl, pyranyl, oxiranyl, and oxetanyl. The term “N-heterocycloalkyl” refers to a heterocycloalkyl group with a nitrogen atom as the point of attachment. N-pyrrolidinyl is an example of such a group.

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The term “amido” (acylamino), when used without the “substituted” modifier, refers to the group —NHR, in which R is acyl, as that term is defined above. A non-limiting example of an amido group is —NHC(O)CH₃.

The terms “alkylsulfonyl” and “alkylsulfinyl” refers to the groups —S(O)₂R and —S(O)R, respectively, in which R is an alkyl, as that term is defined above. The terms “cycloalkylsulfonyl”, “alkenylsulfonyl”, “alkynylsulfonyl”, “arylsulfonyl”, “aralkylsulfonyl”, “heteroarylsulfonyl”, and “heterocycloalkylsulfonyl” are defined in an analogous manner.

The terms “cycloalkylamino”, “alkenylamino”, “alkynylamino”, “arylamino”, “aralkylamino”, “heteroarylamino”, “heterocycloalkylamino”, and “alkoxyamino” when used without the “substituted” modifier, refers to groups, defined as —NHR, in which R is cycloalkyl, alkenyl, alkynyl, aryl, aralkyl, heteroaryl, heterocycloalkyl, and alkoxy, respectively. A non-limiting example of an arylamino group is —NHC₆H₅. The terms “dicycloalkylamino”, “dialkenylamino”, “dialkynylamino”, “diarylamino”, “diaralkylamino”, “diheteroarylamino”, “diheterocycloalkylamino”, and “dialkoxyamino”, refers to groups, defined as —NRR′, in which R and R′ are both cycloalkyl, alkenyl, alkynyl, aryl, aralkyl, heteroaryl, heterocycloalkyl, and alkoxy, respectively. Similarly, the term alkyl(cycloalkyl)amino refers to a group defined as —NRR′, in which R is alkyl and R′ is cycloalkyl.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

According to the present disclosure, the term “electron-withdrawing group”, “EWG”, or “polar group” references a chemical group that, when in forming a bond to another atom, forms a polar bond with said atom, and further wherein the electron-withdrawing group is more electronegative than said atom such that the dipole moment of the formed polar bond is directed towards the electron-withdrawing group. Non-limiting examples of polar groups include —OH, —F, —Cl, —Br, —I, —CN, —NH₂, —NO₂, —CO₂H, —C(O)H, —C(O)NH₂, —S(O)₂OH, and —S(O)₂NH₂. Additional non-limiting examples of electron-withdrawing groups include —OR, —NHR, —NR₂, —OC(O)R, —CO₂R, —C(O)NHR, —C(O)NR₂, —NHC(O)R, —NHC(O)OR, —NHC(O)NHR, —NHC(O)NHR, wherein each R is independently alkyl_{(C≤12)}, cycloalkyl_{(C≤12)}, or aryl_{(C≤12)} as defined above, provided that the definition laid out above is met. In some embodiments, the electron-withdrawing group may be defined according to the Pauling scale. Preferred examples of electron withdrawing groups may include —Cl and —Br.

When a chemical group is used with the “polar-substituted” modifier, one or more hydrogen atom has been replaced, independently at each instance, by one of the following electron-withdrawing groups: —OH, —F, —Cl, —Br, —I, —CN, —NH₂, —NO₂, —CO₂H, —C(O)H, —C(O)NH₂, —S(O)₂OH, or —S(O)₂NH₂; or —OR, —NHR, —NR₂, —OC(O)R, —CO₂R, —C(O)NHR, —C(O)NR₂, —NHC(O)R, —NHC(O)OR, —NHC(O)NHR, —NHC(O)NHR, wherein each R is independently alkyl_{(C≤12)}, cycloalkyl_{(C≤12)}, or aryl_{(C≤12)} as defined above, provided that the number of carbons of the R group(s) does not cause the resultant compound to exceed atom limitations, and provided that not every hydrogen is so replaced. Non-limiting examples of polar-substituted alkyl groups include —CH₂F, —CHF₂, —CH₂CH₂F, —CHFCH₂F, —CF₂CH₃, —CH₂OH, —CH₂CH₂OH, —CH₂CH₂CH₂NH₂, —CH₂CH₂OH, and —CH(NH₂)CH₂OH.

When a chemical group is used with the “monopolar-substituted” modifier, one and only one hydrogen atom has been replaced by one of the following polar substituents: —OH, —F, —NH₂, —CN, —CO₂H, —CO₂CH₃, —C(O)NH₂, —C(O)NHCH₃, —OC(O)CH₃, —NHC(O)CH₃, —NHC(O)OCH₃, —NHC(O)OCH₂CH₃, —NHC(O)NHCH₃, —NHC(O)NHCH₂CH₃, —S(O)₂OH, or —S(O)₂NH₂. Non-limiting examples of monopolar-substituted alkyl groups include —CH₂F, —CH₂CH₂F, —CHFCH₃, —CH₂OH, —CH₂CH₂OH, —CH(OH)CH₂OH, —CH₂NH₂, —CH₂CH₂NH₂, and —CH(NH₂)CH₃.

A “proton-accepting atom” is any atom that has the ability to act as a Brønsted-Lowry base, often due to the presence of a lone pair or lone pairs on the atom. An atom is considered a proton-accepting atom although, in some molecular or experimental contexts, it may not act as a proton acceptor. Non-limiting examples of proton-accepting atoms are nitrogen, oxygen, sulfur, and phosphorus.

As used herein, the phrase “substantially” means that the composition contains ≤15%, more preferably ≤10%, even more preferably ≤5%, or most preferably ≤1% of another compound(s).

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

An “isomer” of a first compound is a separate compound in which each molecule contains the same constituent atoms as the first compound, but where the configuration of those atoms in three dimensions differs.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Materials and Methods

### Chemicals and Reagents

HPLC grade acetone, acetonitrile (ACN), and tetrahydrofuran (THF) along with 1,5-diaminonaphthalene (DAN), ammonium formate, 2,5-dihydroxibenzoic acid (DHB), and 2,5-dihydroxyacetophenone (DHA; 98+% purity) were acquired from MilliporeSigma (St. Louis, MO). (E)-4-aminocinnamic acid (ACA; 98+% purity), 4-(dimethylamino)cinnamic acid (DMACA; 99% purity), norharman (Nor; 98% purity), and dimethylformamide (DMF) were acquired from Thermo Fisher Scientific (Waltham, MA) or manufactured by TCI or Thermo Sci and acquired from Fisher Scientific (Pittsburgh, PA). All HPLC-grade solvents were purchased from Millipore Sigma (St. Louis, MO). Indium tin oxide-coated microscope slides were purchased from Delta Technology (Loveland, CO). 4-(methylamino)cinnamic acid (MACA) and 4-(dimethylamino)cinnamic methyl ester (TMACA) were provided by the Vanderbilt Institute of Chemical Biology Synthesis Core, Vanderbilt University, Nashville, TN 37232-0412.

### Tissue Sectioning

Mouse and rat brain tissues were purchased from BioIVT (Westbury, NY). Normal portions of human kidney tissue were collected through the Vanderbilt Cooperative Human Tissue Network as part of cancer-related total nephrectomies as part of the Human Biomolecular Atlas Program study. Murine and human tissues were flash frozen and stored at −80° C. (Snyder et al., 2019; Jain et al., 2023). Murine and human tissues were sectioned at 6 μm thickness using a CM3050S cryostat from Leica Microsystems (GmbH, Wetzlar, Germany) and thaw mounted on ITO-coated microscope glass slides. The tissue sections were then washed with 3 or 4 volumes of isotonic ammonium formate (150 mM) for 45 s in each solution and dried using nitrogen gas followed by 20 min in a vacuum desiccator. For long term storage mounted tissue section were vacuum sealed in a plastic bag and stored at −80° C.

### Spectroscopic Measurements

Extinction coefficient measurements were performed at 355 nm using at SpectraMax M2 (Molecular Devices, San Jose, CA). In short, matrices were dissolved in tetrahydrofurane (THF) at 1 mg/mL and diluted to 10 μg/mL for measurement. 1 mL of each matrix solution was pipetted into a cuvette, and absorption at 355 nm was acquired 3 times. For solid state spectra and absorption maxima, each matrix was sublimed into a quartz cuvette using an HTX sublimate (HTX Technologies, Chapel Hill, NC) using 1 mL of 1 mg/mL of the matrix following our standard sublimation protocol below.

### Matrix Application

MALDI matrices were sprayed on the tissue sections using a M5 sprayer with a heated tray from HTX Technologies (Chapel Hill, NC) using either a 2:1 Acetone:ACN with 0.5% DMF or a 1:1:1 Acetone:ACN:THF with 0.5% DMF. The spray nozzle height was changed from 4 cm to 5 cm to accommodate a 1 cm pre-heated puck (130° C.). Preferred spray deposition conditions for individual matrices can be found in Tables 1 and 2. For a first set of exemplary matrices (see Table 1 and Example 2), DHB (40 mg/mL), DHA (30 mg/mL), and DAN (12 mg/mL) were sprayed on tissue for a final surface concentration of 1.36, 0.91, and 0.68 μg/mm² respectively. Additional exemplary spray deposition conditions can be found in Table 2. Other matrices from the aminated cinnamic acid family (e.g., MACA and TMACA) were deposited according the DMACA conditions shown in Table 2. DAN and DMACA (12 mg/mL) were sprayed on tissue according to the parameters shown in Table 2 for a final surface concentration of 0.68 μg/mm² (Dufresne et al., 2023).

TABLE 1
Exemplary spray deposition conditions for MALDI matrices

| | | | Nozzle | Heated | Heated | # | Flow | | Track | | | Nozzle |
|---|---|---|---|---|---|---|---|---|---|---|---|---|
| | | Concentration | Temperature | Stage | Puck | Passes | Rate | Velocity | spacing | | Pressure | distances |
| Matrix | Solvent | (mg/mL) | (° C.) | (° C.) | (° C.) | (count) | (mL/min) | (mm/min) | (mm) | Pattern | (psi) | (mm) |
| DMACA | THF with | 12 | 50 | 60 | 120 | 4 | 0.05 | 1350 | 2 | HH | 10 | 5 |
| | 0.5% | | | | | | | | | | | |
| | DMF | | | | | | | | | | | |
| ACA | 66% | 15 | 50 | 60 | 120 | 4 | 0.05 | 1350 | 2 | HH | 10 | 5 |
| | ACN and | | | | | | | | | | | |
| | 33.5% | | | | | | | | | | | |
| | THF with | | | | | | | | | | | |
| | 0.5% | | | | | | | | | | | |
| | DMF | | | | | | | | | | | |
| DHB | 90% | 20 | 60 | 60 | N/A | 16 | 0.05 | 1350 | 1.5 | HH | 10 | 4 |
| | MeOH | | | | | | | | | | | |
| | and 10% | | | | | | | | | | | |
| | H₂O | | | | | | | | | | | |
| DHB | ACN | 40 | 50 | 60 | 120 | 4 | 0.05 | 1350 | 2 | HH | 10 | 5 |
| | with | | | | | | | | | | | |
| | 0.5% | | | | | | | | | | | |
| | DMF | | | | | | | | | | | |
| DHA | 90% | 25 | 50 | 60 | N/A | 4 | 0.05 | 1350 | 1.5 | HH | 10 | 4 |
| | MeOH | | | | | | | | | | | |
| | and 10% | | | | | | | | | | | |
| | H₂O | | | | | | | | | | | |
| DHA | 66% | 30 | 50 | 60 | 120 | 4 | 0.05 | 1350 | 2 | HH | 10 | 5 |
| | ACN and | | | | | | | | | | | |
| | 33.5% | | | | | | | | | | | |
| | THF with | | | | | | | | | | | |
| | 0.5% | | | | | | | | | | | |
| | DMF | | | | | | | | | | | |
| DAN | 90% | 20 | 70 | 60 | N/A | 6 | 0.05 | 1350 | 3 | HH | 10 | 4 |
| | ACN and | | | | | | | | | | | |
| | 10% H₂O | | | | | | | | | | | |
| DAN | ACN | 15 | 50 | 60 | 120 | 4 | 0.05 | 1350 | 2 | HH | 10 | 5 |
| | with | | | | | | | | | | | |
| | 0.5% | | | | | | | | | | | |
| | DMF | | | | | | | | | | | |

TABLE 2
Additional exemplary spray deposition conditions for MALDI matrices

| | | | | | | | Flow | | | | |
|---|---|---|---|---|---|---|---|---|---|---|---|
| | | | Nozzle | Heated | Heated | # | Rate | Velocity | Track | | |
| | | Concentration | Temperature | Stage | Puck | Passes | (mL/ | (mm/ | spacing | | Pressure |
| Matrix | Solvent | (mg/mL) | (° C.) | (° C.) | (° C.) | (count) | min) | min) | (mm) | Pattern | (psi) |
| DMACA | 1:1:1 | 12 | 40 | 60 | 120 | 4 | 0.05 | 1350 | 2 | CC | 10 |
| | ACN:Acetone:THF | | | | | | | | | | |
| DAN | 1:1 ACN: Acetone | 12 | 40 | 60 | 120 | 4 | 0.05 | 1350 | 2 | CC | 10 |
| DHA | 1:1 ACN: Acetone | 30 | 40 | 60 | 120 | 4 | 0.05 | 1350 | 2 | CC | 10 |
| Ammonium | 2:3 H2O: EtOH | 62 mM | 65 | 60 | N/A | 25 | 0.01 | 1350 | 2 | CC | 15 |
| Sulfate | | | | | | | | | | | |

DMACA: 4-(dimethylamino)cinnamic acid; DAN: 1,5-diaminonaphthalene; DHA: 2,5-dihydroxyacetophenone; ACN: acetonitrile. Other matrices from the aminated cinnamic acid family (e.g., MACA and TMACA) were deposited according the DMACA conditions shown in Table 2.

Sublimated samples were prepared using an in-house prototype sublimation apparatus similar in function to a previously described system (Hankin et al., 2007; Anderson et al., 2023) and similar to the HTX sublimate platform (HTX Technologies, Chapel Hill, NC). In short, the protype sublimation apparatus was filled with a fixed amount of matrix varying between 2.5-20 mg/mL, depending on the matrix, solubilized in acetone. Complete sublimation of the matrix layer was performed at 100-180° C. for 10-15 min at <200 mbar (measured immediately outside the sublimation chamber) onto the sample. Preferred sublimation conditions of matrices can be found in Table 3. Additional exemplary preferred sublimation conditions can be found in Table 4.

TABLE 3
Exemplary sublimation deposition conditions for MALDI matrices

| | | | Amount | | | | | | |
|---|---|---|---|---|---|---|---|---|---|
| | | | placed in | | Temperature | Temperature | | | |
| | | | sublimation | | of | of the | Time of | Crystallization | |
| | Concentration | | vessel | Pressure | sublimation | sample | sublimation | temperature | Crystallization |
| Matrix | (mg/mL) | Solvent | (mL) | (mbar) | (° C.) | (° C.) | (min) | (° C.) | time (s) |
| DMACA | 2.5 | Acetone | 2 | 40-50 | 160 | −78 | 10 | 135 | 20 |
| ACA | 3 | Acetone | 2 | 40-50 | 160 | −78 | 10 | 135 | 20 |
| DAN | 10 | Acetone | 2 | 40-50 | 130 | −78 | 10 | 135 | 15 |
| DHA | 15 | Acetone | 2 | 40-50 | 110 | −78 | 10 | 135 | 10 |

TABLE 4
Additional exemplary sublimation deposition conditions for MALDI matrices

| | | | Amount | | | | | | |
|---|---|---|---|---|---|---|---|---|---|
| | | | placed in | | Temperature | Temperature | | | |
| | | | sublimation | | of | of the | Time of | Crystallization | |
| | Concentration | | vessel | Pressure | sublimation | sample | sublimation | temperature | Crystallization |
| Matrix | (mg/mL) | Solvent | (mL) | (mTorr) | (° C.) | (° C.) | (min) | (° C.) | time (s) |
| DMACA | 3 | Acetone | 2 | ≤20 | 180 | −78 | 15 | 100 | 30 |
| ACA | 6 | Acetone | 2 | ≤20 | 180 | −78 | 10 | 100 | 30 |
| DAN | 10 | Acetone | 2 | ≤20 | 150 | −78 | 10 | N/A | N/A |
| DHA | 20 | Acetone | 2 | ≤20 | 150 | −78 | 10 | N/A | N/A |

Final surface density for deposition conditions shown in Table 3 was about 0.25 μg/mm². Final surface density for deposition conditions shown in Table 4 is provided below in Table 5. Matrix surface concentration was determined by weighing the tissue mounted indium tin oxide (ITO) coated glass slides before and after deposition of the matrices with the M5 sprayer or sublimation. In most cases, up to 3 sequential spray protocol or sublimation on the same slide were required to achieve a measurable weight with less than 10% variation. In some embodiments, thin layers of aminated cinnamic acid derivative (≤0.25 μg/mm²) were sublimated on tissue using a custom-built aluminium apparatus (subliMATE), which enabled the use of dry ice in the cold finger to reach −78° C. and made the sample temperature inside the chamber more consistent during sublimation. The matrices deposited according to the deposition conditions described in Tables 2, 4, and 5 underwent an additional step of thermal annealing where the slides are placed on a hot plate for 30 s at 100° C. before analysis.

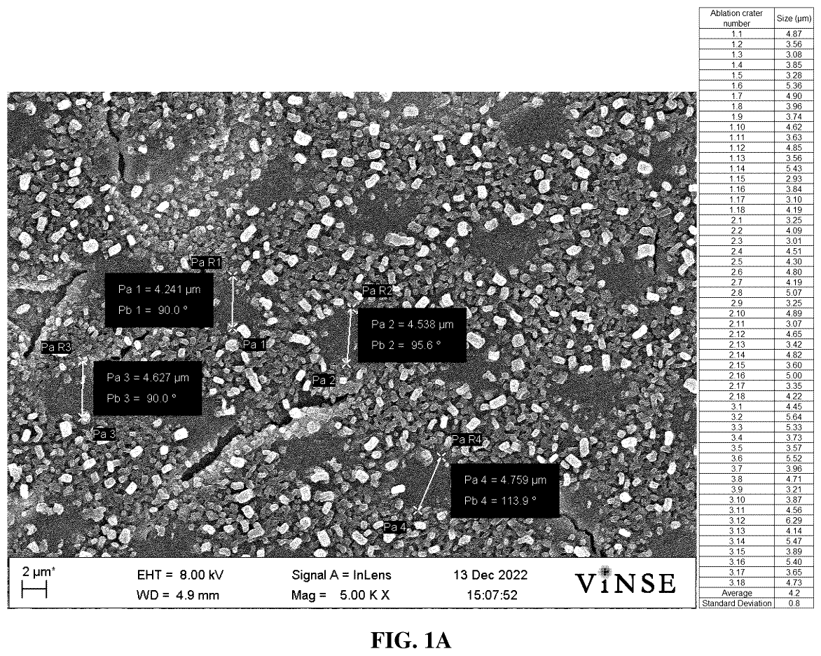

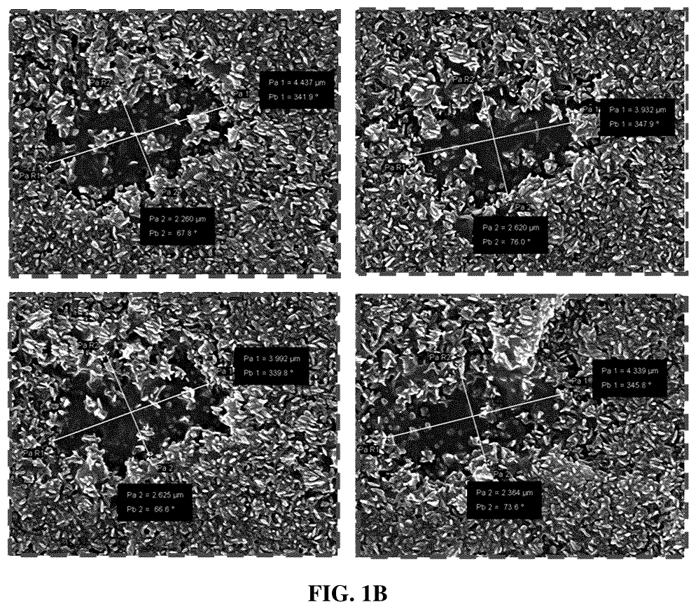

For vacuum stability assessment, ITO slides were weighed before and after sublimation using 15 mg/mL solution. Sublimated slides were then exposed to the vacuum of a Bruker rapifleX mass spectrometer (source vacuum: 2.0E-7 mbar) for at least 24 h. The slides were then weighed again to measure matrix loss from high vacuum exposure (FIG. 1, Table 6). The TMACA matrix layer was lost entirely, as expected from an esterified compound. DHA was also completely sublimed from the substrate, consistent with previous studies that showed DHA matrix layers barely survive 1-2 h in similar high vacuum ion sources (Caprioli et al., 1997).

TABLE 5
On-slide surface density of MALDI matrices of Table
4 after sublimation for high spatial resolution IMS.

| | | Surface density |
|---|---|---|
| Matrix | Organ | (μg/mm²) |
| DHA | Brain | 0.9 |
| DAN | Brain | 0.9 |
| DAN | Kidney | 0.675 |
| ACA | Brain | 0.45 |
| DMACA | Brain | 0.45 |

TABLE 6
Amount of matrix loss after 24 h exposure
in high vacuum (2.0E−7 mbar)

| Amount loss (mg) | ACA | MACA | DMACA | TMACA | DHA |
|---|---|---|---|---|---|
| Average | 0.0 | 0.0 | 0.0 | 1.5 | 1.2 |
| STD | 0.1 | 0.1 | 0.1 | 0.2 | 0.2 |

### Synthetic Methods

Methyl 4-aminophenyl-2-cyanoacrylate (MAPCA or Vandy37; C₁₁H₁₀N₂O₂; MW: 202.2) may be synthesized as shown in Scheme 1 below. In some embodiments, methyl 4-aminophenyl-2-cyanoacrylate may be present in the form of solid orange crystals and may have a λₘₐₓ of 395 nm in THF, a λₘₐₓ solid state equal to 359 nm, an ε at λₘₐₓ of approximately 4.0E4 M⁻¹ cm⁻¹, an F at 355 nm of approximately 1.0E4 M⁻¹ cm⁻¹, and may be vacuum stable for at least 48 h at 5.0E-7 mbar.

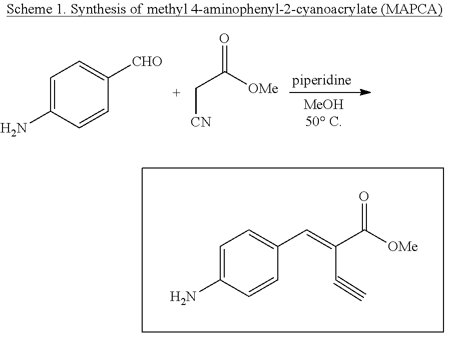

3-(4-amino-phenyl)-2-cyanoacrylic acid (APCA; C₁₀H₈N₂O₂; MW: 188.1) may be synthesized as shown in Scheme 2 below. In some embodiments, 3-(4-amino-phenyl)-2-cyanoacrylic acid may be present in the form of solid yellow crystals and may have a λₘₐₓ of 385 nm in THF, an ε at λₘₐₓ equal to 1.5E4 M⁻¹ cm⁻¹, and is vacuum stable for up to 48 h at 5.0E-7 mbar.

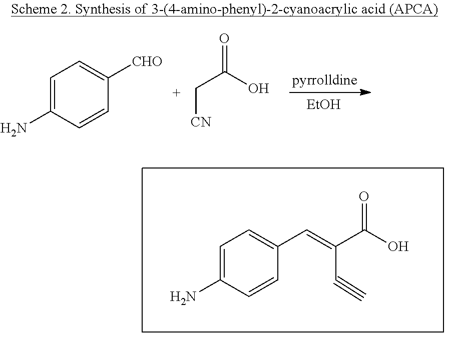

2,2′-Diamino-4,4′-stilbenedicarboxilic methyl ester (ASCM; C₁₈H₁₈N₂O₄; MW: 326.4) may be synthesized as shown in Scheme 3 below. In some embodiments, 2,2′-diamino-4,4′-stilbenedicarboxilic methyl ester may be present in the form of solid yellow crystals and may have a λₘₐₓ of 399 nm in THF, an ε at λₘₐₓ equal to 9.0E3 M⁻¹ cm⁻¹, and may be vacuum stable for up to 48 h at 5.0E-7 mbar.

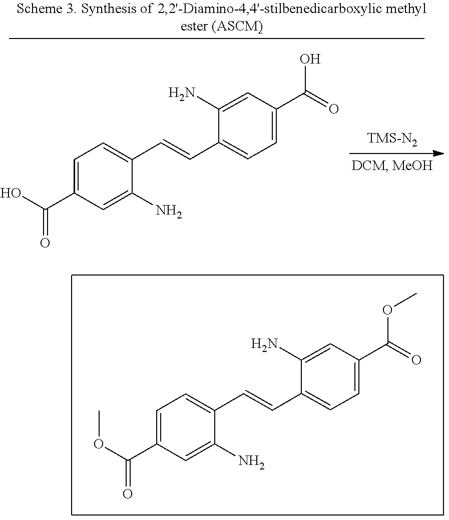 [SMILES: Nc1cc(C(=O)O)ccc1/C=C/c1ccc(C(=O)O)cc1N ; COC(=O)c1ccc(/C=C/c2ccc(C(=O)OC)cc2N)c(N)c1]

### MALDI Imaging Mass Spectrometry

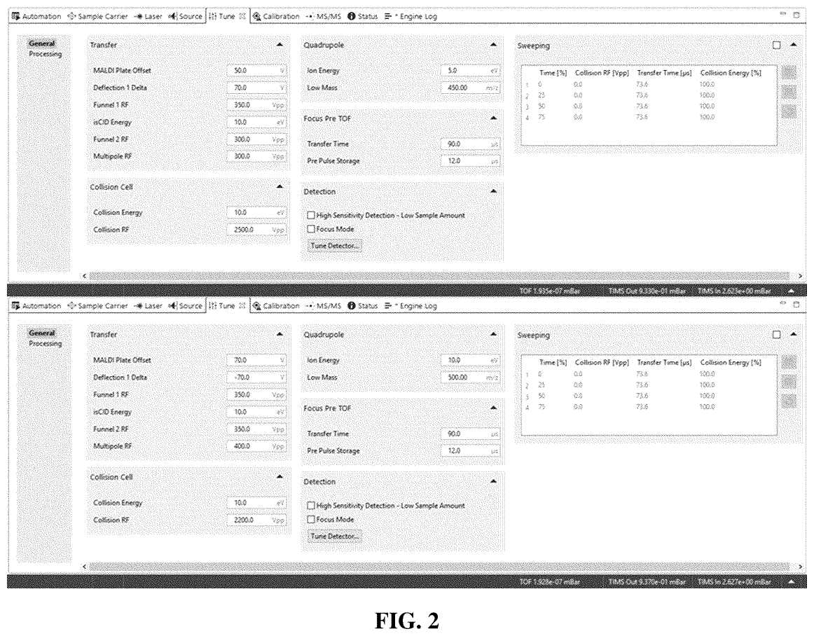

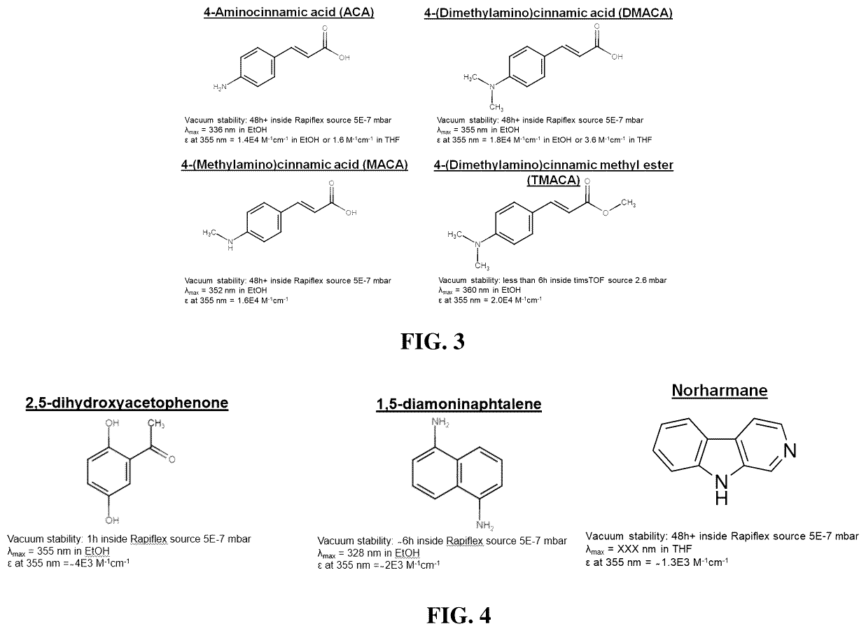

Profiling and MALDI IMS of thin tissue sections were performed on a timsTOF fleX mass spectrometer from Bruker Daltonics (Billerica, MA) in positive and negative ion modes (Spraggins et al., 2019). Total laser power (TLP) refers to the actual percent output of the MALDI laser when considering the values of the global attenuator offset, attenuator offset, attenuator range, and final attenuator value. The optimal laser power for each given matrices is defined as the maximum amount of laser power before oversampling occurs for a given pixel size. Data acquisition was performed using timsControl 3.1 and flexImaging 7.0 from Bruker Daltonics. Lipid identification was carried out comparing accurate mass (≤3.0 ppm or ≤2.0 ppm) with the LIPID MAPS database (Sud et al., 2007) and/or the Human Metabolome Database (HMDB) (Wishart et al., 2018) databases using in-house developed software along with exact mass matching with LC/MS data of tissue extract describe elsewhere (Esselman et al., 2025; Colley, 2023). Imaging data spatial resolution (i.e., image pixel size, or pitch) is defined by the step of the sample stage between pixels and was set to 5 μm or 10 μm and laser spot size was confirmed to be less than 5 μm or 10 μm, respectively (FIGS. 2A-2B), using either scanning electron microscopy (SEM) using a Zeiss Merlin (White Plains, NY) or brightfield microscopy with a Nikon Eclipse 90i microscope using a 40× objective (Nikon, Melville, NY). More detailed information regarding instrumental parameters can be found in FIG. 3.

### Data Analysis

Data analysis was performed with dataAnalysis 5.3 (Bruker Daltonics, Billerica, MA). MALDI IMS visualization was performed using SCiLS lab 2023c Pro. No data normalization was performed to allow for direct comparison of the raw intensity values (unless specifically stated otherwise in the figure caption).

MALDI IMS data were exported from the Bruker timsTOF file format (.d) into a custom binary format. Each pixel/frame contained centroid peaks spanning the entire acquisition range. These were reconstructed into a pseudo-profile mass spectrum using Bruker's SDK (v2.21). The data were m/z-aligned using at least six peaks commonly present in the majority of pixels, utilizing the msalign library (v0.2.0) (Monchamp et al, 2007; Migas, 2024). The mass axis of each dataset was calibrated using a minimum of four theoretical masses to achieve an accuracy of approximately ±1 ppm. Subsequently, the MALDI IMS data were normalized using a total ion current (TIC) approach, and an average mass spectrum was computed based on all pixels.

The average mass spectrum was normalized between 0 and 1, peak picked, deisotoped, and filtered using an automatically established signal-to-noise (S/N) threshold. IMS identification was performed using in-house developed software that associates detected peaks with tentative lipids from the LIPID MAPS Structure Database (LMSD) and MSDIAL (V69) database. Parameters for annotating peaks include [M+H]+, [M+Na]+, and [M+K]+ adducts in positive, [M−H]− and [M−CH3]− adducts in negative mode, and a search window of ±5 ppm. To provide more confident identification, IMS identifications were subsequently associated with LC-MS/MS identifications as well as reviewed against species reported in the literature.

### Example 2: Aminated Cinnamic Acid Matrices

### Characterization of Aminated Cinnamic Acid Derivatives as MALDI Matrices

Four MALDI matrices were screened for vacuum stability and extinction coefficient at 355 nm (FIG. 1). All but TMACA were found to be vacuum stable in the rapifleX source at 5.0E-7 mbar for at least 3 days. Vacuum stability was tested by measuring the mass of the slides coated in each matrix before and after storing them under vacuum for varying times (Table 6). For reference, DHA was also tested alongside as a commonly used dual polarity matrix. ACA, DMACA, and MACA were found to be vacuum stable for at least 48 h, even in a high vacuum ion source (˜2.0E-7 mbar). The minor variation observed for these vacuum stable candidates (0.1 mg) was within the expected error of the balance, which is ±0.1 mg. TMACA was visibly gone after just 3 hours inside the vacuum chamber, likely, though without being bound by theory, due to the increased volatility imparted by the esterification of the carboxylic acid function. Thus, TMACA was not tested in further matrix development experiments. DHA showed similar volatility to TMACA, largely subliming off after just a few hours.

With regard to extinction coefficient at 355 nm, all aminated cinnamic acid derivatives presented extremely high extinction coefficient at the typical MALDI wavelength of 355 nm in both ethanol or THF solution (1-2E4 M⁻¹ cm⁻¹, FIG. 1, Table 7) compared to conventional MALDI matrices (1-5E3 M⁻¹ cm⁻¹, FIG. 4, Table 7). The MALDI matrices disclosed herein were comparable in extinction coefficient values at 355 nm to CHDA ((E)-4-(2,5-dihydroxyphenyl)but-3-en-2-one) and HDMP ((E)-4-(4-hydroxy-3,5-dimethoxyphenyl)but-3-en-2-one), which are synthetically produced MALDI matrix candidates that have among the highest extinction coefficients at 355 nm (Yang et al., 2018). A high extinction coefficient, as demonstrated for the presently disclosed matrices, can allow for use of a substantially reduced laser power while maintaining efficient desorption and ionization during the MALDI process, which greatly reduces laser spot sizes at the sample surface, enabling higher spatial resolution analysis without oversampling.

TABLE 7
Optical constant measurements for various MALDI
matrices performed in tetrahydrofuran.

| | | ε at 355 nm |
|---|---|---|
| Matrix | Λₘₐₓ (nm) | (M⁻¹cm⁻¹) |
| DHA | 355 | ~4E3 |
| DAN | 328 | ~2E3 |
| Nor | 340 | ~1E3 |
| ACA | 340 | ~1E4 |
| MACA | 352 | ~1E4 |
| DMACA | 355 | ~2E4 |
| TMACA | 360 | ~1E4 |

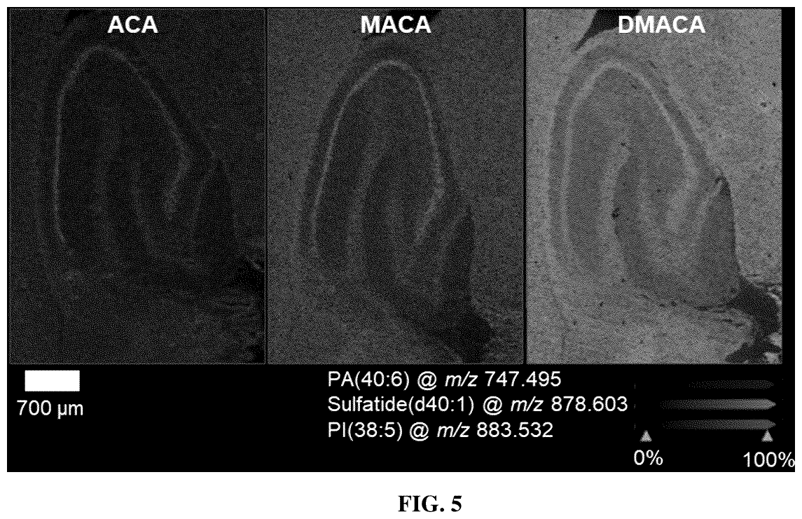

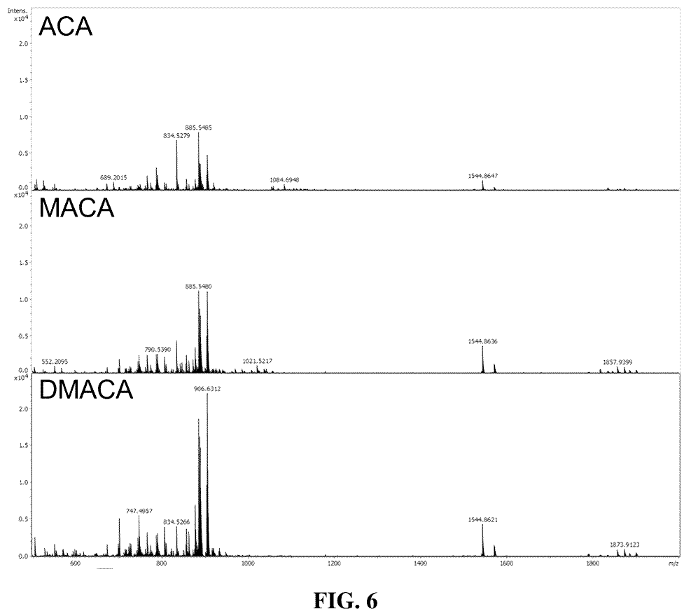

A negative mode lipid MALDI IMS experiment at 20 μm spatial resolution was performed on serial sagittal mouse brain tissue sections at the same laser power used for typical DAN MALDI IMS to evaluate which matrix from among DMACA, ACA, and MACA performed the best in the analysis of phospholipids. FIG. 5 shows ion images demonstrating that DMACA performed better for phospholipids vs ACA and MACA due to its higher extinction coefficient at 355 nm, which, without being bound by theory, allowed for more efficient desorption/ionization (FIG. 6). On the other hand, ACA demonstrated less in-source fragmentation of ganglioside species enabling the generation of a more accurate molecular profile than DMACA or MACA for thermally labile molecules (FIG. 7, FIG. 8). Higher order gangliosides are known to fragment easily, losing sialic acid residues, resulting in chemical structures that are the same as those of GM (mono-sialylated) gangliosides. As such, most MALDI experiments will show a higher abundance of GM species relative to GD (di-sialylated) and GT (tri-sialylated) lipids. However, compared to DMACA and MACA, ACA delivered a GM/GD ratio much closer to 1, a ratio known to be closer to reality in the murine brain (Ando, 1985). In this manner, DMACA was shown to exhibit favorable overall sensitivity and ACA was shown to minimize MALDI-induced ganglioside fragmentation.

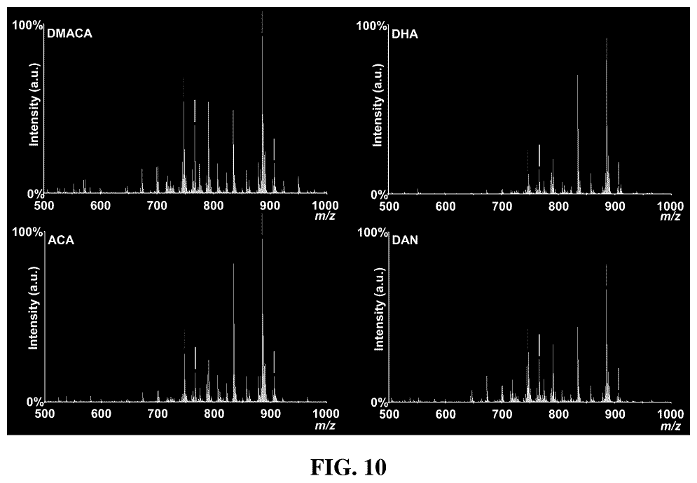

Image quality and sensitivity of DMACA and ACA were further compared to traditional lipid matrices DAN and DHA. Sagittal mouse brain tissue sections were imaged using 20% total laser power, typical for conventional MALDI matrices such as DHA and DAN on the Bruker timsTOF flex platform. Overlays of five ion images and the overall average spectra for IMS experiments using DMACA, ACA, DAN, and DHA are shown in FIG. 9 and FIG. 10. An H&E-stained image is provided to provide histological context. The ion images were visualized together and without any intensity normalization to provide a direct qualitative comparison of the raw intensity for each of the selected ions. The highest intensity ions can be observed in all cases. For example, PI(38:4) (m/z 885.549, red) is the base peak for all the matrices tested, and its distribution is apparent in all cases. However, it is evident that DMACA and ACA are much more sensitive for sphingomyelins (e.g., m/z 906.633, green), and DMACA is the most sensitive overall across all selected ions.

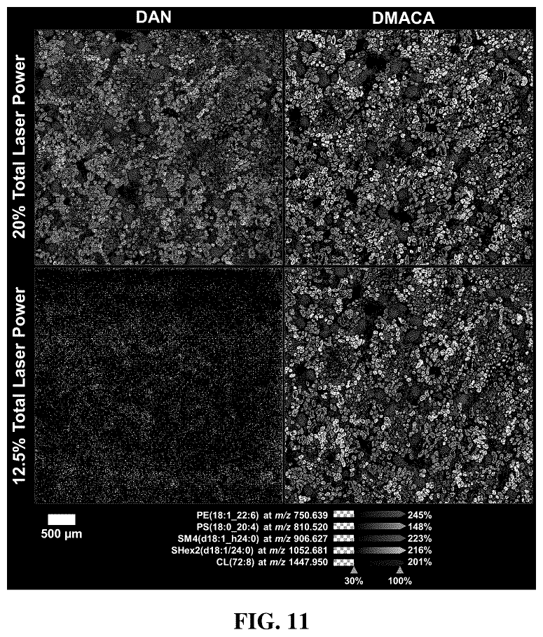

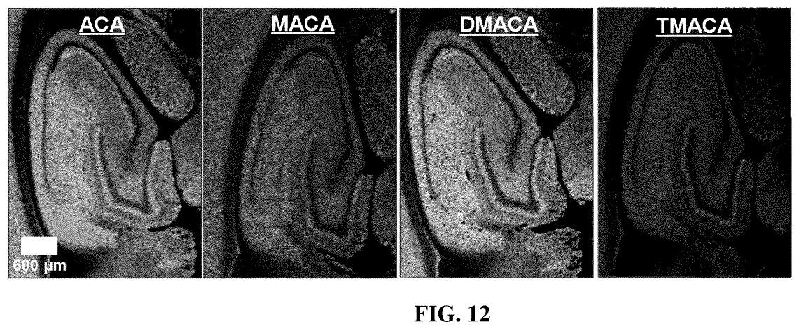

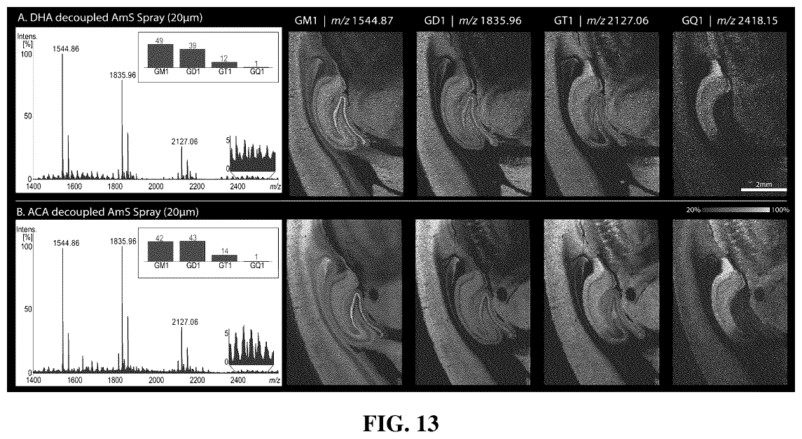

Further investigation into the differences between ACA, DMACA, DAN, and DHA for both phospholipids and gangliosides was carried out. For phospholipids, DMACA shows a nearly 2-3-fold increase in signal and 3-4-fold increase in signal-to-noise compared to DAN (FIG. 11). On the other hand, ACA presents a more accurate ganglioside lipids profile and a reduction in known in-source fragmentation of lipids compared to DHA (FIG. 12 and FIG. 13). During this assessment, both ACA and DMACA were found to form matrix/analyte adducts which were not identifiable in a search of an exact mass database. Subtraction of the neutral mass of the matrices from these unidentified signal values typically led to the proper identification of the exact mass of the adducted lipid molecule.

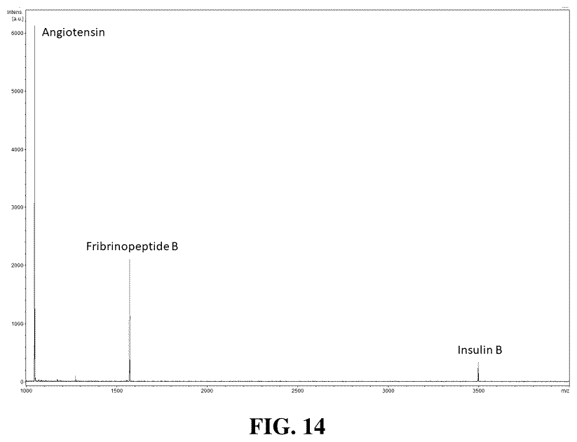

ACA and DMACA were then tested for use with peptides and protein analytes. Typical MALDI analysis of both classes of molecules involves the acidification of the matrix solution to allow for proper ionization. For both matrices, peptides and proteins from standards solution could be detected, but DMACA performed significantly better when compared to conventional MALDI matrices such as CHCA (FIGS. 14-15). Further evaluation of both ACA and DMACA showed that ACA degrades via a hydrolysis pathway in presence of acid, which is key to peptide and protein analysis (FIG. 16). This was clearly observed through a steep color change in the matrix solution from yellow to deep orange when TFA was added to the ACA solution.

ACA vs. DHA for Ganglioside Imaging

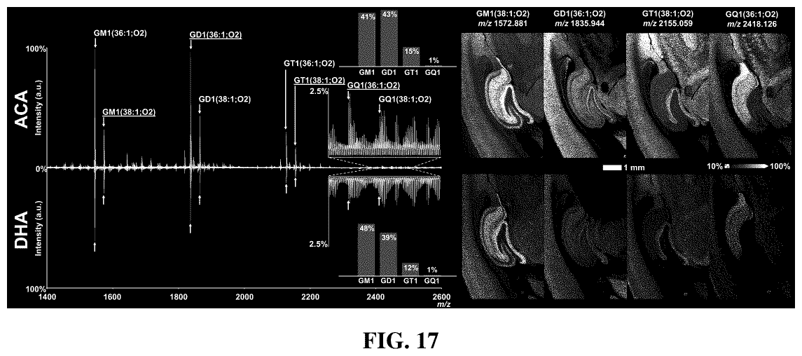

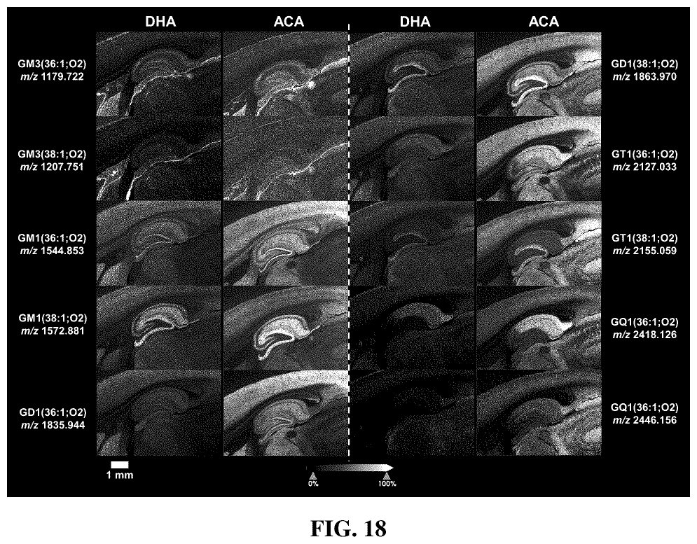

Gangliosides are a class of sialic acid-containing glycosphingolipids, which at high levels are primarily found in the central nervous system (CNS). They are typically anchored to the surface of the cell wall, where they are used in cell-to-cell communication by facilitating cell recognition, but they can also serve as receptors for protein interactions. IHC and MALDI IMS are the two predominant techniques used to study the spatial distribution of gangliosides. A MALDI IMS sample preparation method using DHA for gangliosides is known in the art and allows for improved visualization while minimizing in-source fragmentation of higher order gangliosides (Djambazova et al., 2023b; Djambazova et al., 2024). FIG. 17 shows a direct comparison of ACA and DHA using the previously described decoupled matrix spray method. When comparing the average spectra from the MALDI IMS experiments collected from the hippocampus region of mouse brain tissue, an increase in the intensity of di-sialylated (GD1) and tri-sialylated (GT1) gangliosides compared to the mono-sialylated species was observed, suggesting without being bound by theory a reduction in MALDI-induced fragmentation when using ACA. Detecting that GDs are more abundant than GMs with ACA appears more aligned with previously reported LC-MS data from murine brain and it is likely a better representation of the actual ratio of these molecules in biological systems (Ando et al., 1985). MALDI IMS experiments at 20 μm pixel sizes highlight the intensity disparity between ACA and DHA, where ACA consistently produces higher intensity signals for gangliosides than DHA, resulting in higher quality IMS images (FIG. 18). This can be seen in FIG. 17 where all gangliosides have higher intensity and image quality. This is especially the case for the highest order ganglioside detected, GQ1(36:1;O2) at m/z 2418.126, where the ion is only observed in the highest intensity regions of the tissue when using DHA, but ACA delivers a much more comprehensive and nuanced picture of its tissue distribution. In this manner, ACA is shown to better reveal ganglioside distributions in brain tissue and its vacuum stability. ACA offers a new alternative to conventional MALDI matrices for the spatial elucidation of this important class of molecules in both normal and diseased tissues.

### DMACA vs DAN for Dual Polarity MALDI IMS

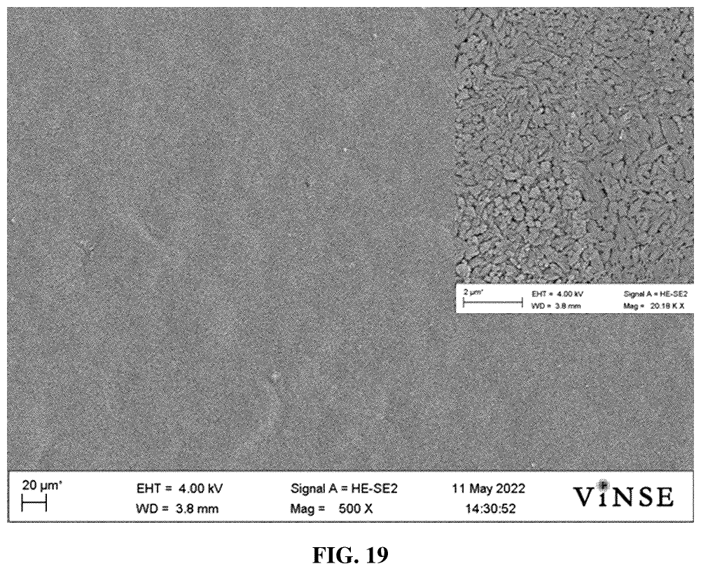

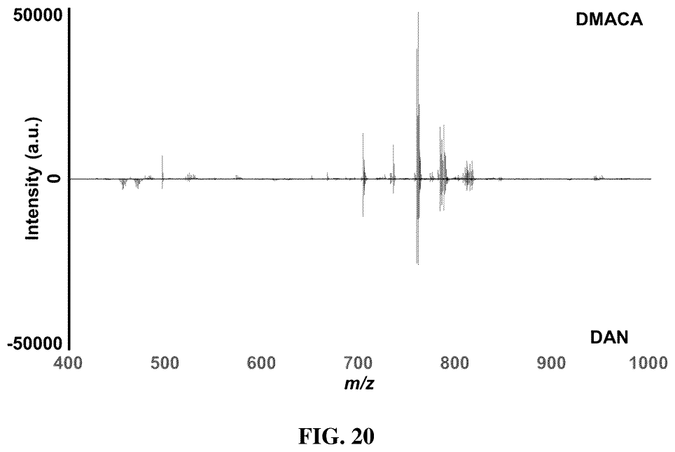

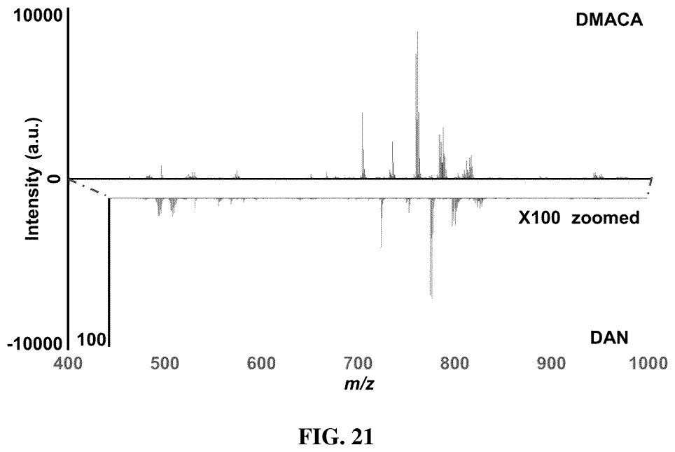

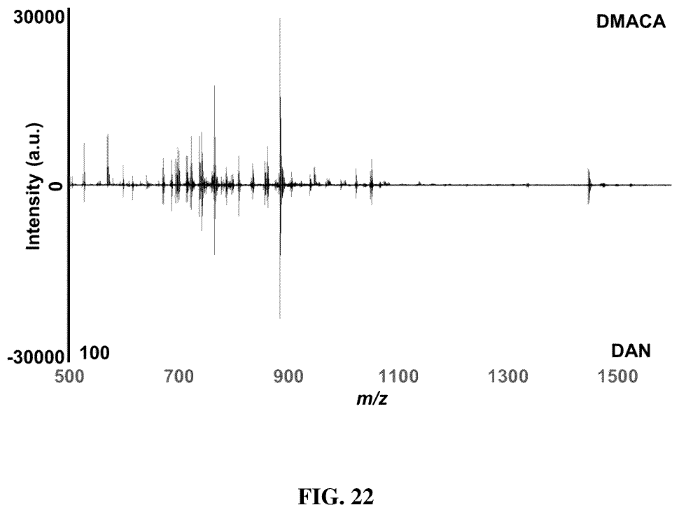

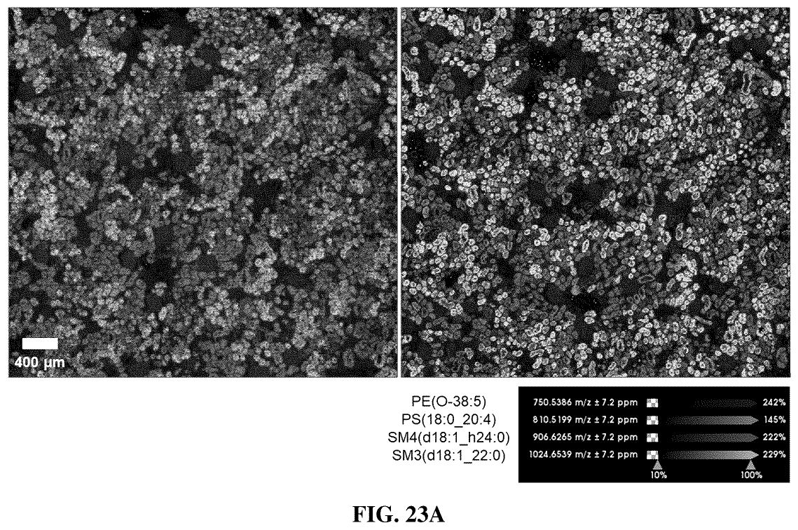

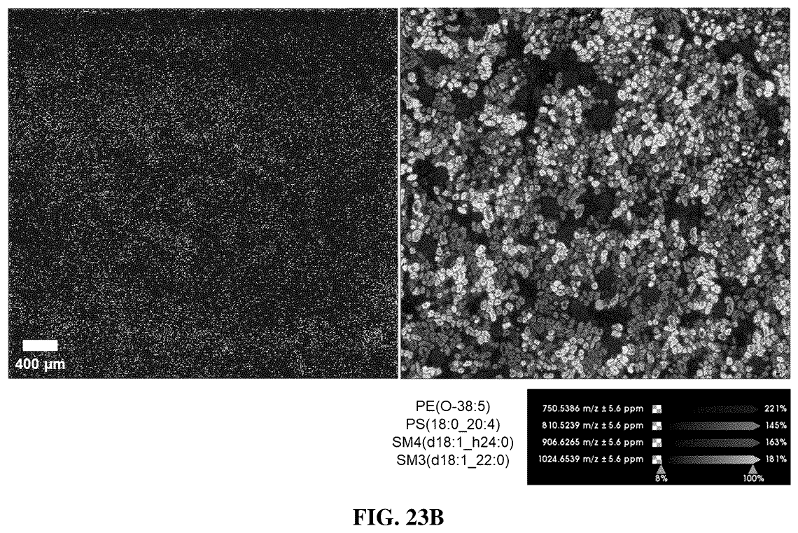

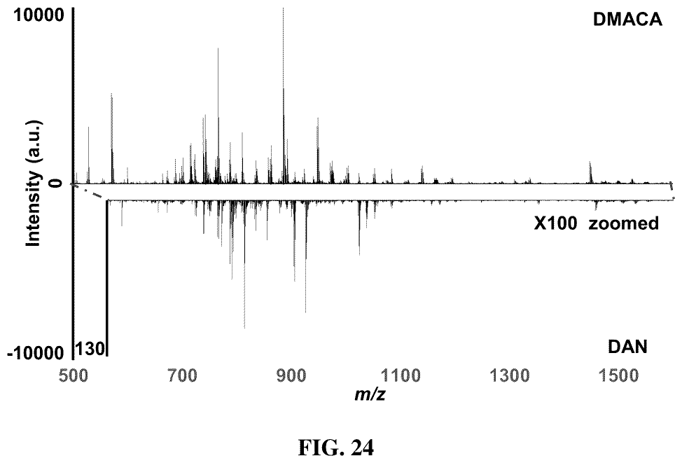

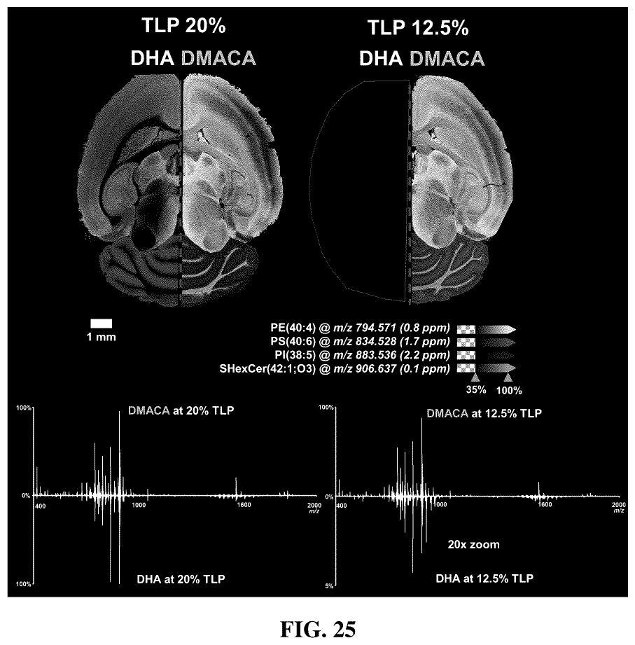

Due to the performance of DMACA in dual polarity analysis of phospholipids using MALDI IMS, further tests were conducted directly against DAN, a well-established and characterized dual polarity lipid matrix for samples of human kidney biopsies at 10 μm spatial resolution. (Thomas et al., 2012; Angerer et al., 2021). A water free spraying technique was used to generate a highly homogeneous matrix (DMACA) layer with sub-micron matrix crystals on a tissue. The average crystal size of the matrix layer was between 300-500 nm, as shown on FIG. 19. The DMACA matrix crystal layer represents a 2-fold decrease in the crystal size as compared to the DAN matrix crystal layer. FIG. 11A and FIG. 11B show a direct comparison between DMACA and DAN at a total laser power (TLP) of 20% (optimal for DAN analysis) and a TLP of 12.5% (optimal for DMACA) using a timsTOF flex MALDI IMS system. At 20% TLP both DAN and DMACA produced a similar amount of lipid signals, but for those signals known to be intact lipids DMACA generated nearly a 2-3-fold increase in ion signal without increasing noise. (FIG. 20, FIG. 21, FIG. 22) When using a TLP of 12.5%, DMACA still produces high-quality ion images, while DAN no longer produced ions above the limit of detection of the mass spectrometer, as seen in FIG. 23. This also led to a 3-fold reduction in in-source fragment for DMACA at 12.5% TLP compared to DAN at its optimal laser power. Reduction in laser power corresponded to a reduction in laser spot size on target from ˜5 to ˜4 μm, allowing accurate 5 μm MALDI IMS without over sampling using the timsTOF fleX. This dramatic reduction in laser energy concurrently reduces the laser beam diameter at the sample surface from ˜6 μm at 20% total laser power to ˜4.5 μm at 12.5% (FIG. 1B, FIG. 21, FIG. 24). Although sampling a smaller area results in a slight reduction of signal (˜2×), DMACA still produces data with an intensity similar to DAN at 20% total laser power at a lower energy and a significantly smaller sample ablation area. FIG. 25 shows a similar experiment comparing DMACA and DHA, where DMACA also shows improved performance for negative mode phospholipids.

### DMACA Sublimation

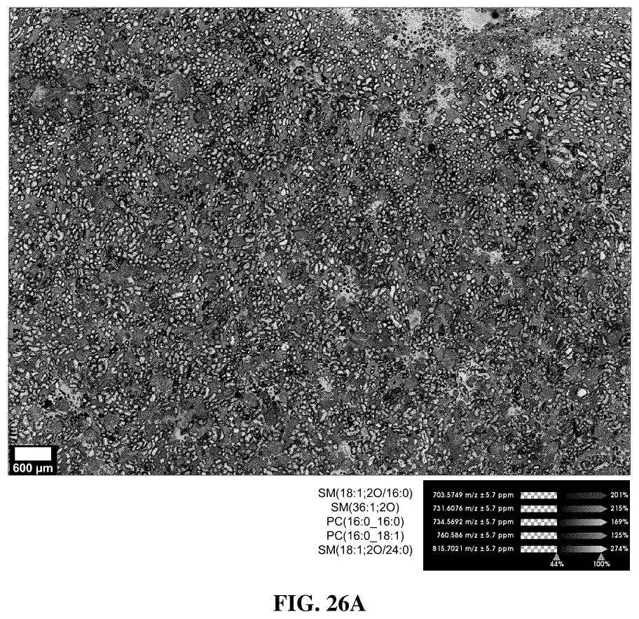

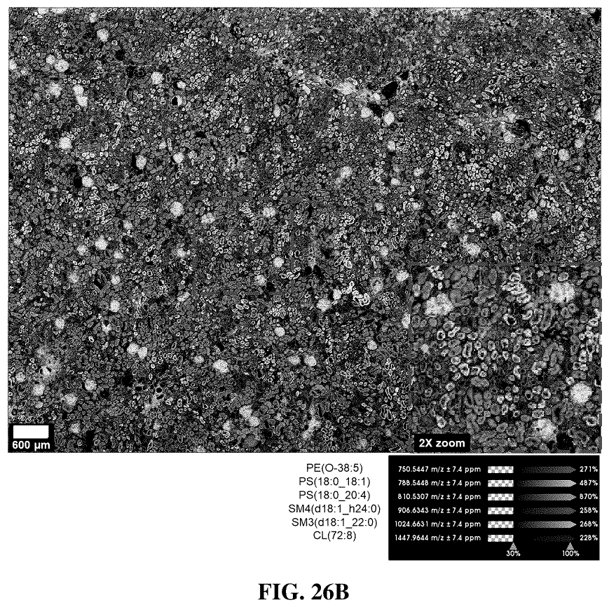

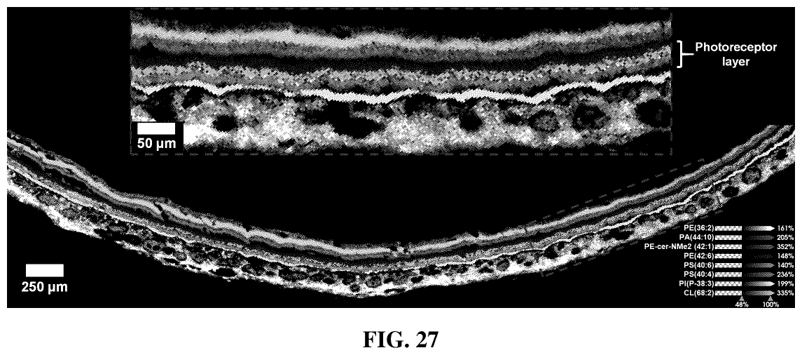

The DMACA was subjected to sublimation conditions to generate an on-tissue thin film over a human kidney biopsy at 10 μm spatial resolution and a human eye at 5 μm spatial resolution (FIG. 26 and FIG. 27). For the human kidney sample, 10 μm resolution of the inter tubule space along with intra-glomeruli heterogeneity m/z 750.545 in purple, tentatively identified as PE(O-38:5) and m/z 788.545 in teal, tentatively identified as PS(18:0_18:1) (FIG. 26) were clearly observed. As for the human eye, three different lipids were localized at subcellular level inside of the photoreceptor layer of the retina (PL) (FIG. 27). The PL is highlighted from bottom to top by m/z 795.494 in red, tentatively identified as PA(44:10), m/z 1403.991 in teal, tentatively identified as CL(68:2), and m/z 818.5679 in blue, tentatively identified as PE(42:6).

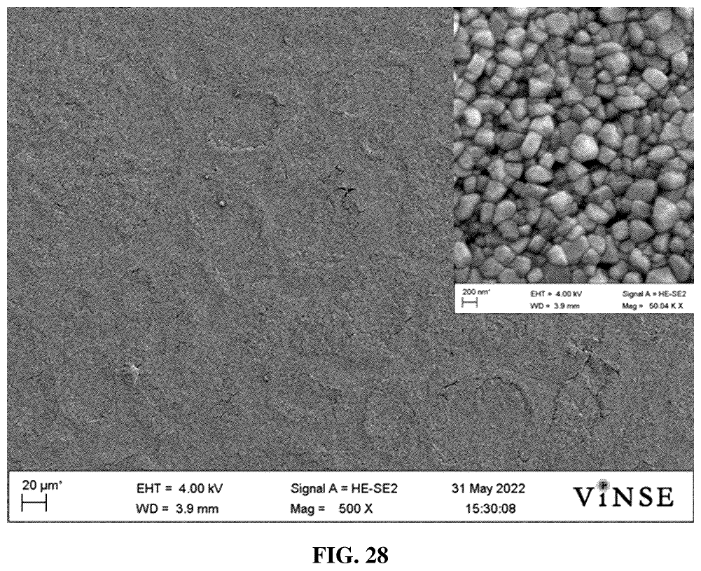

Further characterization of DMACA sublimated layers was performed using SEM and showed a highly homogenous layer of crystal with an average of 250 nm on tissue (FIG. 28). Without being bound by theory, the small crystal of the present matrices may help the desorption/ionization process by generating finer particles from the nanoscale crystals during desorption, thereby allowing for more ion to be produced in a similar fashion to what has been previously observed with other MS ion sources (Juraschek et al., 1999; Robichaud et al., 2014).

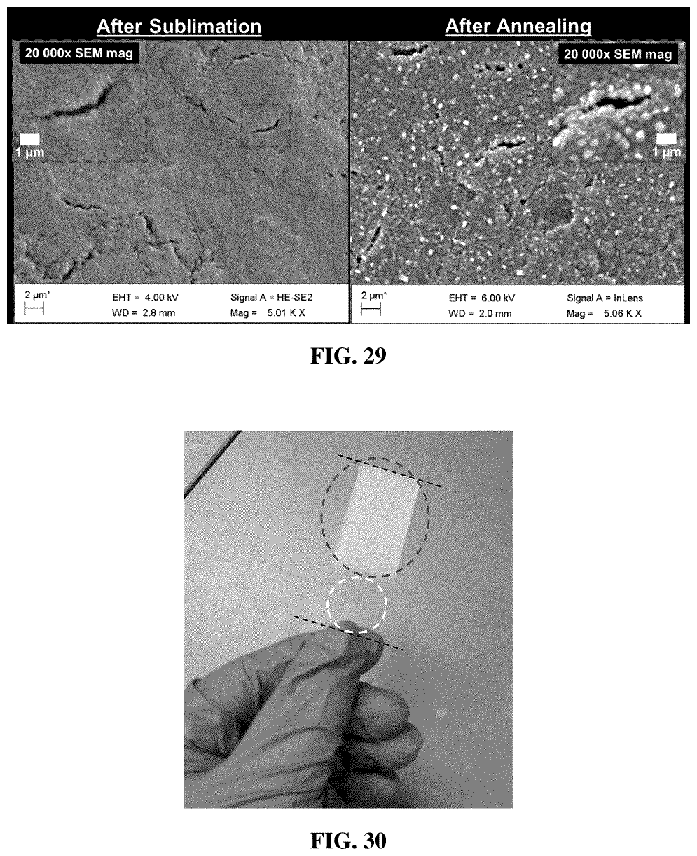

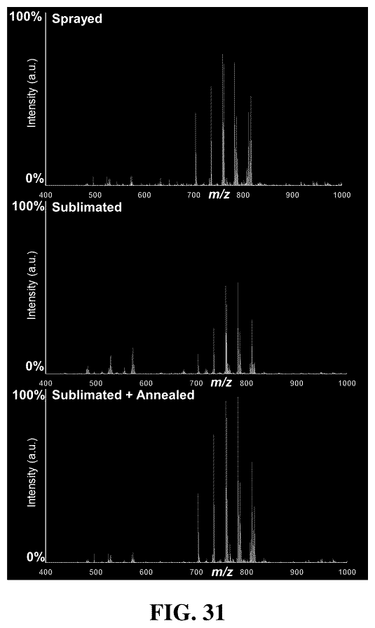

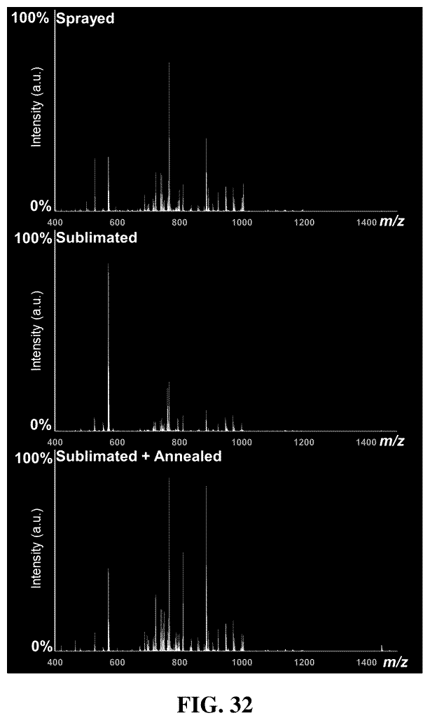

Further development of methods for sublimating MALDI matrices was pursued to determine the capability of cinnamic acid matrices deposited in the manner described herein to enable high resolution imaging. Towards that end, DMACA was subjected to various sublimation conditions (Thomas et al., 2012; Huizing et al., 2019; Hankin et al., 2007). ‘Thin’ sublimated layers of DMACA (˜0.22 μg/mm2) produced a semi-transparent, iridescent matrix film that led to low ion signals compared to thicker layers (0.5-1.0 μg/mm2), which are more typical for MALDI IMS experiments. When analyzed by scanning electron microscopy, the resulting sublimated thin layer showed no visible crystals on the tissue (FIG. 29). This observation suggested, without being bound by theory, that when applied by sublimation, the matrix is amorphous, which indicated that the matrix sublimation method might benefit from re-crystallization. Re-crystallization in MALDI IMS workflows is often performed using solvents through a custom made vapor chamber using a variety of solvent compositions (Yang & Caprioli, 2011; Bouschen et al., 2010) However, solvent-based recrystallization methods leads to analyte delocalization in most cases. To overcome this challenge, the inventors developed a simple solvent-free approach using only heat to induce crystallization (i.e., heat annealing) of the amorphous matrix layer. FIG. 29 shows scanning electron microscopy images of the matrix layer before and after heat annealing. The heat annealing method was performed by placing the matrix-coated slide on a hot plate (100° C.) for 30 s. Heat annealing led to a clear change in the coloration, a conversion from a transparent to opaque thin matrix layer, indicating a change to the optical properties of the surface (FIG. 30). Scanning electron microscopy images show this change in the transition from a crystal-free layer to a crystalline layer with crystal sizes ranging from 200 to 500 nm after heat annealing (FIG. 29). This process was also tested with conventional MALDI matrices known in the art, such as DHA and DAN, but was found to not work due to the volatility of these matrices. Both matrices de-sublimated from the slide at atmospheric pressure either completely (DHA) or partially (DAN) in the fume hood while attempting heat annealing. This heat annealing process increases ion signals by roughly 2-to-3-fold compared to methods without re-crystallization (FIG. 31, FIG. 32). Moreover, sublimation of DMACA followed by annealing was also found to be more sensitive than matrix spraying methods without the risk of delocalization for high spatial resolution experiments (FIG. 31, FIG. 32). Both observations are tissue type, molecular class, and matrix dependent.

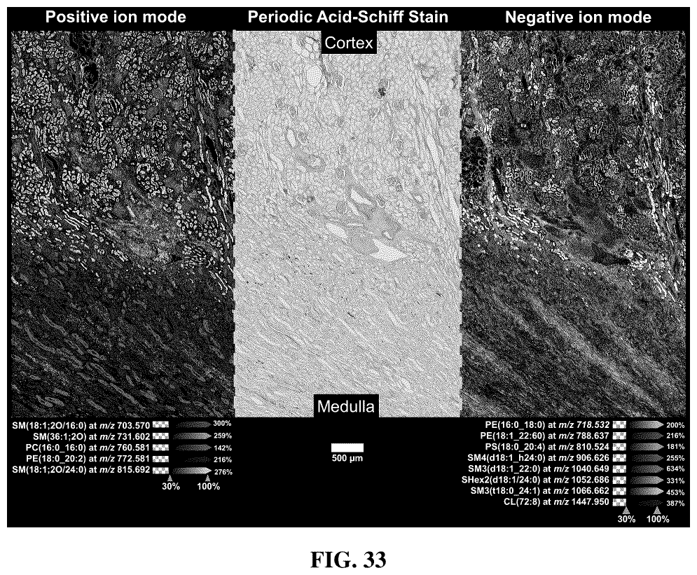

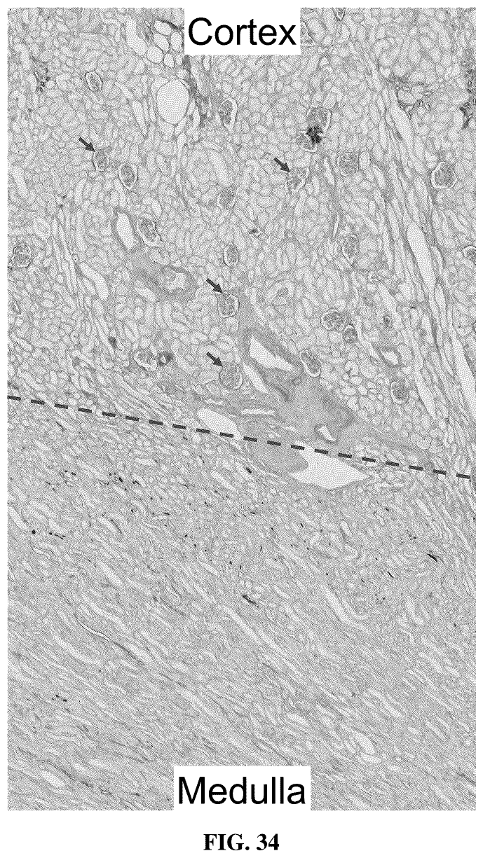

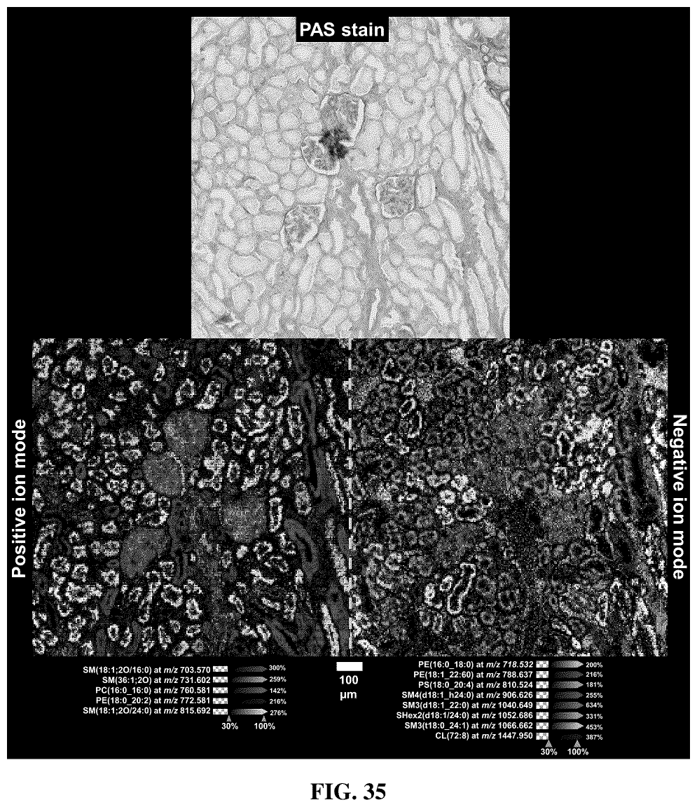

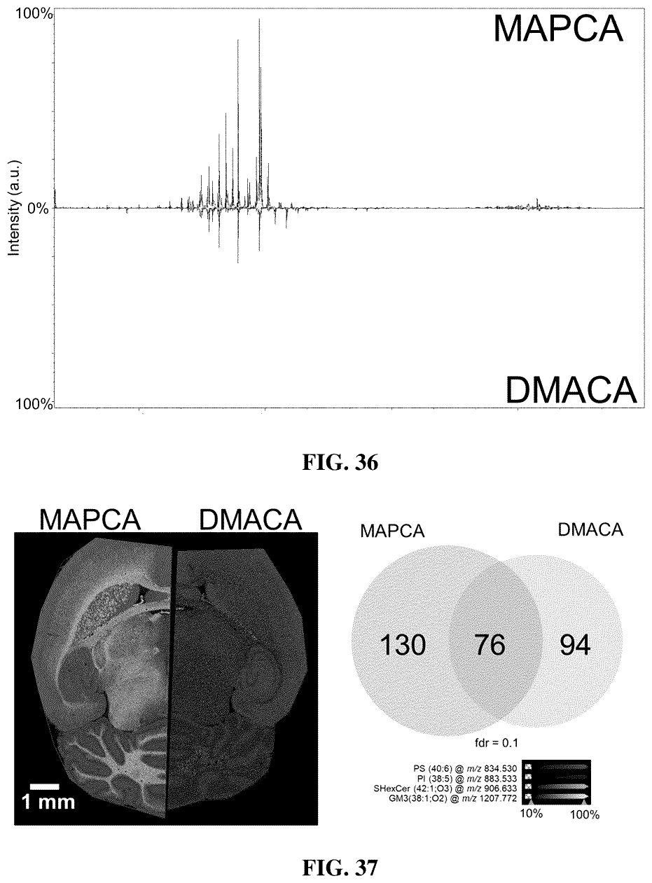

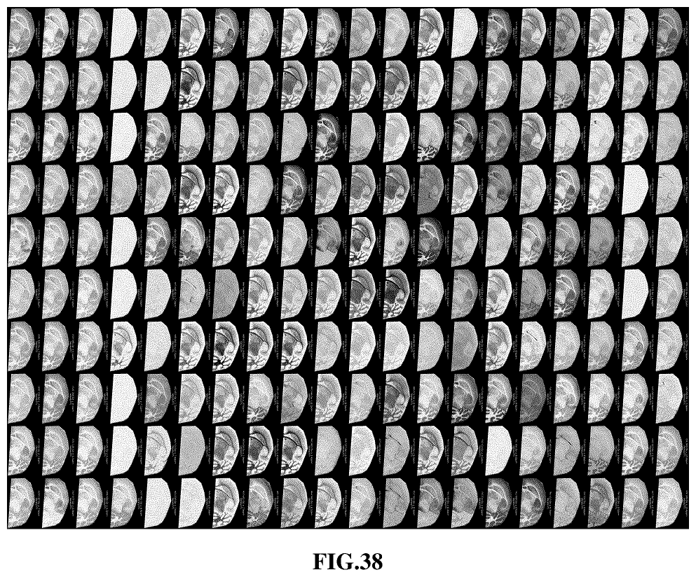

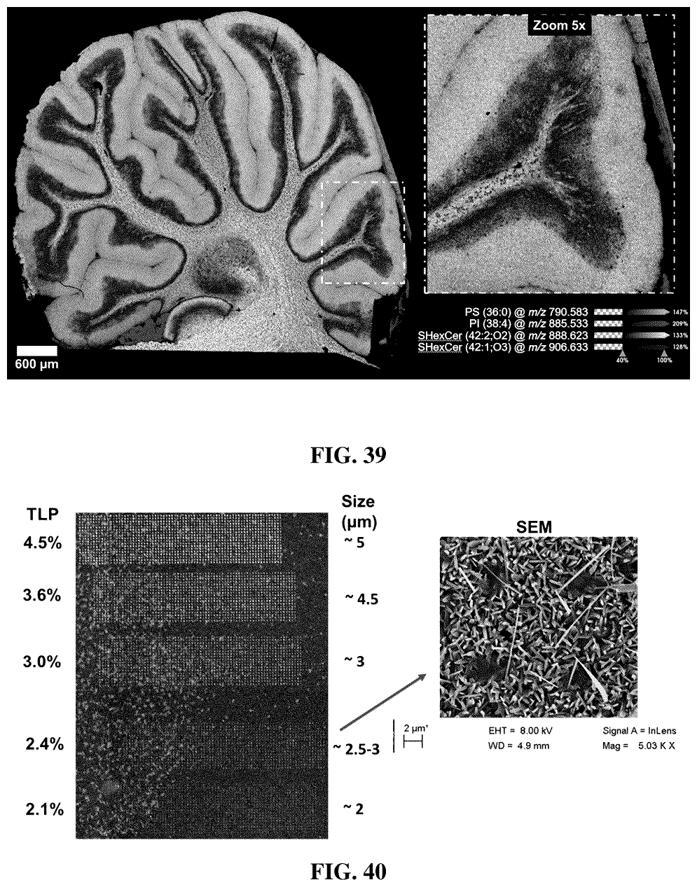

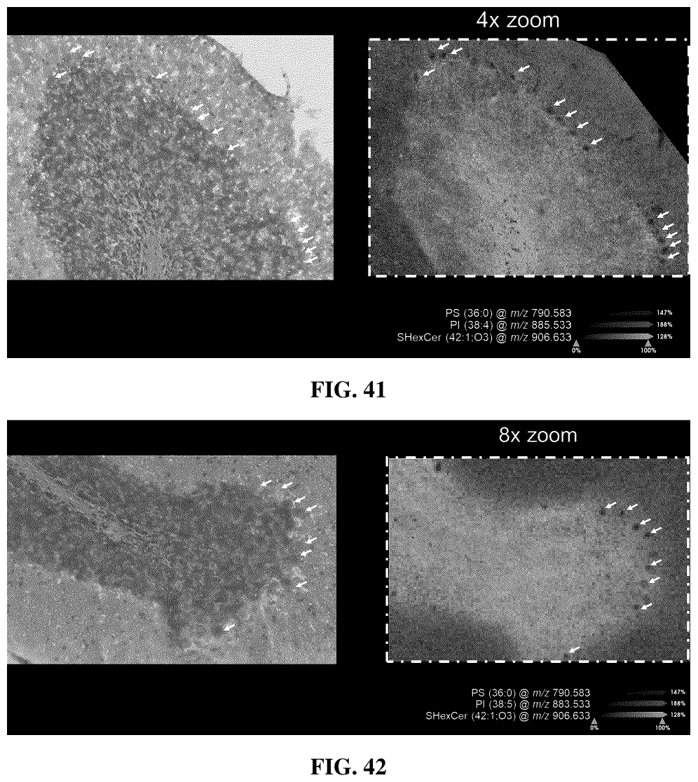

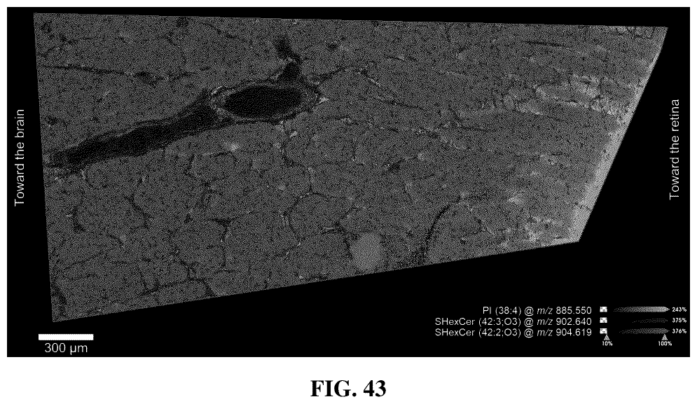

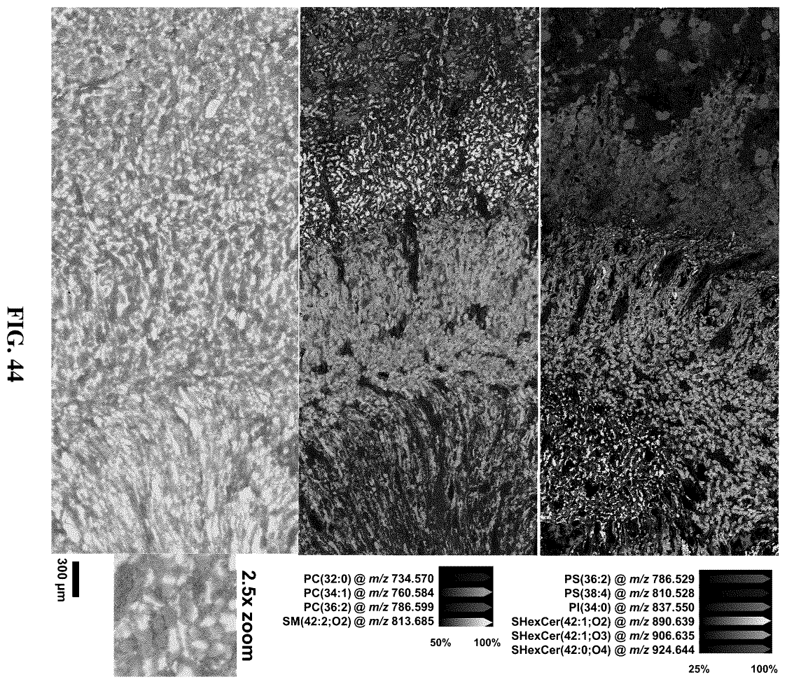

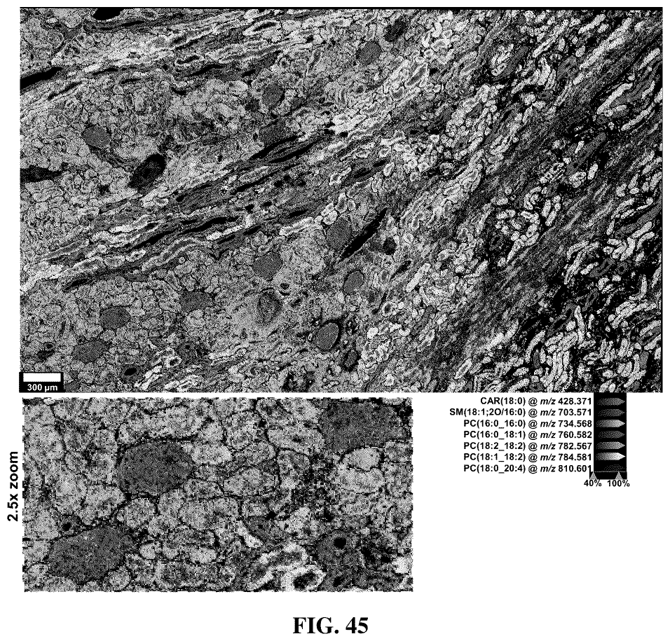

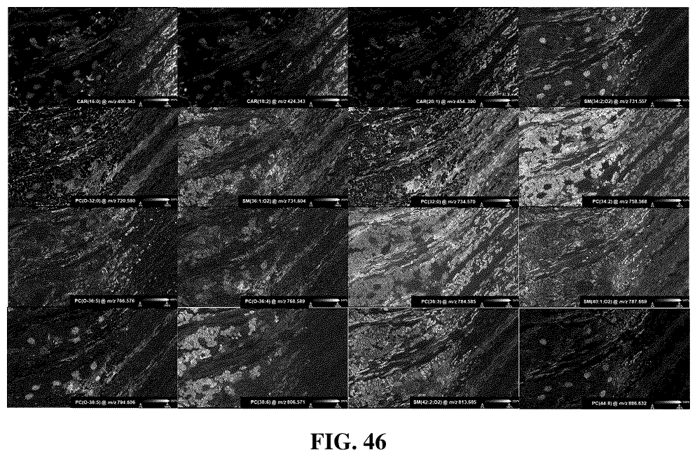

The capability for mapping lipids at high spatial resolution was demonstrated by collecting MALDI IMS data from a fresh-frozen human kidney tissue section using 5 μm pixels, which is presently the practical limit for most commercial mass spectrometers. FIG. 33 shows selected ion images for both positive (5 lipid species) and negative (8 lipid species) ion modes. A periodic acid-Schiff (PAS)-stained image (stained after MALDI acquisition) is included for reference to highlight the histology of the tissue (FIG. 34). For both polarities, ions reported as red represent glomeruli, while green-reported ion species delineate proximal tubules, which are only found in the cortex region of the kidney. Lipids associated with the proximal tubules and descending limb are colored in orange, while those colored purple were observed in the ascending limb of the nephron. These span from the cortex to the medulla of the kidney. It is noted that these assignments to specific components of the nephron are made possible by the high-quality PAS-stained images that can be acquired post-MALDI IMS-acquisition using the methods disclosed herein. In addition to enabling smaller IMS pixel sizes, the lower laser energy also induces less tissue damage during irradiation, improving performance for subsequent microscopy modalities as part of multimodal studies (Esselman et al., 2023; Esselman et al., 2024). Along with these selected ion signals, ˜300 lipids were annotated, of which ˜120 lipids were found in positive ion mode and ˜180 in negative ion mode at 5 μm pixel size. While the number of total ions detected is lower than typically observed at 10 μm pixel size in human kidney (˜400 total lipids), these data showcase the advantages of DMACA for high spatial resolution MALDI IMS. The performance of this method allows for molecular heterogeneity to be observed, even within individual components of the nephron, including individual glomeruli (FIG. 35). In this manner, DMACA is shown to be highly sensitive, and the matrix application methods disclosed herein are shown to provide the sensitivity necessary to produce ion images at cellular resolution without the need for custom instrumental setups.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Caprioli, R. M., Farmer, T. B. & Gile, J. Molecular imaging of biological samples: Localization of peptides and proteins using MALDI-TOF MS. Analytical Chemistry 69, 4751-4760, doi:10.1021/ac970888i (1997).
Stoeckli, M., Chaurand, P., Hallahan, D. E. & Caprioli, R. M. Imaging mass spectrometry: A new technology for the analysis of protein expression in mammalian tissues. Nat. Med. 7, 493-496, doi:10.1038/86573 (2001).
Grove, K. J., Frappier, S. L. & Caprioli, R. M. Matrix Pre-Coated MALDI MS Targets for Small Molecule Imaging in Tissues. Journal of the American Society for Mass Spectrometry 22, 192-195, doi:10.1007/s13361-010-0013-8 (2011).
Yang, J. & Caprioli, R. M. Matrix Sublimation/Recrystallization for Imaging Proteins by Mass Spectrometry at High Spatial Resolution. Analytical Chemistry 83, 5728-5734, doi:10.1021/ac200998a (2011).
Angel, P. M., Spraggins, J. M., Baldwin, H. S. & Caprioli, R. Enhanced Sensitivity for High Spatial Resolution Lipid Analysis by Negative Ion Mode Matrix Assisted Laser Desorption Ionization Imaging Mass Spectrometry. Analytical Chemistry 84, 1557-1564, doi:10.1021/ac202383m (2012).
Porta Siegel, T. et al. Mass Spectrometry Imaging and Integration with Other Imaging Modalities for Greater Molecular Understanding of Biological Tissues. Molecular Imaging and Biology 20, 888-901, doi:10.1007/s11307-018-1267-y (2018).
Zavalin, A. et al. Direct imaging of single cells and tissue at sub-cellular spatial resolution using transmission geometry MALDI MS. Journal of Mass Spectrometry 47, 1473-1481, doi:10.1002/jms.3108 (2012).
Niehaus, M., Soltwisch, J., Belov, M. E. & Dreisewerd, K. Transmission-mode MALDI-2 mass spectrometry imaging of cells and tissues at subcellular resolution. Nature Methods 16, 925-931, doi:10.1038/s41592-019-0536-2 (2019).
Yin, R., Burnum-Johnson, K. E., Sun, X., Dey, S. K. & Laskin, J. High spatial resolution imaging of biological tissues using nanospray desorption electrospray ionization mass spectrometry. Nature Protocols 14, 3445-3470, doi:10.1038/s41596-019-0237-4 (2019).
Unsihuay, D. et al. High-resolution imaging and identification of biomolecules using Nano-DESI coupled to ion mobility spectrometry. Analytica Chimica Acta 1186, 339085, doi:https://doi.org/10.1016/j.aca.2021.339085 (2021).
Nazari, M. & Muddiman, D. C. Cellular-level mass spectrometry imaging using infrared matrix-assisted laser desorption electrospray ionization (IR-MALDESI) by oversampling. Analytical and Bioanalytical Chemistry 407, 2265-2271, doi:10.1007/s00216-014-8376-5 (2015).
Wang, X. et al. Hydroxyflavones as a New Family of Matrices for MALDI Tissue Imaging. Analytical Chemistry 85, 7566-7573, doi:10.1021/ac401595a (2013).
Shirey, K. A. et al. The TLR4 antagonist Eritoran protects mice from lethal influenza infection. Nature 497, 498-502, doi:10.1038/nature12118 (2013).
Scott, A. J. et al. Mass spectrometry imaging enriches biomarker discovery approaches with candidate mapping. Health physics 106, 120-128, doi:10.1097/HP.0b013e3182a4ec2f (2014).
Scott, A. J. et al. Norharmane Matrix Enhances Detection of Endotoxin by MALDI-MS for Simultaneous Profiling of Pathogen, Host, and Vector Systems. Pathogens and disease 74, doi:10.1093/femspd/ftw097 (2016).
Huang, P. et al. Toward the Rational Design of Universal Dual Polarity Matrix for MALDI Mass Spectrometry. Analytical Chemistry 92, 7139-7145, doi:10.1021/acs.analchem.0c00570 (2020).
Yang, J., Norris, J. L. & Caprioli, R. Novel vacuum stable ketone-based matrices for high spatial resolution MALDI imaging mass spectrometry. Journal of Mass Spectrometry 53, 1005-1012, doi:10.1002/jms.4277 (2018).
Hankin, J. A., Barkley, R. M. & Murphy, R. C. Sublimation as a method of matrix application for mass spectrometric imaging. Journal of the American Society for Mass Spectrometry 18, 1646-1652, doi:10.1016/j.jasms.2007.06.010 (2007).
Sud, M. et al. LMSD: LIPID MAPS structure database. Nucleic Acids Res 35, D527-D532, doi:10.1093/nar/gkl838 (2007).
Wishart, D. S. et al. HMDB 4.0: the human metabolome database for 2018. Nucleic Acids Res 46, D608-D617, doi:10.1093/nar/gkx1089 (2018).
Thomas, A., Charbonneau, J. L., Fournaise, E. & Chaurand, P. Sublimation of New Matrix Candidates for High Spatial Resolution Imaging Mass Spectrometry of Lipids: Enhanced Information in Both Positive and Negative Polarities after 1,5-Diaminonapthalene Deposition. Analytical Chemistry 84, 2048-2054, doi:10.1021/ac2033547 (2012).
Angerer, T. B., Bour, J., Biagi, J.-L., Moskovets, E. & Frache, G. Evaluation of 6 MALDI-Matrices for 10 m lipid imaging and on-tissue MSn with AP-MALDI-Orbitrap. bioRxiv, 2021.2010.2027.466111, doi:10.1101/2021.10.27.466111 (2021).
Juraschek, R., Dülcks, T. & Karas, M. Nanoelectrospray-more than just a minimized-flow electrospray ionization source. Journal of the American Society for Mass Spectrometry 10, 300-308, doi:https://doi.org/10.1016/S1044-0305(98)00157-3 (1999).
Robichaud, G., Barry, J. A. & Muddiman, D. C. IR-MALDESI mass spectrometry imaging of biological tissue sections using ice as a matrix. J Am Soc Mass Spectrom 25, 319-328, doi:10.1007/s13361-013-0787-6 (2014).
Zhou, Q., Fülöp, A. & Hopf, C. Recent developments of novel matrices and on-tissue chemical derivatization reagents for MALDI-MSI. Analytical and Bioanalytical Chemistry 413, 2599-2617 (2021).
Colley, M. E., Esselman, A. B., Scott, C. F. & Spraggins, J. M. High-Specificity Imaging Mass Spectrometry. Annual Review of Analytical Chemistry 17, 1-24 (2024).
Yang, M. et al. Nano-DESI Mass Spectrometry Imaging of Proteoforms in Biological Tissues with High Spatial Resolution. Analytical Chemistry 95, 5214-5222 (2023).
Ma, S. et al. High spatial resolution mass spectrometry imaging for spatial metabolomics: Advances, challenges, and future perspectives. TrAC Trends in Analytical Chemistry 159, 116902 (2023).
Djambazova, K. V., Van Ardenne, J. M. & Spraggins, J. M. Advances in imaging mass spectrometry for biomedical and clinical research. TrAC Trends in Analytical Chemistry 169, 117344 (2023).
Snyder, M. P. et al. The human body at cellular resolution: the NIH Human Biomolecular Atlas Program. Nature 574, 187-192 (2019).
Jain, S. et al. Advances and prospects for the Human BioMolecular Atlas Program (HuBMAP). Nature Cell Biology 25, 1089-1100 (2023).
Dufresne, M., Kruse, A. R. S., Allen, J., Gutierrez, D. & Spraggins, J. M. Deposition of matrix using an M5™ sprayer for high resolution MALDI analysis V.3, (2023).
Anderson, D. et al. Matrix Sublimation via In-House Developed Sublimation Apparatus, (2023).
Spraggins, J. M. et al. High-Performance Molecular Imaging with MALDI Trapped Ion-Mobility Time-of-Flight (timsTOF) Mass Spectrometry. Analytical Chemistry 91, 14552-14560 (2019).
Esselman, A. B. et al. In situ molecular profiles of glomerular cells by integrated imaging mass spectrometry and multiplexed immunofluorescence microscopy. Kidney International 107, 332-337 (2025).
Colley, M. E. et al. Bulk Untargeted LC-MS/MS Lipidomics, (2023).
Monchamp, P., Andrade-Cetto, L., Zhang, J. Y. & Henson, R. Signal processing methods for mass spectrometry. Systems Bioinformatics: An Engineering Case-Based Approach, Artech House Publishers (2007).
Migas, L. G. Spectral alignment based on MATLAB's ‘msalign’ function, (2024)
Ando, S. in Selected Topics from Neurochemistry (ed Neville N. Osborne) 439-486 (Pergamon, 1985).
Djambazova, K. V. et al. MALDI TIMS IMS of Disialoganglioside Isomers-GD1a and GD1b in Murine Brain Tissue. Analytical Chemistry 95, 1176-1183 (2023).
Djambazova, K. V. et al. MALDI TIMS IMS Reveals Ganglioside Molecular Diversity within Murine S. aureus Kidney Tissue Abscesses. Journal of the American Society for Mass Spectrometry 35, 1692-1701 (2024).
Huizing, L. R. S. et al. Development and evaluation of matrix application techniques for high throughput mass spectrometry imaging of tissues in the clinic. Clin Mass Spectrom 12, 7-15 (2019).
Bouschen, W., Schulz, O., Eikel, D. & Spengler, B. Matrix vapor deposition/recrystallization and dedicated spray preparation for high-resolution scanning microprobe matrix-assisted laser desorption/ionization imaging mass spectrometry (SMALDI-MS) of tissue and single cells. Rapid Communications in Mass Spectrometry 24, 355-364 (2010).
Esselman, A. B. et al. Microscopy-Directed Imaging Mass Spectrometry for Rapid High Spatial Resolution Molecular Imaging of Glomeruli. Journal of the American Society for Mass Spectrometry 34, 1305-1314 (2023).
Esselman, A. B. et al. A Streamlined Workflow for Microscopy-Driven MALDI Imaging Mass Spectrometry Data Collection. Journal of the American Society for Mass Spectrometry 35, 2795-2800, (2024).

1. A method of detecting an analyte comprising:
a) obtaining a sample comprising an analyte;
b) obtaining a matrix compound of the formula: 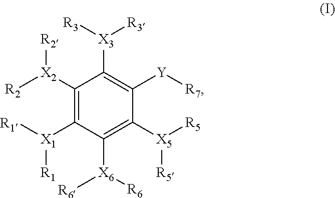
wherein:
X₁, X₂, X₃, X₅, and X₆ are each independently hydrogen, —C(O)—, or a proton-accepting atom, provided that if X₁ is hydrogen, R₁ and R_{1′} are absent; if X₃ is hydrogen, R₃ and R_{3′} are absent; and if X₅ is hydrogen, R₅ and R_{5′} are absent; and provided that at least one of X₁, X₂, X₃, X₅, and X₆ is not hydrogen;
R₁, R_{1′}, R₂, R_{2′}, R₃, R_{3′}, R₅, R_{5′}, R₆, and R_{6′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
Y is alkanediyl_{(C≤8)}, substituted alkanediyl_{(C≤8)}, alkenediyl_{(C≤8)}, substituted alkenediyl_{(C≤8)}, alkynediyl_{(C≤8)}, or substituted alkynediyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
—C(O)R₈, wherein:
R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof;
c) depositing the sample and the matrix compound on a surface to form an analyte-matrix co-crystal composition;
d) irradiating the analyte-matrix co-crystal composition with a laser;
e) detecting the analyte by mass spectrometry.

2. The method of claim 1, wherein the matrix compound is further defined as: 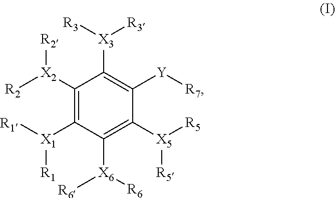
wherein:
X₁, X₂, X₃, X₅, and X₆ are each independently hydrogen, —C(O)—, or a proton-accepting atom, provided that if X₁ is hydrogen, R₁ and R_{1′} are absent; if X₃ is hydrogen, R₃ and R_{3′} are absent; and if X₅ is hydrogen, R₅ and R_{5′} are absent; and provided that at least one of X₁, X₂, X₃, X₅, and X₆ is not hydrogen;
R₁, R_{1′}, R₂, R_{2′}, R₃, R_{3′}, R₅, R_{5′}, R₆, and R_{6′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
Y is alkanediyl_{(C≤8)}, polar-substituted alkanediyl_{(C≤8)}, alkenediyl_{(C≤8)}, polar-substituted alkenediyl_{(C≤8)}, alkynediyl_{(C≤8)}, or polar-substituted alkynediyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
—C(O)R₈, wherein:
R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof;

3. The method of claim 1, wherein the matrix compound is further defined as: 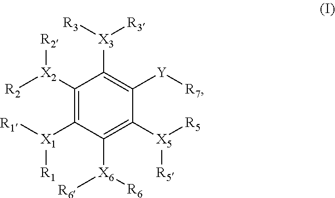
wherein:
X₁, X₂, X₃, X₅, and X₆ are each independently hydrogen, nitrogen, oxygen, sulfur, —C(O)—, or phosphorus provided that if X₁ is hydrogen, R₁ and R_{1′} are absent; if X₃ is hydrogen, R₃ and R_{3′} are absent; and if X₅ is hydrogen, R₅ and R_{5′} are absent; and provided that at least one of X₁, X₂, X₃, X₅, and X₆ is not hydrogen;
R₁, R_{1′}, R₂, R_{2′}, R₃, R_{3′}, R₅, R_{5′}, R₆, and R_{6′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
Y is alkanediyl_{(C≤8)}, polar-substituted alkanediyl_{(C≤8)}, alkenediyl_{(C≤8)}, polar-substituted alkenediyl_{(C≤8)}, alkynediyl_{(C≤8)}, or polar-substituted alkynediyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
—C(O)R₈, wherein:
R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof.

4. The method of claim 1, wherein the matrix compound is further defined as: 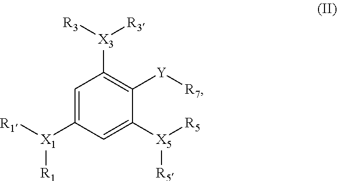
wherein:
X₁, X₃, and X₅ are each independently hydrogen, —C(O)—, or a proton-accepting atom, provided that if X₁ is hydrogen, R₁ and R_{1′} are absent; if X₃ is hydrogen, R₃ and R_{3′} are absent; and if X₅ is hydrogen, R₅ and R_{5′} are absent; and provided that at least one of X₁, X₃, and X₅ is not hydrogen;
R₁, R_{1′}, R₃, R_{3′}, R₅, and R_{5′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
Y is alkanediyl_{(C≤8)}, substituted alkanediyl_{(C≤8)}, alkenediyl_{(C≤8)}, substituted alkenediyl_{(C≤8)}, alkynediyl_{(C≤8)}, or substituted alkynediyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
—C(O)R₈, wherein:
R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof.

5. The method of claim 1, wherein the matrix compound is further defined as: 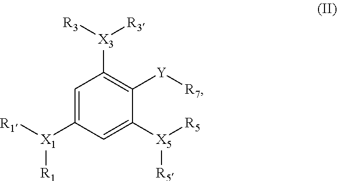
wherein:
X₁, X₃, and X₅ are each independently hydrogen, nitrogen, oxygen, sulfur, —C(O)—, or phosphorus provided that if X₁ is hydrogen, R₁ and R_{1′} are absent; if X₃ is hydrogen, R₃ and R_{3′} are absent; and if X₅ is hydrogen, R₅ and R_{5′} are absent; and provided that at least one of X₁, X₃, and X₅ is not hydrogen;
R₁, R_{1′}, R₃, R_{3′}, R₅, and R_{5′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
Y is alkanediyl_{(C≤8)}, polar-substituted alkanediyl_{(C≤8)}, alkenediyl_{(C≤8)}, polar-substituted alkenediyl_{(C≤8)}, alkynediyl_{(C≤8)}, or polar-substituted alkynediyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
—C(O)R₈, wherein:
R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof.

6. The method of claim 1, wherein the matrix compound is further defined as: 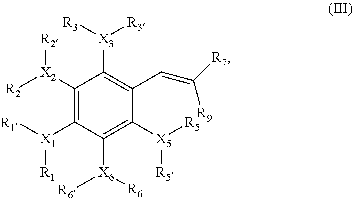
wherein:
X₁, X₂, X₃, X₅, and X₆ are each independently hydrogen, nitrogen, oxygen, sulfur, —C(O)—, or phosphorus provided that if X₁ is hydrogen, R₁ and R_{1′} are absent; if X₃ is hydrogen, R₃ and R_{3′} are absent; and if X₅ is hydrogen, R₅ and R_{5′} are absent; and provided that at least one of X₁, X₂, X₃, X₅, and X₆ is not hydrogen;
R₁, R_{1′}, R₂, R_{2′}, R₃, R_{3′}, R₅, R_{5′}, R₆, and R_{6′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
—C(O)R₈, wherein:
R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups; and
R₉ is hydrogen or an electron-withdrawing group.
or a salt thereof.

7. The method according to claim 1, wherein the matrix compound is further defined as: 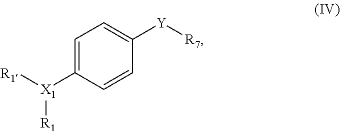
wherein:
X₁ is nitrogen, oxygen, phosphorus, —C(O)—, or sulfur;
R₁ and R_{1′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
Y is alkanediyl_{(C≤8)}, substituted alkanediyl_{(C≤8)}, alkenediyl_{(C≤8)}, substituted alkenediyl_{(C≤8)}, alkynediyl_{(C≤8)}, or substituted alkynediyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
—C(O)R₈, wherein:
R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof.

8. The method according to claim 1, wherein the matrix compound is further defined as: 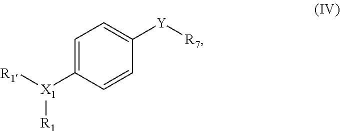
wherein:
X₁ is nitrogen, oxygen, phosphorus, —C(O)—, or sulfur;
R₁ and R_{1′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
Y is alkanediyl_{(C≤8)}, polar-substituted alkanediyl_{(C≤8)}, alkenediyl_{(C≤8)}, polar-substituted alkenediyl_{(C≤8)}, alkynediyl_{(C≤8)}, or polar-substituted alkynediyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
—C(O)R₈, wherein:
R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof.

9. The method according to a claim 1, wherein the matrix compound is further defined as: 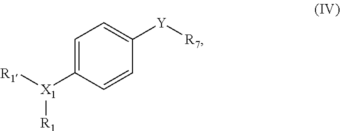
wherein:
X₁ is nitrogen or —C(O)—;
R₁ and R_{1′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
Y is alkanediyl_{(C≤8)}, substituted alkanediyl_{(C≤8)}, alkenediyl_{(C≤8)}, substituted alkenediyl_{(C≤8)}, alkynediyl_{(C≤8)}, or substituted alkynediyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
—C(O)R₈, wherein:
R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof.

10.-133. (canceled)

134. The method according to claim 1, wherein the matrix compound is further defined as: 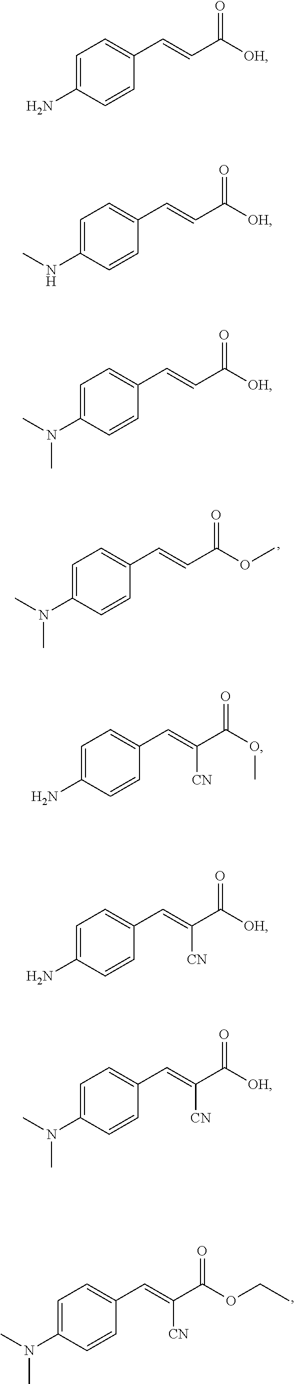 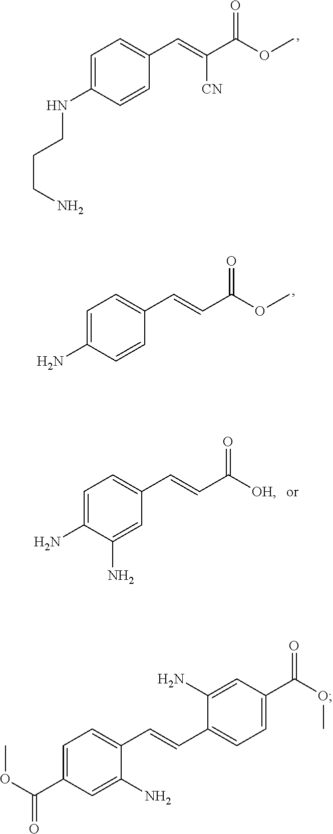 [SMILES: COC(=O)/C(C#N)=C/c1ccc(NCCCN)cc1 ; COC(=O)/C=C/c1ccc(N)cc1 ; Nc1ccc(/C=C/C(=O)O)cc1N ; COC(=O)c1ccc(/C=C/c2ccc(C(=O)OC)cc2N)c(N)c1]
or a salt thereof.

135. A matrix for use in matrix-assisted laser desorption/ionization, comprising a matrix compound having the formula: 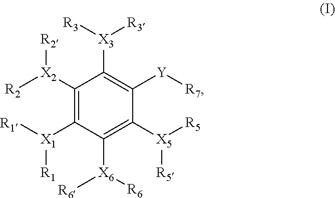
wherein:
X₁, X₂, X₃, X₅, and X₆ are each independently hydrogen, —C(O)—, or a proton-accepting atom, provided that if X₁ is hydrogen, R₁ and R_{1′} are absent; if X₃ is hydrogen, R₃ and R_{3′} are absent; and if X₅ is hydrogen, R₅ and R_{5′} are absent; and provided that at least one of X₁, X₂, X₃, X₅, and X₆ is not hydrogen;
R₁, R_{1′}, R₂, R_{2′}, R₃, R_{3′}, R₅, R_{5′}, R₆, and R_{6′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
Y is alkanediyl_{(C≤8)}, substituted alkanediyl_{(C≤8)}, alkenediyl_{(C≤8)}, substituted alkenediyl_{(C≤8)}, alkynediyl_{(C≤8)}, or substituted alkynediyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
—C(O)R₈, wherein:
R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof.

136. The matrix of claim 135, wherein the matrix compound is further defined as: 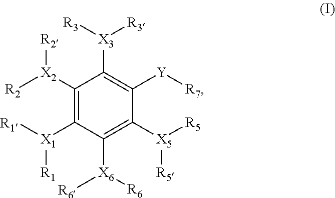
wherein:
X₁, X₂, X₃, X₅, and X₆ are each independently hydrogen, —C(O)—, or a proton-accepting atom, provided that if X₁ is hydrogen, R₁ and R_{1′} are absent; if X₃ is hydrogen, R₃ and R_{3′} are absent; and if X₅ is hydrogen, R₅ and R_{5′} are absent; and provided that at least one of X₁, X₂, X₃, X₅, and X₆ is not hydrogen;
R₁, R_{1′}, R₂, R_{2′}, R₃, R_{3′}, R₅, R_{5′}, R₆, and R_{6′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
Y is alkanediyl_{(C≤8)}, polar-substituted alkanediyl_{(C≤8)}, alkenediyl_{(C≤8)}, polar-substituted alkenediyl_{(C≤8)}, alkynediyl_{(C≤8)}, or polar-substituted alkynediyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
—C(O)R₈, wherein:
R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof;

137. The matrix of claim 135, wherein the matrix compound is further defined as: 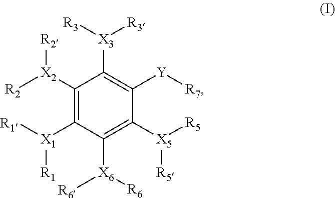
wherein:
X₁, X₂, X₃, X₅, and X₆ are each independently hydrogen, nitrogen, oxygen, sulfur, —C(O)—, or phosphorus provided that if X₁ is hydrogen, R₁ and R_{1′} are absent; if X₃ is hydrogen, R₃ and R_{3′} are absent; and if X₅ is hydrogen, R₅ and R_{5′} are absent; and provided that at least one of X₁, X₂, X₃, X₅, and X₆ is not hydrogen;
R₁, R_{1′}, R₂, R_{2′}, R₃, R_{3′}, R₅, R_{5′}, R₆, and R_{6′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
Y is alkanediyl_{(C≤8)}, polar-substituted alkanediyl_{(C≤8)}, alkenediyl_{(C≤8)}, polar-substituted alkenediyl_{(C≤8)}, alkynediyl_{(C≤8)}, or polar-substituted alkynediyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
—C(O)R₈, wherein:
R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof.

138. The matrix of claim 135, wherein the matrix compound is further defined as: 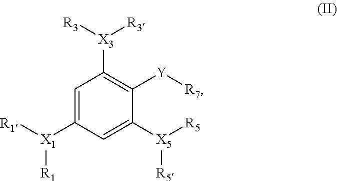
wherein:
X₁, X₃, and X₅ are each independently hydrogen, —C(O)—, or a proton-accepting atom, provided that if X₁ is hydrogen, R₁ and R_{1′} are absent; if X₃ is hydrogen, R₃ and R_{3′} are absent; and if X₅ is hydrogen, R₅ and R_{5′} are absent; and provided that at least one of X₁, X₃, and X₅ is not hydrogen;
R₁, R_{1′}, R₃, R_{3′}, R₅, and R_{5′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
Y is alkanediyl_{(C≤8)}, substituted alkanediyl_{(C≤8)}, alkenediyl_{(C≤8)}, substituted alkenediyl_{(C≤8)}, alkynediyl_{(C≤8)}, or substituted alkynediyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
—C(O)R₈, wherein:
R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof.

139. The matrix of claim 135, wherein the matrix compound is further defined as: 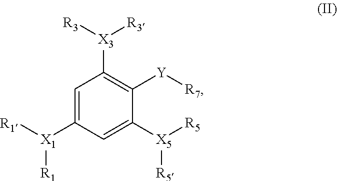
wherein:
X₁, X₃, and X₅ are each independently hydrogen, nitrogen, oxygen, sulfur, —C(O)—, or phosphorus provided that if X₁ is hydrogen, R₁ and R_{1′} are absent; if X₃ is hydrogen, R₃ and R_{3′} are absent; and if X₅ is hydrogen, R₅ and R_{5′} are absent; and provided that at least one of X₁, X₃, and X₅ is not hydrogen;
R₁, R_{1′}, R₃, R_{3′}, R₅, and R_{5′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
Y is alkanediyl_{(C≤8)}, polar-substituted alkanediyl_{(C≤8)}, alkenediyl_{(C≤8)}, polar-substituted alkenediyl_{(C≤8)}, alkynediyl_{(C≤8)}, or polar-substituted alkynediyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
—C(O)R₈, wherein:
R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof.

140. The matrix of claim 135, wherein the matrix compound is further defined as: 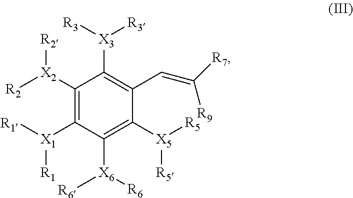
wherein:
X₁, X₂, X₃, X₅, and X₆ are each independently hydrogen, nitrogen, oxygen, sulfur, —C(O)—, or phosphorus provided that if X₁ is hydrogen, R₁ and R_{1′} are absent; if X₃ is hydrogen, R₃ and R_{3′} are absent; and if X₅ is hydrogen, R₅ and R_{5′} are absent; and provided that at least one of X₁, X₂, X₃, X₅, and X₆ is not hydrogen;
R₁, R_{1′}, R₂, R_{2′}, R₃, R_{3′}, R₅, R_{5′}, R₆, and R_{6′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
—C(O)R₈, wherein:
R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups; and
R₉ is hydrogen or an electron-withdrawing group.
or a salt thereof.

141. The matrix of claim 135, wherein the matrix compound is further defined as: 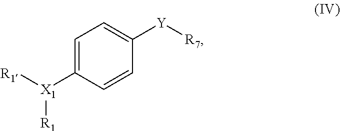
wherein:
X₁ is nitrogen, oxygen, phosphorus, —C(O)—, or sulfur;
R₁ and R_{1′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
Y is alkanediyl_{(C≤8)}, substituted alkanediyl_{(C≤8)}, alkenediyl_{(C≤8)}, psubstituted alkenediyl_{(C≤8)}, alkynediyl_{(C≤8)}, or substituted alkynediyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
—C(O)R₈, wherein:
R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof.

142. The matrix according to claim 135, wherein the matrix compound is further defined as: 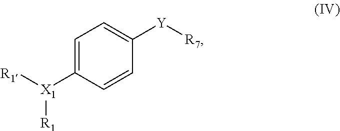
wherein:
X₁ is nitrogen, oxygen, phosphorus, —C(O)—, or sulfur;
R₁ and R_{1′} are each independently absent, hydrogen, alkyl_{(C≤8)}, substituted alkyl_{(C≤8)}, alkoxy_{(C≤8)}, substituted alkoxy_{(C≤8)}, cycloalkyl_{(C≤8)}, substituted cycloalkyl_{(C≤8)}, alkenyl_{(C≤8)}, substituted alkenyl_{(C≤8)}, alkynyl_{(C≤8)}, or substituted alkynyl_{(C≤8)};
Y is alkanediyl_{(C≤8)}, polar-substituted alkanediyl_{(C≤8)}, alkenediyl_{(C≤8)}, polar-substituted alkenediyl_{(C≤8)}, alkynediyl_{(C≤8)}, or polar-substituted alkynediyl_{(C≤8)};
R₇ is cyano, alkylsulfonyl_{(C≤8)}, substituted alkylsulfonyl_{(C≤8)}, substituted cycloalkylsulfonyl_{(C≤8)}, cycloalkylsulfonyl_{(C≤8)}, aryl_{(C≤12)}, substituted aryl_{(C≤12)}; or
—C(O)R₈, wherein:
R₈ is hydrogen, hydroxy, alkyl_{(C≤8)}, substituted alkyl_{(C≤8)}, alkenyl_{(C≤8)}, substituted alkenyl_{(C≤8)}, alkynyl_{(C≤8)}, substituted alkynyl_{(C≤8)}, cycloalkyl_{(C≤8)}, substituted cycloalkyl_{(C≤8)}; or
alkoxy_{(C≤8)}, alkenyloxy_{(C≤8)}, alkynyloxy_{(C≤8)}, cycloalkoxy_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
or a salt thereof.

143.-235. (canceled)

236. The matrix according to claim 135, wherein the matrix compound is further defined as: 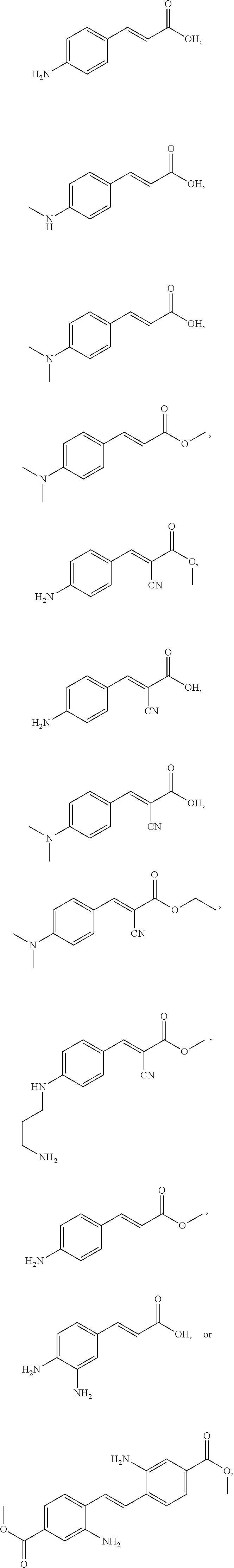
or a salt thereof.

237. A compound of the formula: 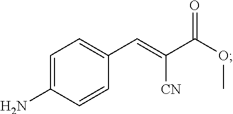
or a salt thereof.
